(19) United States
(12) Patent Application Publication
(10) Pub. No.: US 2012/0116257 A1
(43) Pub. Date: May 10, 2012

(54) Postural information system and method including determining response to subject advisory information

(75) Inventors: Eric C. Leuthardt, St. Louis, MO (US); Royce A. Levien, Lexington, MA (US)

(73) Assignee: Searete LLC

(21) Appl. No.: 13/199,730

(22) Filed: Sep. 6, 2011

Related U.S. Application Data
(63) Continuation-in-part of application No. 12/381,144, filed on Mar. 5, 2009, now abandoned.
Application No. 12/381,144 is a continuation-in-part of application No. 12/381,200, filed on Mar. 6, 2009.
Application No. 12/381,200 is a continuation-in-part of application No. 12/381,370, filed on Mar. 10, 2009.
Application No. 12/381,370 is a continuation-in-part of application No. 12/381,522, filed on Mar. 11, 2009.
Application No. 12/381,522 is a continuation-in-part of application No. 12/381,681, filed on Mar. 13, 2009.
Application No. 12/381,681 is a continuation-in-part of application No. 12/383,261, filed on Mar. 20, 2009.
Application No. 12/383,261 is a continuation-in-part of application No. 12/383,452, filed on Mar. 23, 2009.
Application No. 12/383,452 is a continuation-in-part of application No. 12/383,583, filed on Mar. 24, 2009.
Application No. 12/383,583 is a continuation-in-part of application No. 12/383,818, filed on Mar. 25, 2009.
Application No. 12/383,818 is a continuation-in-part of application No. 12/383,852, filed on Mar. 26, 2009.
Application No. 12/383,852 is a continuation-in-part of application No. 12/384,108, filed on Mar. 30, 2009.
Application No. 12/384,108 is a continuation-in-part of application No. 12/587,019, filed on Sep. 29, 2009.
Application No. 12/587,019 is a continuation-in-part of application No. 12/587,113, filed on Sep. 30, 2009.
Application No. 12/587,113 is a continuation-in-part of application No. 12/587,412, filed on Oct. 5, 2009.
Application No. 12/587,412 is a continuation-in-part of application No. 12/587,563, filed on Oct. 7, 2009.
Application No. 12/587,563 is a continuation-in-part of application No. 12/587,900, filed on Oct. 13, 2009.
Application No. 12/587,900 is a continuation-in-part of application No. 12/384,204, filed on Mar. 31, 2009.
Application No. 12/384,204 is a continuation-in-part of application No. 12/589,798, filed on Oct. 27, 2009.

(51) Int. Cl. A61B 5/11

(52) U.S. Cl. 600/595

(57) ABSTRACT

A method includes, but is not limited to: determining subject advisory information regarding one or more subjects based at least in part upon postural influencer status information including information involving one or more spatial aspects for each of two or more postural influencers of the one or more subjects, and determining a response to the subject advisory information including one or more changes regarding one or more spatial aspects for one or more of the postural influencers. In addition to the foregoing, other related method/system aspects are described in the claims, drawings, and text forming a part of the present disclosure.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC §119(e) for provisional patent applications, for any and all parent, grandparent, great-grandparent, etc. applications of the Related Application(s)). All subject matter of the Related Applications and of any and all parent, grandparent, great-grandparent, etc. applications of the Related Applications is incorporated herein by reference to the extent such subject matter is not inconsistent herewith.

### RELATED APPLICATIONS

For purposes of the USPTO extra-statutory requirements, the present application constitutes a continuation-in-part of U.S. patent application Ser. No. 12/381,144, entitled POSTURAL INFORMATION SYSTEM AND METHOD, naming Eric C. Leuthardt and Royce A. Levien, as inventors, filed 5, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/381,200, entitled POSTURAL INFORMATION SYSTEM AND METHOD, naming Eric C. Leuthardt and Royce A. Levien, as inventors, filed 6, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/381,370, entitled POSTURAL INFORMATION SYSTEM AND METHOD, naming Eric C. Leuthardt and Royce A. Levien, as inventors, filed 10, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/381,522, entitled POSTURAL INFORMATION SYSTEM AND METHOD, naming Eric C. Leuthardt and Royce A. Levien, as inventors, filed 11, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/381,681, entitled POSTURAL INFORMATION SYSTEM AND METHOD, naming Eric C. Leuthardt and Royce A. Levien, as inventors, filed 13, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/383,261, entitled POSTURAL INFORMATION SYSTEM AND METHOD, naming Eric C. Leuthardt and Royce A. Levien as inventors, filed 20, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/383,452, entitled POSTURAL INFORMATION SYSTEM AND METHOD, naming Eric C. Leuthardt and Royce A. Levien, as inventors, filed 23, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/383,583, entitled POSTURAL INFORMATION SYSTEM AND METHOD, naming Eric C. Leuthardt and Royce A. Levien, as inventors, filed 24, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/383,818, entitled POSTURAL INFORMATION SYSTEM AND METHOD, naming Eric C. Leuthardt and Royce A. Levien as inventors, filed 25, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/383,852, entitled POSTURAL INFORMATION SYSTEM AND METHOD, naming Eric C. Leuthardt and Royce A. Levien as inventors, filed 26, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/384,108, entitled POSTURAL INFORMATION SYSTEM AND METHOD, naming Eric C. Leuthardt and Royce A. Levien as inventors, filed 30, Mar., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/587,019, entitled POSTURAL INFORMATION SYSTEM AND METHOD, naming Eric C. Leuthardt and Royce A. Levien as inventors, filed 29, Sep., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/587,113, entitled POSTURAL INFORMATION SYSTEM AND METHOD, naming Eric C. Leuthardt and Royce A. Levien as inventors, filed 30, Sep., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/587,412, entitled POSTURAL INFORMATION SYSTEM AND METHOD, naming Eric C. Leuthardt and Royce A. Levien as inventors, filed 5, Oct., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/587,563, entitled POSTURAL INFORMATION SYSTEM AND METHOD, naming Eric C. Leuthardt and Royce A. Levien as inventors, filed 7, Oct., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/587,900, entitled POSTURAL INFORMATION SYSTEM AND METHOD, naming Eric C. Leuthardt and Royce A. Levien as inventors, filed 13, Oct., 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/384,204, entitled POSTURAL INFORMATION SYSTEM AND METHOD, naming Eric C. Leuthardt and Royce A. Levien as inventors, filed 31, Mar. 2009, which is currently co-pending, or is an application of which a currently co-pending application is entitled to the benefit of the filing date.

For purposes of the USPTO extra-statutory requirements, the present application constitutes a continuation-in-part of U.S. patent application Ser. No. 12/589,798, entitled POSTURAL INFORMATION SYSTEM AND METHOD INCLUDING DETERMINING RESPONSE TO SUBJECT ADVISORY INFORMATION, naming Eric C. Leuthardt and Royce A. Levien as inventors, filed 27, Oct. 2009, which is currently co-pending, or is an application of which a currently co-pending application is entitled to the benefit of the filing date.

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

### SUMMARY

A method includes, but is not limited to: determining subject advisory information regarding one or more subjects based at least in part upon postural influencer status information including information involving one or more spatial aspects for each of two or more postural influencers of the one or more subjects, and determining a response to the subject advisory information including one or more changes regarding one or more spatial aspects for one or more of the postural influencers. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

A system includes, but is not limited to: circuitry determining subject advisory information regarding one or more subjects based at least in part upon postural influencer status information including information involving one or more spatial aspects for each of two or more postural influencers of the one or more subjects, and circuitry for determining a response to the subject advisory information including one or more changes regarding one or more spatial aspects for one or more of the postural influencers. In addition to the foregoing, other method aspects are described in the claims, drawings, and text forming a part of the present disclosure.

A system includes, but is not limited to: means determining subject advisory information regarding one or more subjects based at least in part upon postural influencer status information including information involving one or more spatial aspects for each of two or more postural influencers of the one or more subjects, and means for determining a response to the subject advisory information including one or more changes regarding one or more spatial aspects for one or more of the postural influencers. In addition to the foregoing, other method aspects are described in the claims, drawings, and text forming a part of the present disclosure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 is a block diagram of a general exemplary implementation of a postural information system.

FIG. 2 is a schematic diagram depicting an exemplary environment suitable for application of a first exemplary implementation of the general exemplary implementation of the postural information system of FIG. 1.

FIG. 3 is a block diagram of an exemplary implementation of an advisory system forming a portion of an implementation of the general exemplary implementation of the postural information system of FIG. 1.

FIG. 4 is a block diagram of an exemplary implementation of modules for an advisory resource unit 102 of the advisory system 118 of FIG. 3.

FIG. 5 is a block diagram of an exemplary implementation of modules for an advisory output 104 of the advisory system 118 of FIG. 3.

FIG. 6 is a block diagram of an exemplary implementation of a status determination system (SPS) forming a portion of an implementation of the general exemplary implementation of the postural information system of FIG. 1.

FIG. 7 is a block diagram of an exemplary implementation of modules for a status determination unit 106 of the status determination system 158 of FIG. 6.

FIG. 8 is a block diagram of an exemplary implementation of modules for a status determination unit 106 of the status determination system 158 of FIG. 6.

FIG. 9 is a block diagram of an exemplary implementation of modules for a status determination unit 106 of the status determination system 158 of FIG. 6.

FIG. 10 is a block diagram of an exemplary implementation of an object forming a portion of an implementation of the general exemplary implementation of the postural information system of FIG. 1.

FIG. 11 is a block diagram of a second exemplary implementation of the general exemplary implementation of the postural information system of FIG. 1.

FIG. 12 is a block diagram of a third exemplary implementation of the general exemplary implementation of the postural information system of FIG. 1.

FIG. 13 is a block diagram of a fourth exemplary implementation of the general exemplary implementation of the postural information system of FIG. 1.

FIG. 14 is a block diagram of a fifth exemplary implementation of the general exemplary implementation of the postural information system of FIG. 1.

FIG. 15 is a high-level flowchart illustrating an operational flow O10 representing exemplary operations related to determining subject advisory information regarding one or more subjects based at least in part upon postural influencer status information including information involving one or more spatial aspects for each of two or more postural influencers of the one or more subjects, and determining a response to the subject advisory information including one or more changes regarding one or more spatial aspects for one or more of the postural influencers at least associated with the depicted exemplary implementations of the postural information system.

FIG. 16 is a high-level flowchart including exemplary implementations of operation O11 of FIG. 15.

FIG. 17 is a high-level flowchart including exemplary implementations of operation O11 of FIG. 15.

FIG. 18 is a high-level flowchart including exemplary implementations of operation O11 of FIG. 15.

FIG. 19 is a high-level flowchart including exemplary implementations of operation O12 of FIG. 15.

FIG. 20 is a high-level flowchart including exemplary implementations of operation O12 of FIG. 15.

FIG. 21 is a high-level flowchart including exemplary implementations of operation O12 of FIG. 15.

FIG. 22 is a high-level flowchart including exemplary implementations of operation O12 of FIG. 15.

FIG. 23 is a high-level flowchart including exemplary implementations of operation O12 of FIG. 15.

FIG. 24 is a high-level flowchart illustrating an operational flow O20 representing exemplary operations related to obtaining postural influencer status information including information regarding one or more spatial aspects of one or more first postural influencers of one or more subjects with respect to a second postural influencer of the one or more subjects, determining subject advisory information regarding one or more subjects based at least in part upon postural influencer status information including information involving one or more spatial aspects for each of two or more postural influencers of the one or more subjects, and determining a response to the subject advisory information including one or more changes regarding one or more spatial aspects for one or more of the postural influencers at least associated with the depicted exemplary implementations of the postural information system.

FIG. 25 is a high-level flowchart including exemplary implementations of operation O21 of FIG. 24.

FIG. 26 is a high-level flowchart including exemplary implementations of operation O21 of FIG. 24.

FIG. 27 is a high-level flowchart including exemplary implementations of operation O21 of FIG. 24.

FIG. 28 is a high-level flowchart including exemplary implementations of operation O21 of FIG. 24.

FIG. 29 is a high-level flowchart including exemplary implementations of operation O21 of FIG. 24.

FIG. 30 is a high-level flowchart including exemplary implementations of operation O21 of FIG. 24.

FIG. 31 is a high-level flowchart including exemplary implementations of operation O21 of FIG. 24.

FIG. 32 is a high-level flowchart including exemplary implementations of operation O21 of FIG. 24.

FIG. 33 is a high-level flowchart including exemplary implementations of operation O21 of FIG. 24.

FIG. 34 is a high-level flowchart including exemplary implementations of operation O21 of FIG. 24.

FIG. 35 is a high-level flowchart illustrating an operational flow O30 representing exemplary operations related to obtaining postural influencer status information including information regarding one or more spatial aspects of one or more first postural influencers of one or more subjects with respect to a second postural influencer of the one or more subjects, obtaining subject status information associated with one or more postural aspects regarding one or more subjects of one or more of the first postural influencers, determining subject advisory information regarding one or more subjects based at least in part upon postural influencer status information including information involving one or more spatial aspects for each of two or more postural influencers of the one or more subjects, and determining a response to the subject advisory information including one or more changes regarding one or more spatial aspects for one or more of the postural influencers at least associated with the depicted exemplary implementations of the postural information system.

FIG. 36 is a high-level flowchart including exemplary implementations of operation O32 of FIG. 35.

FIG. 37 is a high-level flowchart including exemplary implementations of operation O32 of FIG. 35.

FIG. 38 is a high-level flowchart including exemplary implementations of operation O32 of FIG. 35.

FIG. 39 is a high-level flowchart including exemplary implementations of operation O32 of FIG. 35.

FIG. 40 is a high-level flowchart including exemplary implementations of operation O32 of FIG. 35.

FIG. 41 is a high-level flowchart including exemplary implementations of operation O32 of FIG. 35.

FIG. 42 is a high-level flowchart including exemplary implementations of operation O32 of FIG. 35.

FIG. 43 is a high-level flowchart including exemplary implementations of operation O32 of FIG. 35.

FIG. 44 is a high-level flowchart including exemplary implementations of operation O32 of FIG. 35.

FIG. 45 illustrates a partial view of a system S100 that includes a computer program for executing a computer process on a computing device.

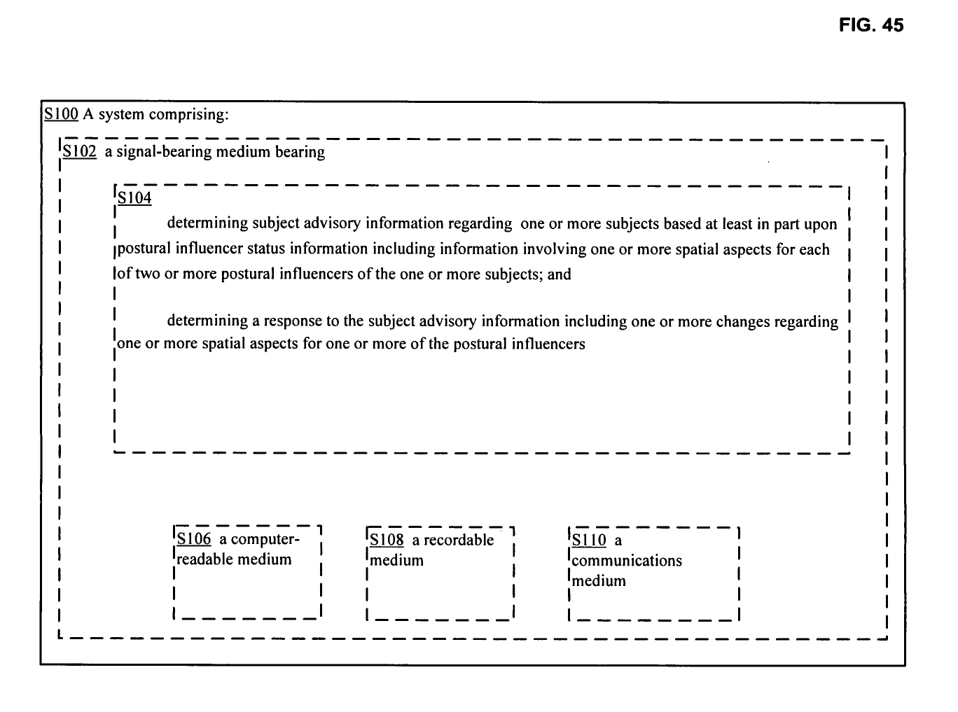

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

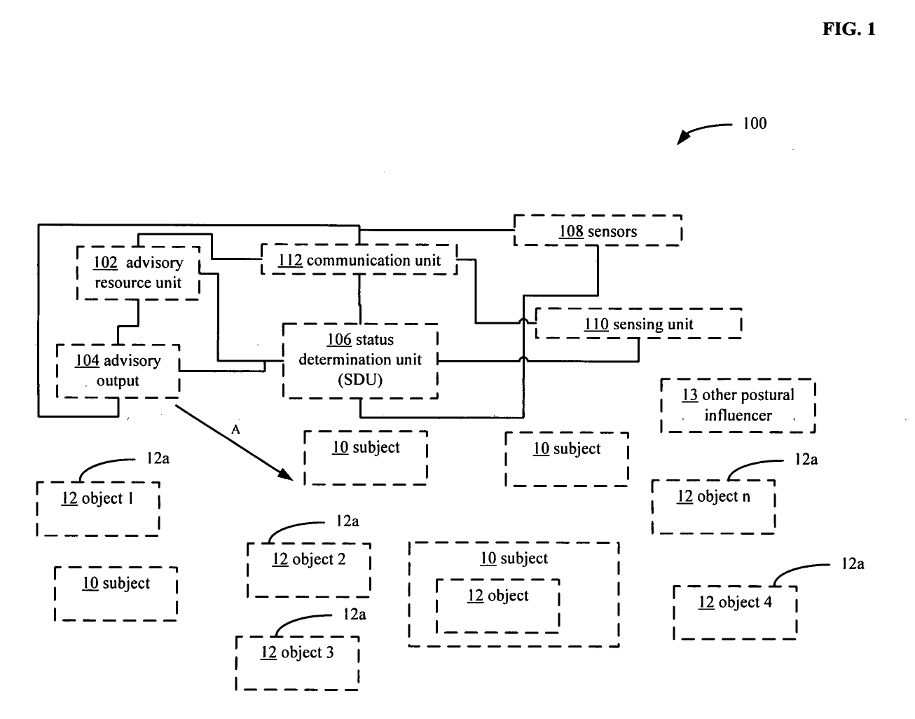

An exemplary environment is depicted in FIG. 1 in which one or more aspects of various embodiments may be implemented. In the illustrated environment, a general exemplary implementation of a system 100 may include at least an advisory resource unit 102 that is configured to determine advisory information associated at least in part with spatial aspects, such as posture, of at least portions of one or more subjects 10. In the following, one of the subjects 10 depicted in FIG. 1 will be discussed for convenience since in many of the implementations only one subject would be present, but is not intended to limit use of the system 100 to only one concurrent subject.

The subject 10 is depicted in FIG. 1 in an exemplary spatial association with a plurality of objects 12 and/or with one or more surfaces 12a thereof. Other postural influencers 13 are also included besides the objects 12 and the subjects 10. Such spatial association can influence spatial aspects of the subject 10 such as posture of the subject and thus can be used by the system 100 to determine advisory information regarding spatial aspects, such as posture, of the subject. As depicted by one of the objects 12 overlaid on to one of the subjects 10, one or more of the objects can be assigned to monitor postural status of one or more of the subjects regarding such aspects as position, location, orientation, and/or conformation of one or more portions of the subject.

For example, the subject 10 can be a human, animal, robot, or other that can have a posture that can be adjusted such that given certain objectives, conditions, environments and other factors, a certain posture or range or other plurality of postures for the subject 10 may be more desirable than one or more other postures. In implementations, desirable posture for the subject 10 may vary over time given changes in one or more associated factors.

One of the subjects 10, one of the objects 12, and/or one of the postural influencers 13 can be a postural influencer by somehow influencing the posture of one or more of the subjects 10. Postural influence can include, but is not limited to, touch (wherein a subject being influenced has a posture to accommodate physically touching or detecting pressure, vibration, or other touch oriented sensations associated with the postural influencer), visual (wherein a subject being influenced has a posture to accommodate seeing or otherwise detecting light associated with the postural influencer), audio (wherein a subject being influenced has a posture to accommodate hearing or otherwise detecting sound from the postural influencer), and/or scent (wherein a subject being influenced has a posture to accommodate smelling or otherwise detecting scent from the postural influencer). Furthermore in some implementations, some postural influencers can exchange postural influence with one another or have other sorts of combinational postural influence with subsets of each other.

For instance, in some implementations some of the objects 12 can include multiple display screens with some of the screens having large areas with more than one display element to display different types of presentations simultaneously. This can involve one or more of the subjects 10 as observers of the display screens to change posture to view the more than one display screens and more than one display elements within one or more of the larger display screens.

Implementations can be found in conference rooms, auditoriums, and/or other meeting places and/or where kiosks and/or other sorts of publicly shared displays exist where a plurality of the subjects 10 can be present. In some implementations, some of the subjects 10 can be presenters to other subjects and can also be observers of the display screens. Accordingly, some of the subjects can be postural influencers of other subjects as well as having their posture influenced by other postural influencers. For instance, in a conference room there may be many display screens, some having multiple elements. There can be one or more discussions occurring with one or more presenters involved. Postural status of the various subjects 10 as observers, presenters or both can be influenced by placement, orientation and other factors involved with the display screens, the presenters, and the observers.

Various approaches have introduced ways to determine physical status of a living subject with sensors being directly attached to the subject. Sensors can be used to distinguishing lying, sitting, and standing positions. This sensor data can then be stored in a storage device as a function of time. Multiple points or multiple intervals of the time dependent data can be used to direct a feedback mechanism to provide information or instruction in response to the time dependent output indicating too little activity, too much time with a joint not being moved beyond a specified range of motion, too many motions beyond a specified range of motion, or repetitive activity that can cause repetitive stress injury, etc.

Approaches have included a method for preventing computer induced repetitive stress injuries (CRSI) that records operation statistics of the computer, calculates a computer subject's weighted fatigue level; and will automatically remind a subject of necessary responses when the fatigue level reaches a predetermined threshold. Some have measured force, primarily due to fatigue, such as with a finger fatigue measuring system, which measures the force output from fingers while the fingers are repetitively generating forces as they strike a keyboard. Force profiles of the fingers have been generated from the measurements and evaluated for fatigue. Systems have been used clinically to evaluate patients, to ascertain the effectiveness of clinical intervention, pre-employment screening, to assist in minimizing the incidence of repetitive stress injuries at the keyboard, mouse, joystick, and to monitor effectiveness of various finger strengthening systems. Systems have also been used in a variety of different applications adapted for measuring forces produced during performance of repetitive motions.

Others have introduced support surfaces and moving mechanisms for automatically varying orientation of the support surfaces in a predetermined manner over time to reduce or eliminate the likelihood of repetitive stress injury as a result of performing repetitive tasks on or otherwise using the support surface. By varying the orientation of the support surface, e.g., by moving and/or rotating the support surface over time, repetitive tasks performed on the support surface are modified at least subtly to reduce the repetitiveness of the individual motions performed by an operator.

Some have introduced attempts to reduce, prevent, or lessen the incidence and severity of repetitive strain injuries (“RSI”) with a combination of computer software and hardware that provides a “prompt” and system whereby the computer operator exercises their upper extremities during data entry and word processing thereby maximizing the excursion (range of motion) of the joints involved directly and indirectly in computer operation. Approaches have included 1) specialized target means with optional counters which serves as “goals” or marks towards which the hands of the typist are directed during prolonged key entry, 2) software that directs the movement of the limbs to and from the keyboard, and 3) software that individualizes the frequency and intensity of the exercise sequence.

Others have included a wrist-resting device having one or both of a heater and a vibrator in the device wherein a control system is provided for monitoring subject activity and weighting each instance of activity according to stored parameters to accumulate data on subject stress level. In the event a prestored stress threshold is reached, a media player is invoked to provide rest and exercise for the subject.

Others have introduced biometrics authentication devices to identify characteristics of a body from captured images of the body and to perform individual authentication. The device guides a subject, at the time of verification, to the image capture state at the time of registration of biometrics characteristic data. At the time of registration of biometrics characteristic data, body image capture state data is extracted from an image captured by an image capture unit and is registered in a storage unit, and at the time of verification the registered image capture state data is read from the storage unit and is compared with image capture state data extracted at the time of verification, and guidance of the body is provided. Alternatively, an outline of the body at the time of registration, taken from image capture state data at the time of registration, is displayed.

Others have introduced mechanical models of human bodies having rigid segments connected with joints. Such models include articulated rigid-multibody models used as a tool for investigation of the injury mechanism during car crush events. Approaches can be semi-analytical and can be based on symbolic derivatives of the differential equations of motion. They can illustrate the intrinsic effect of human body geometry and other influential parameters on head acceleration.

Some have introduced methods of effecting an analysis of behaviors of substantially all of a plurality of real segments together constituting a whole human body, by conducting a simulation of the behaviors using a computer under a predetermined simulation analysis condition, on the basis of a numerical whole human body model provided by modeling on the computer the whole human body in relation to a skeleton structure thereof including a plurality of bones, and in relation to a joining structure of the whole human body which joins at least two real segments of the whole human body and which is constructed to have at least one real segment of the whole human body, the at least one real segment being selected from at least one ligament, at least one tendon, and at least one muscle, of the whole human body.

Others have introduced spatial body position detection to calculate information on a relative distance or positional relationship between an interface section and an item by detecting an electromagnetic wave transmitted through the interface section, and using the electromagnetic wave from the item to detect a relative position of the item with respective to the interface section. Information on the relative spatial position of an item with respect to an interface section that has an arbitrary shape and deals with transmission of information or signal from one side to the other side of the interface section is detected with a spatial position detection method. An electromagnetic wave radiated from the item and transmitted through the interface section is detected by an electromagnetic wave detection section, and based on the detection result; information on spatial position coordinates of the item is calculated by a position calculation section.

Some introduced a template-based approach to detecting human silhouettes in a specific walking pose with templates having short sequences of 2D silhouettes obtained from motion capture data. Motion information is incorporated into the templates to help distinguish actual people who move in a predictable way from static objects whose outlines roughly resemble those of humans. During the training phase we use statistical learning techniques to estimate and store the relevance of the different silhouette parts to the recognition task. At run-time, Chamfer distance is converted to meaningful probability estimates. Particular templates handle six different camera views, excluding the frontal and back view, as well as different scales and are particularly useful for both indoor and outdoor sequences of people walking in front of cluttered backgrounds and acquired with a moving camera, which makes techniques such as background subtraction impractical.

Further discussion of approaches introduced by others can be found in U.S. Pat. Nos. 5,792,025, 5,868,647, 6,161,806, 6,352,516, 6,673,026, 6,834,436, 7,210,240, 7,248,995, 7,248,995, and 7,353,151; U.S. Patent Application Nos. 20040249872, and 20080226136; “Sensitivity Analysis of the Human Body Mechanical Model”, Zeitschrift für angewandte Mathematik and Mechanik, 2000, vol. 80, pp. S343-S344, SUP2 (6 ref.); and “Human Body Pose Detection Using Bayesian Spatio-Temporal Templates,” Computer Vision and Image Understanding, Volume 104, Issues 2-3, November-December 2006, Pages 127-139 M. Dimitrijevic, V. Lepetit and P. Fua.

Exemplary implementations of the system 100 can also include an advisory output 104, a status determination unit 106, one or more sensors 108, a sensing unit 110, and communication unit 112. In some implementations, the advisory output 104 receives messages containing advisory information from the advisory resource unit 102. In response to the received advisory information, the advisory output 104 sends an advisory to the subject 10 in a suitable form containing information such as related to spatial aspects of the subject and/or one or more of the objects 12.

A suitable form of the advisory can include visual, audio, touch, temperature, vibration, flow, light, radio frequency, other electromagnetic, and/or other aspects, media, and/or indicators that could serve as a form of input to the subject 10.

Spatial aspects can be related to posture and/or other spatial aspects and can include location, position, orientation, visual placement, visual appearance, and/or conformation of one or more portions of one or more of the subject 10 and/or one or more portions of one or more of the object 12. Location can involve information related to landmarks or other objects. Position can involve information related to a coordinate system or other aspect of cartography. Orientation can involve information related to a three dimensional axis system. Visual placement can involve such aspects as placement of display features, such as icons, scene windows, scene widgets, graphic or video content, or other visual features on a display such as a display monitor. Visual appearance can involve such aspects as appearance, such as sizing, of display features, such as icons, scene windows, scene widgets, graphic or video content, or other visual features on a display such as a display monitor. Conformation can involve how various portions including appendages are arranged with respect to one another. For instance, one of the objects 12 may be able to be folded or have moveable arms or other structures or portions that can be moved or re-oriented to result in different conformations.

Examples of such advisories can include but are not limited to aspects involving re-positioning, re-orienting, and/or re-configuring the subject 10 and/or one or more of the objects 12. For instance, the subject 10 may use some of the objects 12 through vision of the subject and other of the objects through direct contact by the subject. A first positioning of the objects 12 relative to one another may cause the subject 10 to have a first posture in order to accommodate the subject's visual or direct contact interaction with the objects. An advisory may include content to inform the subject 10 to change to a second posture by re-positioning the objects 12 to a second position so that visual and direct contact use of the objects 12 can be performed in the second posture by the subject. Advisories that involve one or more of the objects 12 as display devices may involve spatial aspects such as visual placement and/or visual appearance and can include, for example, modifying how or what content is being displayed on one or more of the display devices.

The system 100 can also include a status determination unit (SDU) 106 that can be configured to determine physical status of the objects 12 and also in some implementations determine physical status of the subject 10 as well. Physical status can include spatial aspects such as location, position, orientation, visual placement, visual appearance, and/or conformation of the objects 12 and optionally the subject 10. In some implementations, physical status can include other aspects as well.

The status determination unit 106 can furnish determined physical status that the advisory resource unit 102 can use to provide appropriate messages to the advisory output 104 to generate advisories for the subject 10 regarding posture or other spatial aspects of the subject with respect to the objects 12. In implementations, the status determination unit 106 can use information regarding the objects 12 and in some cases the subject 10 from one or more of the sensors 108 and/or the sensing unit 110 to determine physical status.

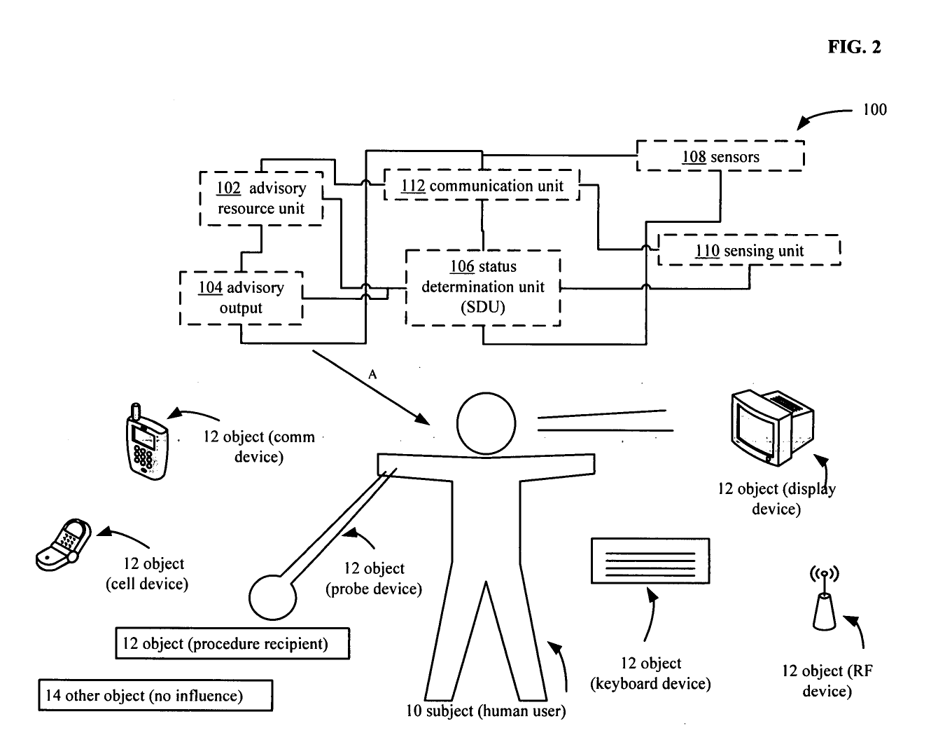

As shown in FIG. 2, an exemplary implementation of the system 100 is applied to an environment in which the objects 12 include a communication device, a cellular device, a probe device servicing a procedure recipient, a keyboard device, a display device, and an RF device and wherein the subject 10 is a human. Also shown is an other object 14 that does not influence the physical status of the subject 10, for instance, the subject is not required to view, touch, or otherwise interact with the other object as to affect the physical status of the subject due to an interaction. The environment depicted in FIG. 2 is merely exemplary and is not intended to limit what types of the subject 10, the objects 12, and the environments can be involved with the system 100. The environments that can be used with the system 100 are far ranging and can include any sort of situation in which the subject 10 is being influenced regarding posture or other spatial aspects of the subject by one or more spatial aspects of the objects 12.

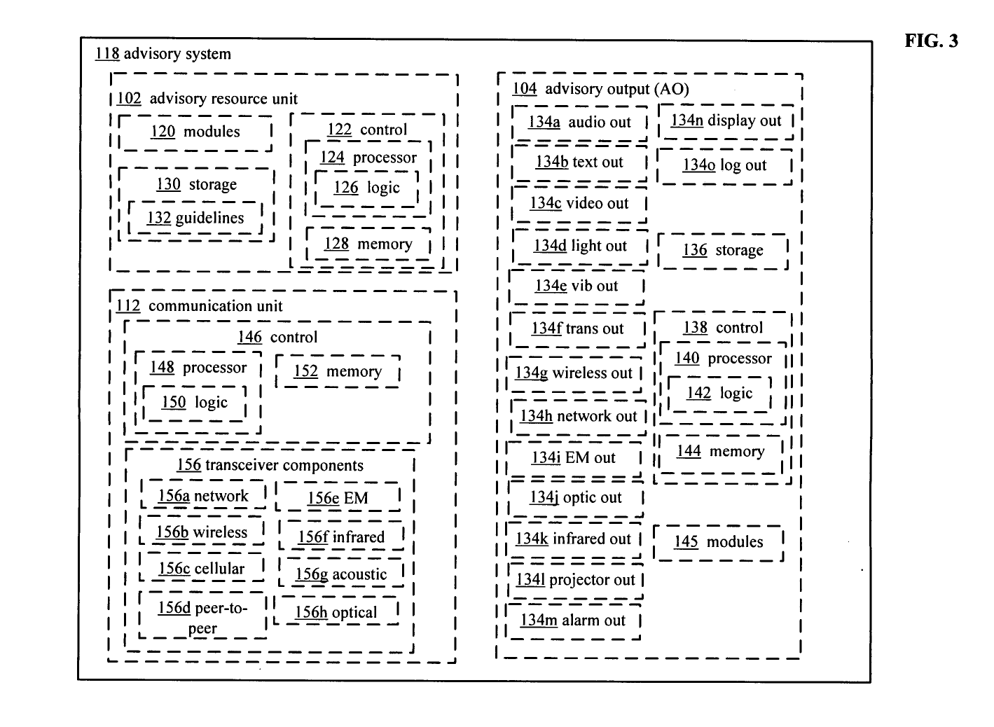

An advisory system 118 is shown in FIG. 3 to optionally include instances of the advisory resource unit 102, the advisory output 104 and a communication unit 112. The advisory resource unit 102 is depicted to have modules 120, a control unit 122 including a processor 124, a logic unit 126, and a memory unit 128, and having a storage unit 130 including guidelines 132. The advisory output 104 is depicted to include an audio output 134a, a textual output 134b, a video output 134c, a light output 134d, a vibrator output 134e, a transmitter output 134f, a wireless output 134g, a network output 134h, an electromagnetic output 134i, an optic output 134j, an infrared output 134k, a projector output 134l, an alarm output 134m, a display output 134n, and a log output 134o, a storage unit 136, a control 138, a processor 140 with a logic unit 142, a memory 144, and modules 145.

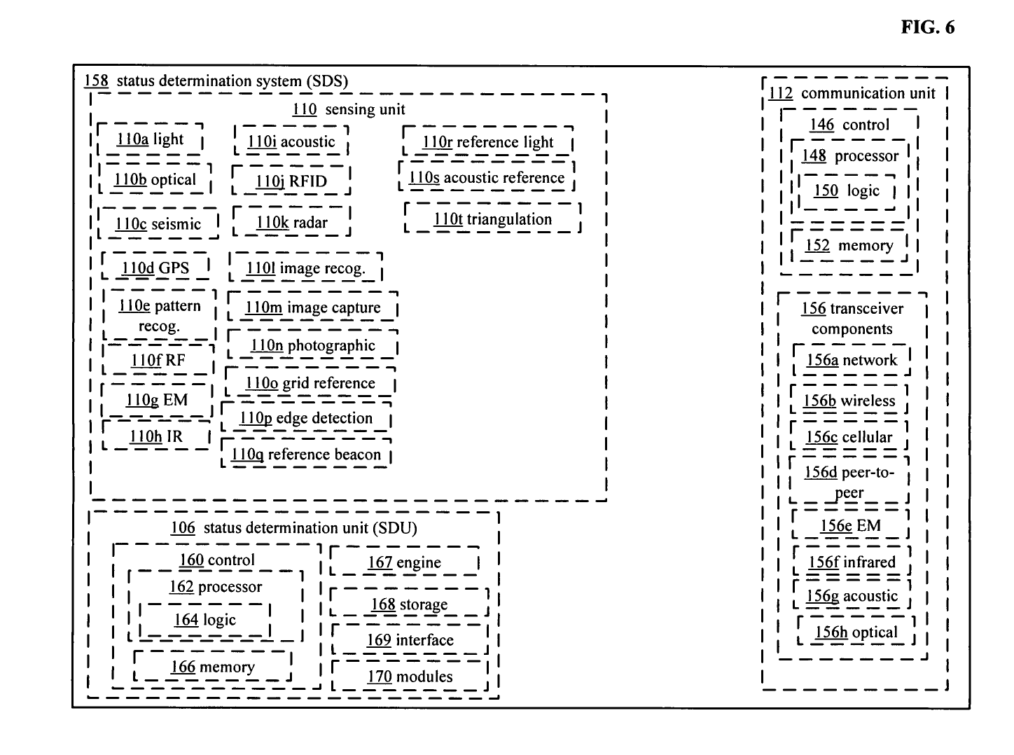

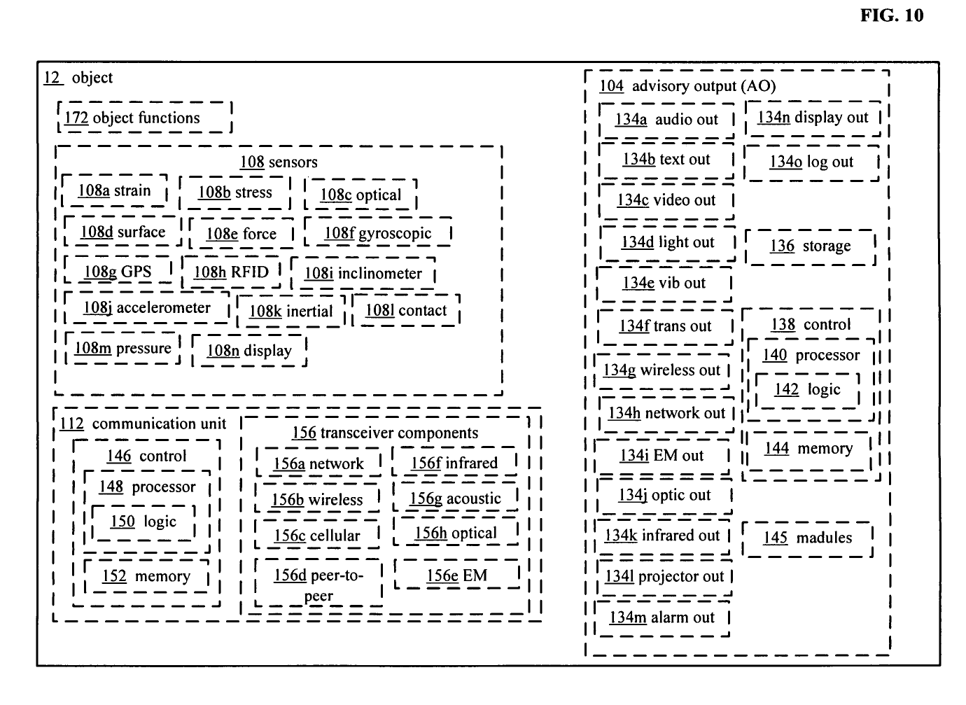

The communication unit 112 is depicted in FIG. 3 to optionally include a control unit 146 including a processor 148, a logic unit 150, and a memory 152 and to have transceiver components 156 including a network component 156a, a wireless component 156b, a cellular component 156c, a peer-to-peer component 156d, an electromagnetic (EM) component 156e, an infrared component 156f, an acoustic component 156g, and an optical component 156h. In general, similar or corresponding systems, units, components, or other parts are designated with the same reference number throughout, but each with the same reference number can be internally composed differently. For instance, the communication unit 112 is depicted in various Figures as being used by various components, systems, or other items such as in instances of the advisory system in FIG. 3, in the status determination system of FIG. 6, and in the object of FIG. 10, but is not intended that the same instance or copy of the communication unit 112 is used in all of these cases, but rather various versions of the communication unit having different internal composition can be used to satisfy the requirements of each specific instance.

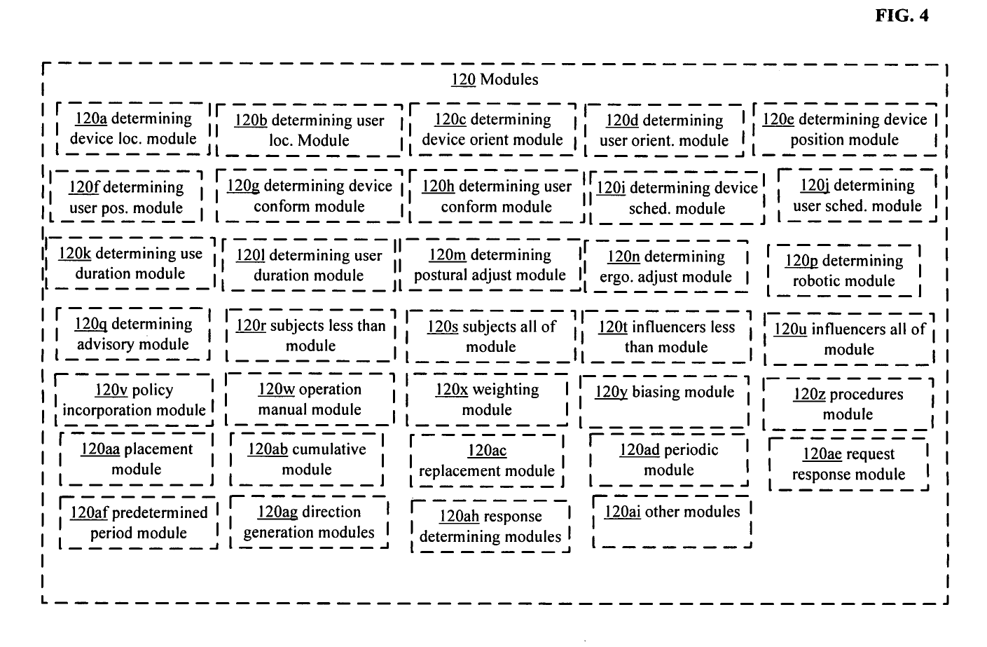

The modules 120 is further shown in FIG. 4 to optionally include a determining device location module 120a, a determining subject location module 120b, a determining device orientation module 120c, a determining subject orientation module 120d, a determining device position module 120e, a determining subject position module 120f, a determining device conformation module 120g, a determining subject conformation module 120h, a determining device schedule module 120i, a determining subject schedule module 120j, a determining use duration module 120k, a determining subject duration module 120l, a determining postural adjustment module 120m, a determining ergonomic adjustment module 120n, a determining robotic module 120p, a determining advisory module 120q, a subjects less than module 120r, a subjects all of module 120s, an influencers less than module 120t, an influencers all of module 120u, a policy incorporation module 120v, an operation manual module 120w, a weighting module 120x, a biasing module 120y, a procedures module 120z, a placement module 120aa, a cumulative module 120ab, a replacement module 120ac, a periodic module 120ad, a request response module 120ae, a predetermined period module 120af, a direction generation module 120ag, a response determining module 120ah, and an other modules 120ai.

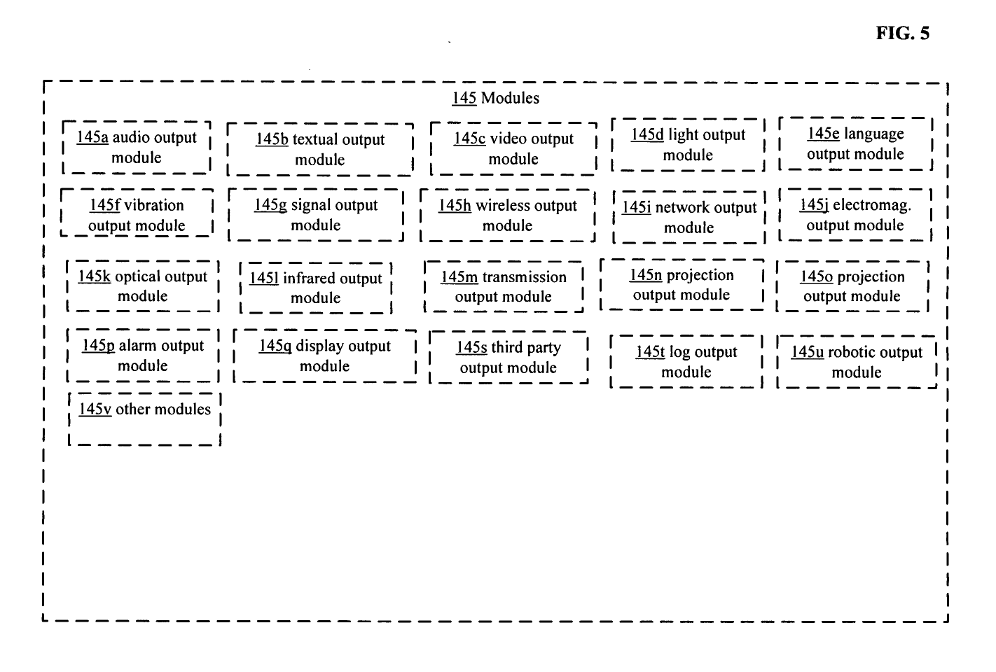

The modules 145 is further shown in FIG. 5 to optionally include an audio output module 145a, a textual output module 145b, a video output module 145c, a light output module 145d, a language output module 145e, a vibration output module 145f, a signal output module 145g, a wireless output module 145h, a network output module 145i, an electromagnetic output module 145j, an optical output module 145k, an infrared output module 145l, a transmission output module 145m, a projection output module 145n, a projection output module 145o, an alarm output module 145p, a display output module 145q, a third party output module 145s, a log output module 145t, a robotic output module 145u, and an other modules 145v.

A status determination system (SDS) 158 is shown in FIG. 6 to optionally include the communication unit 112, the sensing unit 110, and the status determination unit 106. The sensing unit 110 is further shown to optionally include a light based sensing component 110a, an optical based sensing component 110b, a seismic based sensing component 110c, a global positioning system (GPS) based sensing component 110d, a pattern recognition based sensing component 110e, a radio frequency based sensing component 110f, an electromagnetic (EM) based sensing component 110g, an infrared (IR0 sensing component 110h, an acoustic based sensing component 110i, a radio frequency identification (RFID) based sensing component 110j, a radar based sensing component 110k, an image recognition based sensing component 110l, an image capture based sensing component 110m, a photographic based sensing component 110n, a grid reference based sensing component 110o, an edge detection based sensing component 110p, a reference beacon based sensing component 110q, a reference light based sensing component 110r, an acoustic reference based sensing component 110s, and a triangulation based sensing component 110t.

The sensing unit 110 can include use of one or more of its various based sensing components to acquire information on physical status of the subject 10 and the objects 12 even when the subject and the objects maintain a passive role in the process. For instance, the light based sensing component 110a can include light receivers to collect light from emitters or ambient light that was reflected off or otherwise have interacted with the subject 10 and the objects 12 to acquire postural influencer status information regarding the subject and the objects. The optical based sensing component 110b can include optical based receivers to collect light from optical emitters that have interacted with the subject 10 and the objects 12 to acquire postural influencer status information regarding the subject and the objects.

For instance, the seismic based sensing component 110c can include seismic receivers to collect seismic waves from seismic emitters or ambient seismic waves that have interacted with the subject 10 and the objects 12 to acquire postural influencer status information regarding the subject and the objects. The global positioning system (GPS) based sensing component 110d can include GPS receivers to collect GPS information associated with the subject 10 and the objects 12 to acquire postural influencer status information regarding the subject and the objects. The pattern recognition based sensing component 110e can include pattern recognition algorithms to operate with the determination engine 167 of the status determination unit 106 to recognize patterns in information received by the sensing unit 110 to acquire postural influencer status information regarding the subject and the objects.

For instance, the radio frequency based sensing component 110f can include radio frequency receivers to collect radio frequency waves from radio frequency emitters or ambient radio frequency waves that have interacted with the subject 10 and the objects 12 to acquire postural influencer status information regarding the subject and the objects. The electromagnetic (EM) based sensing component 110g, can include electromagnetic frequency receivers to collect electromagnetic frequency waves from electromagnetic frequency emitters or ambient electromagnetic frequency waves that have interacted with the subject 10 and the objects 12 to acquire postural influencer status information regarding the subject and the objects. The infrared sensing component 110h can include infrared receivers to collect infrared frequency waves from infrared frequency emitters or ambient infrared frequency waves that have interacted with the subject 10 and the objects 12 to acquire postural influencer status information regarding the subjects and the objects.

For instance, the acoustic based sensing component 110 can include acoustic frequency receivers to collect acoustic frequency waves from acoustic frequency emitters or ambient acoustic frequency waves that have interacted with the subject 10 and the objects 12 to acquire postural influencer status information regarding the subjects and the objects. The radio frequency identification (RFID) based sensing component 110j can include radio frequency receivers to collect radio frequency identification signals from RFID emitters associated with the subject 10 and the objects 12 to acquire postural influencer status information regarding the subjects and the objects. The radar based sensing component 110k can include radar frequency receivers to collect radar frequency waves from radar frequency emitters or ambient radar frequency waves that have interacted with the subject 10 and the objects 12 to acquire postural influencer status information regarding the subjects and the objects.

The image recognition based sensing component 110l can include image receivers to collect images of the subject 10 and the objects 12 and one or more image recognition algorithms to recognition aspects of the collected images optionally in conjunction with use of the determination engine 167 of the status determination unit 106 to acquire postural influencer status information regarding the subjects and the objects.

The image capture based sensing component 110m can include image receivers to collect images of the subject 10 and the objects 12 to acquire postural influencer status information regarding the subjects and the objects. The photographic based sensing component 110n can include photographic cameras to collect photographs of the subject 10 and the objects 12 to acquire postural influencer status information regarding the subjects and the objects.

The grid reference based sensing component 110o can include a grid of sensors (such as contact sensors, photo-detectors, optical sensors, acoustic sensors, infrared sensors, or other sensors) adjacent to, in close proximity to, or otherwise located to sense one or more spatial aspects of the objects 12 such as location, position, orientation, visual placement, visual appearance, and/or conformation. The grid reference based sensing component 110o can also include processing aspects to prepare sensed information for the status determination unit 106.

The edge detection based sensing component 110p can include one or more edge detection sensors (such as contact sensors, photo-detectors, optical sensors, acoustic sensors, infrared sensors, or other sensors) adjacent to, in close proximity to, or otherwise located to sense one or more spatial aspects of the objects 12 such as location, position, orientation, visual placement, visual appearance, and/or conformation. The edge detection based sensing component 110p can also include processing aspects to prepare sensed information for the status determination unit 106.

The reference beacon based sensing component 110q can include one or more reference beacon emitters and receivers (such as acoustic, light, optical, infrared, or other) located to send and receive a reference beacon to calibrate and/or otherwise detect one or more spatial aspects of the objects 12 such as location, position, orientation, visual placement, visual appearance, and/or conformation. The reference beacon based sensing component 110q can also include processing aspects to prepare sensed information for the status determination unit 106.

The reference light based sensing component 110r can include one or more reference light emitters and receivers located to send and receive a reference light to calibrate and/or otherwise detect one or more spatial aspects of the objects 12 such as location, position, orientation, visual placement, visual appearance, and/or conformation. The reference light based sensing component 110r can also include processing aspects to prepare sensed information for the status determination unit 106.

The acoustic reference based sensing component 110s can include one or more acoustic reference emitters and receivers located to send and receive an acoustic reference signal to calibrate and/or otherwise detect one or more spatial aspects of the objects 12 such as location, position, orientation, visual placement, visual appearance, and/or conformation. The acoustic reference based sensing component 110s can also include processing aspects to prepare sensed information for the status determination unit 106.

The triangulation based sensing component 110t can include one or more emitters and receivers located to send and receive signals to calibrate and/or otherwise detect using triangulation methods one or more spatial aspects of the objects 12 such as location, position, orientation, visual placement, visual appearance, and/or conformation. The triangulation based sensing component 110t can also include processing aspects to prepare sensed information for the status determination unit 106.

The status determination unit 106 is further shown in FIG. 6 to optionally include a control unit 160, a processor 162, a logic unit 164, a memory 166, a determination engine 167, a storage unit 168, an interface 169, and modules 170.

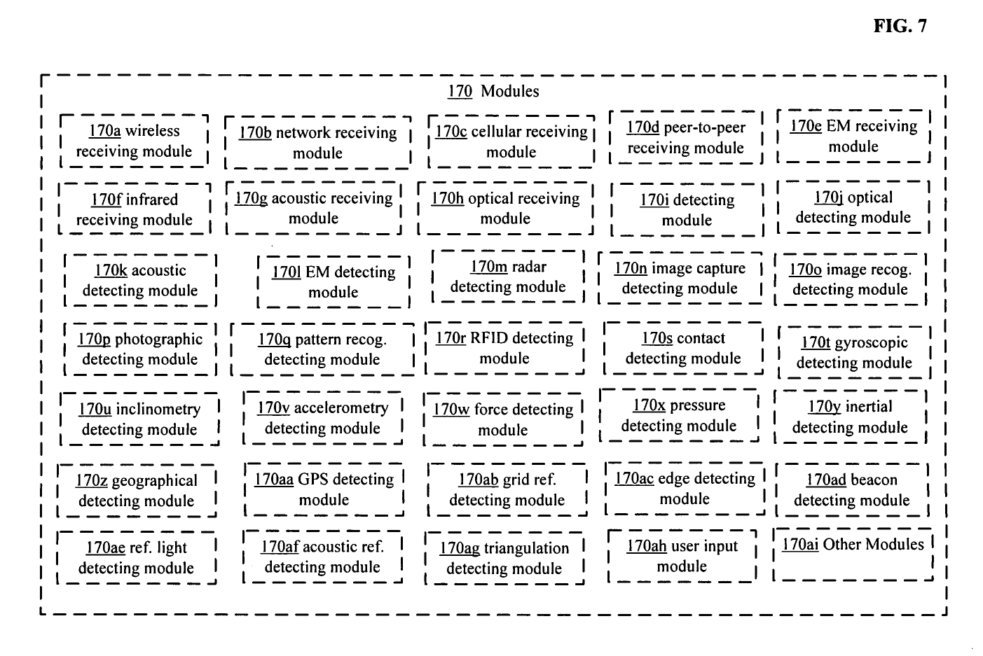

The modules 170 is further shown in FIG. 7 to optionally include a wireless receiving module 170a, a network receiving module 170b, cellular receiving module 170c, a peer-to-peer receiving module 170d, an electromagnetic receiving module 170e, an infrared receiving module 170f, an acoustic receiving module 170g, an optical receiving module 170h, a detecting module 170i, an optical detecting module 170j, an acoustic detecting module 170k, an electromagnetic detecting module 170l, a radar detecting module 170m, an image capture detecting module 170n, an image recognition detecting module 170o, a photographic detecting module 170p, a pattern recognition detecting module 170q, a radiofrequency detecting module 170r, a contact detecting module 170s, a gyroscopic detecting module 170t, an inclinometry detecting module 170u, an accelerometry detecting module 170v, a force detecting module 170w, a pressure detecting module 170x, an inertial detecting module 170y, a geographical detecting module 170z, a global positioning system (GPS) detecting module 170aa, a grid reference detecting module 170ab, an edge detecting module 170ac, a beacon detecting module 170ad, a reference light detecting module 170ae, an acoustic reference detecting module 170af, a triangulation detecting module 170ag, a subject input module 170ah, and an other modules 170ai.

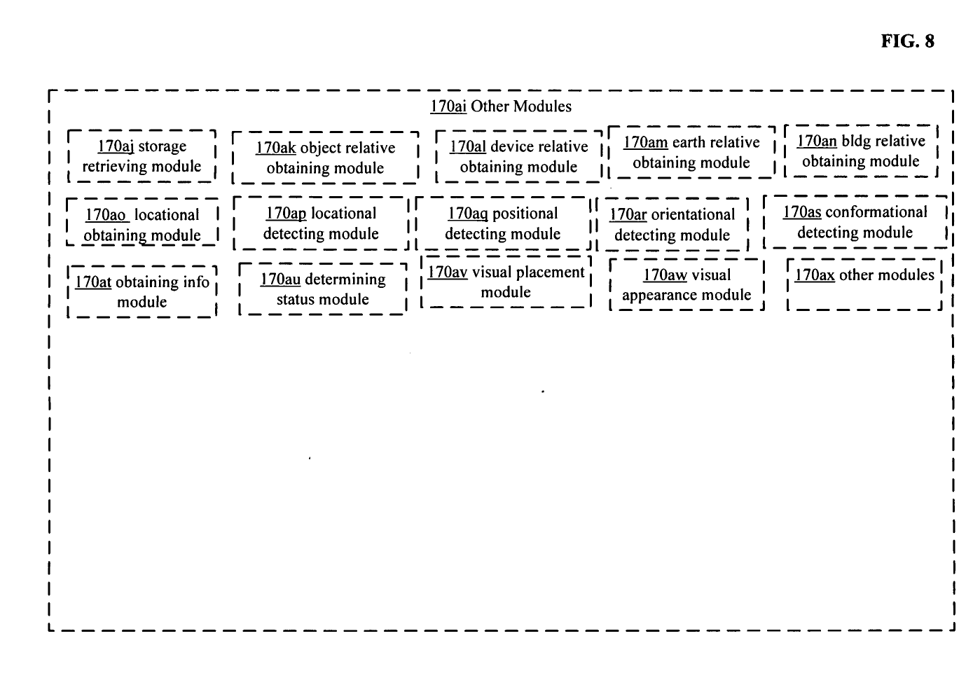

The other modules 170ai is shown in FIG. 8 to further include a storage retrieving module 170aj, an object relative obtaining module 170ak, a device relative obtaining module 170al, an earth relative obtaining module 170am, a building relative obtaining module 170an, a locational obtaining module 170an, a locational detecting module 170ap, a positional detecting module 170aq, an orientational detecting module 170ar, a conformational detecting module 170 as, an obtaining information module 170 at, a determining status module 170au, a visual placement module 170av, a visual appearance module 170aw, and an other modules 170ax.

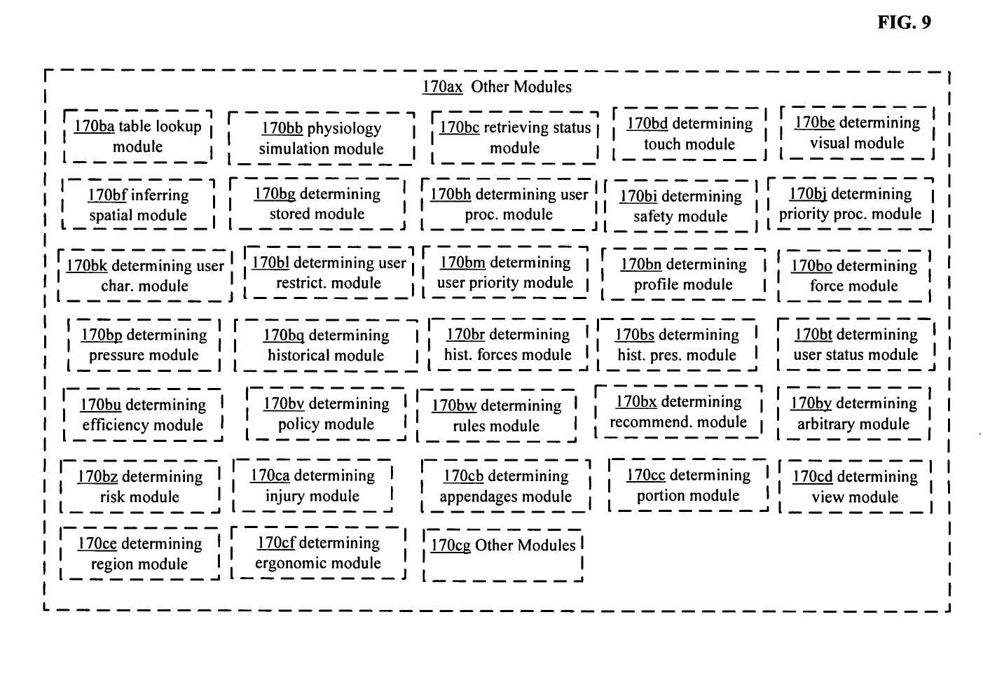

The other modules 170ax is shown in FIG. 9 to further include a table lookup module 170ba, a physiology simulation module 170bb, a retrieving status module 170bc, a determining touch module 170bd, a determining visual module 170ba, an inferring spatial module 170bf, a determining stored module 170bg, a determining subject procedure module 170bh, a determining safety module 170bi, a determining priority procedure module 170bj, a determining subject characteristics module 170bk, a determining subject restrictions module 170bl, a determining subject priority module 170bm, a determining profile module 170bn, a determining force module 170bo, a determining pressure module 170bp, a determining historical module 170bq, a determining historical forces module 170br, a determining historical pressures module 170bs, a determining subject status module 170bt, a determining efficiency module 170bu, a determining policy module 170bv, a determining rules module 170bw, a determining recommendation module 170bx, a determining arbitrary module 170by, a determining risk module 170bz, a determining injury module 170ca, a determining appendages module 170cb, a determining portion module 170cc, a determining view module 170cd, a determining region module 170ce, a determining ergonomic module 170cf, and an other modules 170cg.

An exemplary version of the object 12 is shown in FIG. 10 to optionally include the advisory output 104, the communication unit 112, an exemplary version of the sensors 108, and object functions 172. The sensors 108 optionally include a strain sensor 108a, a stress sensor 108b, an optical sensor 108c, a surface sensor 108d, a force sensor 108e, a gyroscopic sensor 108f, a GPS sensor 108g, an RFID sensor 108h, a inclinometer sensor 108i, an accelerometer sensor 108j, an inertial sensor 1108k, a contact sensor 108l, a pressure sensor 108m, a display sensor 108n.

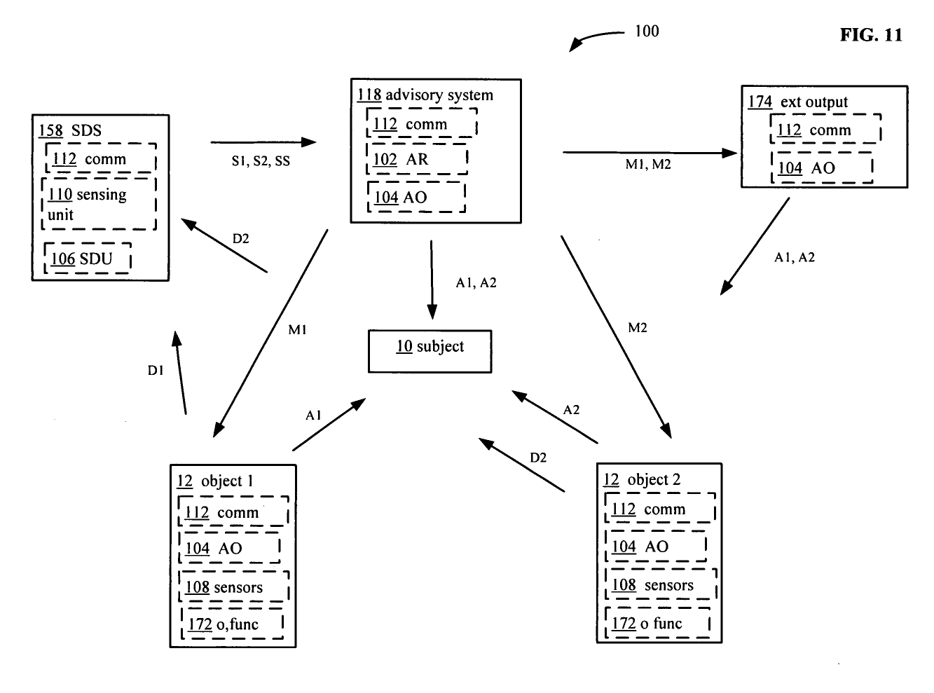

An exemplary configuration of the system 100 is shown in FIG. 11 to include an exemplary versions of the status determination system 158, the advisory system 118, and with two instances of the object 12. The two instances of the object 12 are depicted as “object 1” and “object 2,” respectively. The exemplary configuration is shown to also include an external output 174 that includes the communication unit 112 and the advisory output 104.

As shown in FIG. 11, the status determination system 158 can receive postural influencer status information D1 and D2 as acquired by the sensors 108 of the objects 12, namely, object 1 and object 2, respectively. The postural influencer status information D1 and D2 are acquired by one or more of the sensors 108 of the respective one of the objects 12 and sent to the status determination system 158 by the respective one of the communication unit 112 of the objects. Once the status determination system 158 receives the postural influencer status information D1 and D2, the status determination unit 106, better shown in FIG. 6, uses the control unit 160 to direct determination of status of the objects 12 and the subject 10 through a combined use of the determination engine 167, the storage unit 168, the interface 169, and the modules 170 depending upon the circumstances involved. Status of the subject 10 and the objects 12 can include their spatial status including positional, locational, orientational, and conformational status. In particular, physical status of the subject 10 is of interest since advisories can be subsequently generated to adjust such physical status. Advisories can contain information to also guide adjustment of physical status of the objects 12, such as location, since this can influence the physical status of the subject 10, such as through requiring the subject to view or touch the objects.

Continuing on with FIG. 11, alternatively or in conjunction with receiving the postural influencer status information D1 and D2 from the objects 12, the status determination system 158 can use the sensing unit 110 to acquire information regarding physical status of the objects without necessarily requiring use of the sensors 108 found with the objects. The postural influencer status information acquired by the sensing unit 110 can be sent to the status determination unit 106 through the communication unit 112 for subsequent determination of physical status of the subject 10 and the objects 12.

For the configuration depicted in FIG. 11, once determined, the postural influencer status information SS of the subject 10 of the objects 12 and the postural influencer status information S1 for the object 1 and the postural influencer status information S2 for the object 2 is sent by the communication unit 112 of the status determination system 158 to the communication unit 112 of the advisory system 118. The advisory system 118 then uses this postural influencer status information in conjunction with information and/or algorithms and/or other information processing of the advisory resource unit 102 to generate advisory based content to be included in messages labeled M1 and M2 to be sent to the communication units of the objects 12 to be used by the advisory outputs 104 found in the objects, to the communication units of the external output 174 to be used by the advisory output found in the external output, and/or to be used by the advisory output internal to the advisory system.

If the advisory output 104 of the object 12 (1) is used, it will send an advisory (labeled as A1) to the subject 10 in one or more physical forms (such as light, audio, video, vibration, electromagnetic, textual and/or another indicator or media) directly to the subject or to be observed indirectly by the subject. If the advisory output 104 of the object 12 (2) is used, it will send an advisory (labeled as A2) to the subject 10 in one or more physical forms (such as light, audio, video, vibration, electromagnetic, textual and/or another indicator or media) directly to the subject or to be observed indirectly by the subject. If the advisory output 104 of the external output 174 is used, it will send advisories (labeled as A1 and A2) in one or more physical forms (such as light, audio, video, vibration, electromagnetic, textual and/or another indicator or media) directly to the subject 10 or to be observed indirectly by the subject. If the advisory output 104 of the advisory system 118 is used, it will send advisories (labeled as A1 and A2) in one or more physical forms (such as light, audio, video, vibration, electromagnetic, textual and/or another indicator or media) directly to the subject 10 or to be observed indirectly by the subject. As discussed, an exemplary intent of the advisories is to inform the subject 10 of an alternative configuration for the objects 12 that would allow, encourage, or otherwise support a change in the physical status, such as the posture, of the subject.

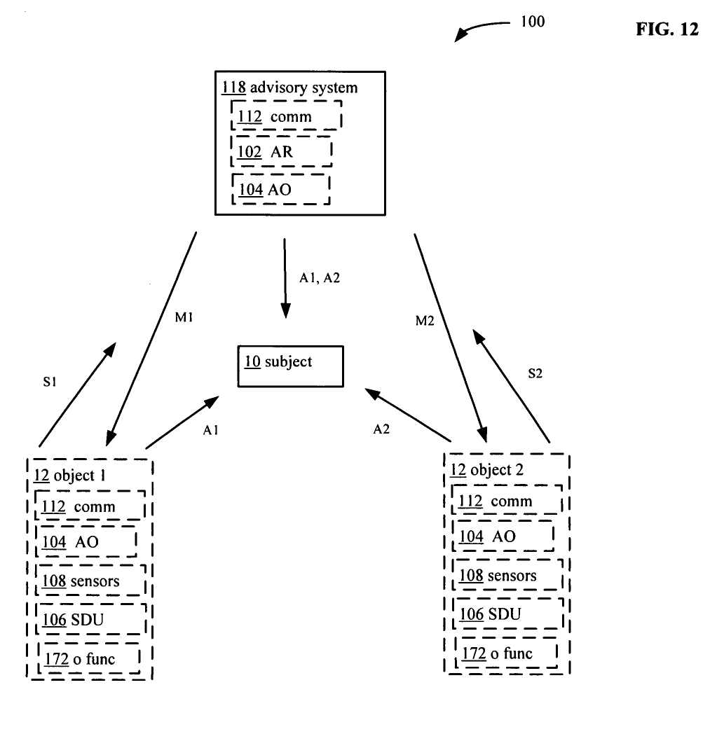

An exemplary alternative configuration for the system 100 is shown in FIG. 12 to include an advisory system 118 and versions of the objects 12 that include the status determination unit 106. Each of the objects 12 are consequently able to determine their physical status through use of the status determination unit from information collected by the one or more sensors 108 found in each of the objects. The postural influencer status information is shown being sent from the objects 12 (labeled as S1 and S2 for that being sent from the object 1 and object 2, respectively) to the advisory system 118. In implementations of the advisory system 118 where an explicit physical status of the subject 10 is not received, the advisory system can infer the physical status of the subject 10 from the physical status received of the objects 12. Instances of the advisory output 104 are found in the advisory system 118 and/or the objects 12 so that the advisories A1 and A2 are sent from the advisory system and/or the objects to the subject 10.

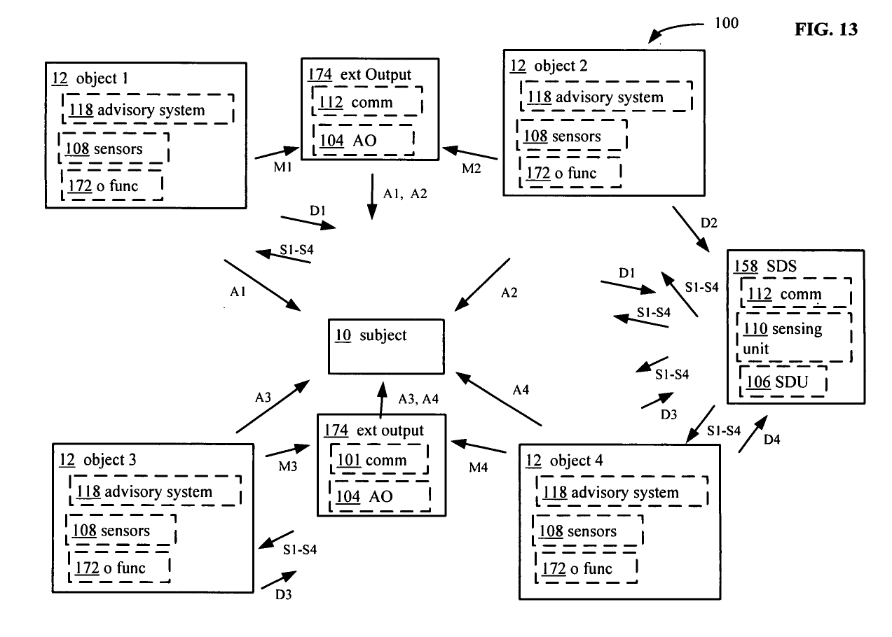

An exemplary alternative configuration for the system 100 is shown in FIG. 13 to include the status determination system 158, two instances of the external output 174, and four instances of the objects 12, which include the advisory system 118. With this configuration, some implementations of the objects 12 can send postural influencer status information D1-D4 as acquired by the sensors 108 found in the objects 12 to the status determination system 158. Alternatively, or in conjunction with the sensors 108 on the objects 12, the sensing unit 110 of the status determination system 158 can acquire information regarding physical status of the objects 12.

Based upon the acquired information of the physical status of the objects 12, the status determination system 158 determines postural influencer status information S1-S4 of the objects 12 (S1-S4 for object 1-object 4, respectively). In some alternatives, all of the postural influencer status information S1-S4 is sent by the status determination system 158 to each of the objects 12 whereas in other implementations different portions are sent to different objects. The advisory system 118 of each of the objects 12 uses the received physical status to determine and to send advisory information either to its respective advisory output 104 or to one of the external outputs 174 as messages M1-M4. In some implementations, the advisory system 118 will infer physical status for the subject 10 based upon the received physical status for the objects 12. Upon receipt of the messages M1-M4, each of the advisory outputs 104 transmits a respective one of the messages M1-M4 to the subject 10. As is evident by the configurations depicted in the Figures, such as FIGS. 11-13, various combinations may exist wherein one or more of the various entities involved such as the status determination system 158 and/or the advisory system 118, and/or external output 174 could be separated from each other and/or the subjects 10 and objects 12 by great distances in accordance with practicality and technology such as including being located in different countries around the world. It should also be understood that in general in order to determine some sort of advisory information based upon some status information, the determiner of the advisory information somehow needs to obtain the status information.

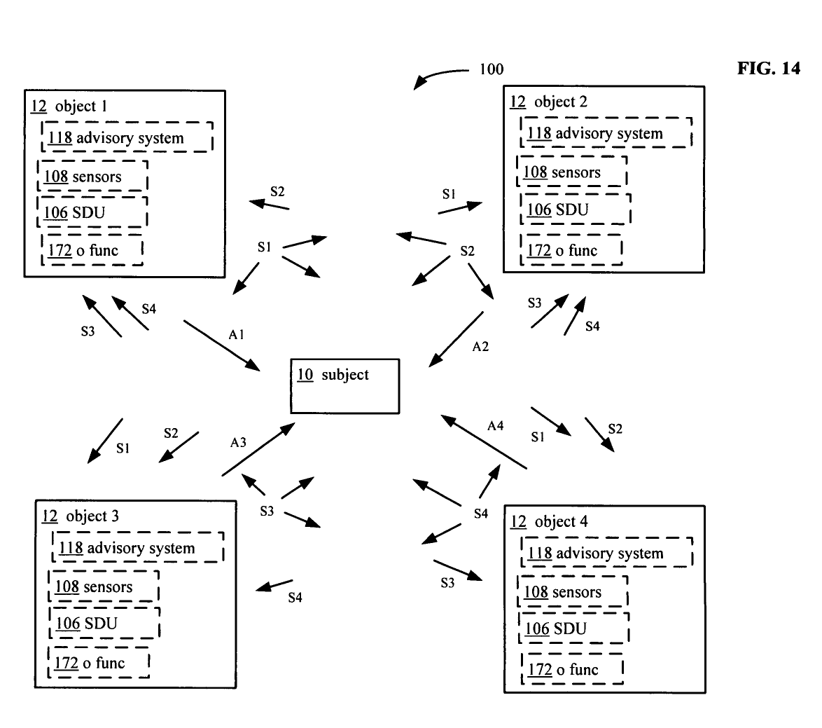

An exemplary alternative configuration for the system 100 is shown in FIG. 14 to include four of the objects 12. Each of the objects 12 includes the status determination unit 106, the sensors 108, and the advisory system 118. Each of the objects 12 obtains postural influencer status information through its instance of the sensors 108 to be used by its instance of the status determination unit 106 to determine physical status of the object. Once determined, the postural influencer status information (S1-S4) of each the objects 12 is shared with all of the objects 12, but in other implementations need not be shared with all of the objects. The advisory system 118 of each of the objects 12 uses the physical status determined by the status determination unit 106 of the object and the physical status received by the object to generate and to send an advisory (A1-A4) from the object to the subject 10.

The various components of the system 100 with implementations including the advisory resource unit 102, the advisory output 104, the status determination unit 106, the sensors 108, the sensing unit 110, and the communication unit 112 and their sub-components and the other exemplary entities depicted may be embodied by hardware, software and/or firmware. For example, in some implementations the system 100 including the advisory resource unit 102, the advisory output 104, the status determination unit 106, the sensors 108, the sensing unit 110, and the communication unit 112 may be implemented with a processor (e.g., microprocessor, controller, and so forth) executing computer readable instructions (e.g., computer program product) stored in a storage medium (e.g., volatile or non-volatile memory) such as a signal-bearing medium. Alternatively, hardware such as application specific integrated circuit (ASIC) may be employed in order to implement such modules in some alternative implementations.

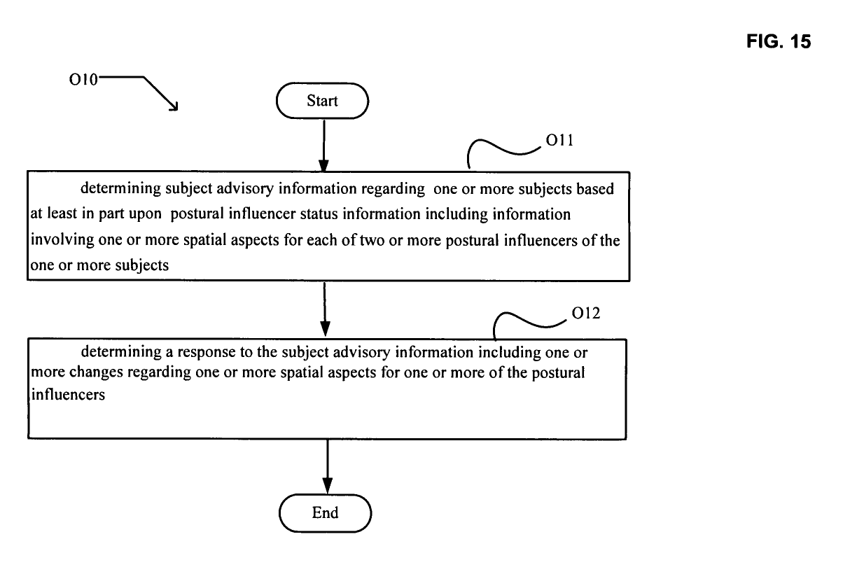

FIG. 15

An operational flow O10 as shown in FIG. 15 represents example operations related to obtaining postural influencer status information, determining subject status information, and determining subject advisory information. In cases where the operational flows involve subjects and devices, as discussed above, in some implementations, the objects 12 can be devices and the subjects 10 can be subjects of the devices. FIG. 15 and those figures that follow may have various examples of operational flows, and explanation may be provided with respect to the above-described examples of FIGS. 1-14 and/or with respect to other examples and contexts. Nonetheless, it should be understood that the operational flows may be executed in a number of other environments and contexts, and/or in modified versions of FIGS. 1-14. Furthermore, although the various operational flows are presented in the sequence(s) illustrated, it should be understood that the various operations may be performed in other orders than those which are illustrated, or may be performed concurrently.

In FIG. 15 and those figures that follow, various operations may be depicted in a box-within-a-box manner. Such depictions may indicate that an operation in an internal box may comprise an optional exemplary implementation of the operational step illustrated in one or more external boxes. However, it should be understood that internal box operations may be viewed as independent operations separate from any associated external boxes and may be performed in any sequence with respect to all other illustrated operations, or may be performed concurrently.

The operational flow O10 may then move to operation O11, where determining subject advisory information regarding one or more subjects based at least in part upon postural influencer status information including information involving one or more spatial aspects for each of two or more postural influencers of the one or more subjects may be executed by, for example, the advisory resource unit 102 of the advisory system 118 of FIG. 3. An exemplary implementation may include, the determining advisory module 120q of FIG. 4 directing the advisory resource unit 102 including to receive the postural influencer status information from the status determination unit 106. As depicted in various Figures, the advisory resource unit 102 can be located in various entities including in a standalone version of the advisory system 118 (e.g. see FIG. 3) or in a version of the advisory system included in the object 12 (e.g. see FIG. 13) and the status determination unit can be located in various entities including the status determination system 158 (e.g. see FIG. 11) or in the objects 12 (e.g. see FIG. 14) so that some implementations include the status determination unit sending the postural influencer status information from the communication unit 112 of the status determination system 158 to the communication unit 112 of the advisory system and other implementations include the status determination unit sending the postural influencer status information to the advisory system internally within each of the objects. Once the postural influencer status information is received, the control unit 122 and the storage unit 130 (including in some implementations the guidelines 132) of the advisory resource unit 102 can determine subject advisory information. In some implementations, the subject advisory information is determined by the control unit 122 looking up various portions of the guidelines 132 contained in the storage unit 130 based upon the postural influencer status information. For example, the postural influencer status information may include locational or positional information for the objects 12 such as those objects depicted in FIG. 2. As an example, the control unit 122 may look up in the storage unit 130 portions of the guidelines associated with this information depicted in FIG. 2 to determine subject advisory information that would inform the subject 10 of FIG. 2 that the subject has been in a posture that over time could compromise integrity of a portion of the subject, such as the trapezius muscle or one or more vertebrae of the subject's spinal column. The subject advisory information could further include one or more suggestions regarding modifications to the existing posture of the subject 10 that may be implemented by repositioning one or more of the objects 12 so that the subject 10 can still use or otherwise interact with the objects in a more desired posture thereby alleviating potential ill effects by substituting the present posture of the subject with a more desired posture. In other implementations, the control unit 122 of the advisory resource unit 102 can include generation of subject advisory information through input of the subject status information into a physiological-based simulation model contained in the memory unit 128 of the control unit, which may then advise of suggested changes to the subject status, such as changes in posture. The control unit 122 of the advisory resource unit 102 may then determine suggested modifications to the physical status of the objects 12 (devices) based upon the postural influencer status information for the objects that was received. These suggested modifications can be incorporated into the determined subject advisory information.

The operational flow O10 may then move to operation O12, where determining a response to the subject advisory information including one or more changes regarding one or more spatial aspects for one or more of the postural influencers may be executed by, for example, the status determining system 158 of FIG. 6. An exemplary implementation may include after the subject advisory information is determined, the response determining module ah directing the status determination unit 106 of the status determination system 158 to process postural influencer status information received by the communication unit 112 of the status determination system from one or more of the objects 12 as postural influencers or other of the postural influencers 13 with respect to another postural influencer and/or obtained through one or more of the components of the sensing unit 110 regarding one or more spatial aspects for one or more of the postural influencers. The postural influencer status information is processed by the status determination unit 106 to ascertain a response to the determined status advisory information such as changes in one or more spatial aspects of one or more of the postural influencers as objects 12 and/or other postural influencers 13. For instance, spatial aspects can include location, position, orientation, conformational and/or other spatial aspects relative to other of the objects 12 or other of the postural influencers 13 or relative to other references such as particular portions of a room or other environment of the objects or other postural influencers. Postural influencer status information could be determined through the use of components including the control unit 160 and the determination engine 167 of the status determining unit 106.

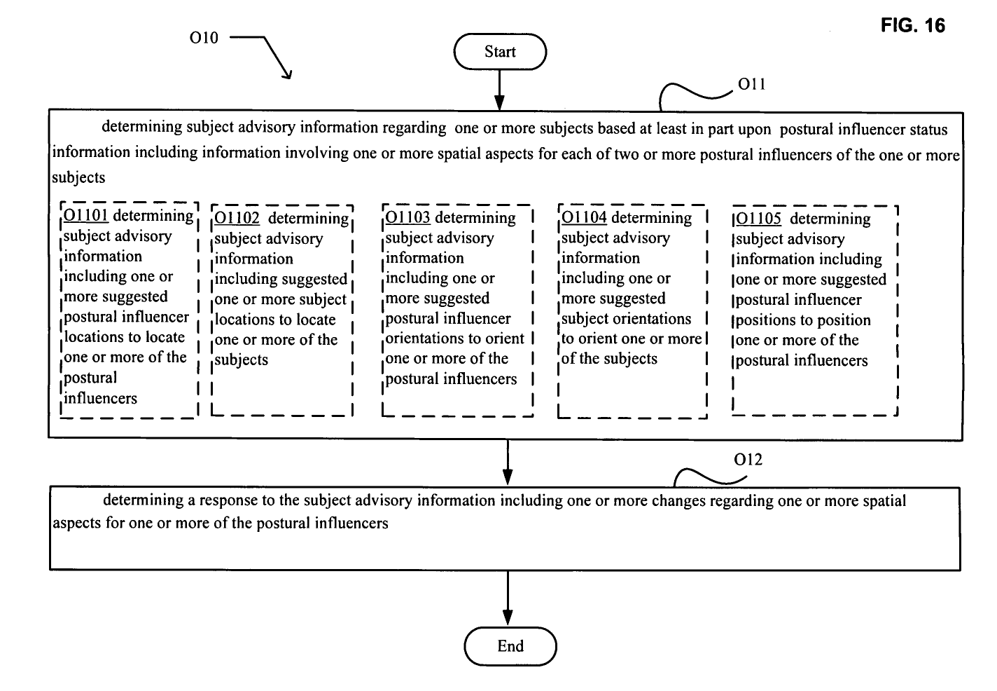

FIG. 16

FIG. 16 illustrates various implementations of the exemplary operation O11 of FIG. 15. In particular, FIG. 16 illustrates example implementations where the operation O11 includes one or more additional operations including, for example, operations O1101, O1102, O1103, O1104, and O1105, which may be executed generally by, in some instances, the status determination unit 106 of the status determination system 158 of FIG. 6.

For instance, in some implementations, the exemplary operation O11 may include the operation of O1101 for determining subject advisory information including one or more suggested postural influencer locations to locate one or more of the postural influencers. An exemplary implementation may include the determining influencer location module 120a of FIG. 4 directing the advisory system 118 including to receive postural influencer status information (such as D1 and D2 as depicted in FIG. 11) for the objects 12 as postural influencers of one or more of the subjects 10 and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested posture or other suggested status for the subject 10. Based upon the suggested status for the subject 10 and the postural influencer status information regarding the objects 12, the control 122 can run an algorithm contained in the memory 128 of the advisory resource unit 102 to generate one or more suggested locations that one or more of the objects could be moved to in order to allow the posture or other status of the subject to be changed as advised. As a result, the advisory resource unit 102 can perform determining subject advisory information including one or more suggested postural influencer locations to locate one or more of the objects 12 as postural influencers.

For instance, in some implementations, the exemplary operation O11 may include the operation of O1102 for determining subject advisory information including suggested one or more subject locations to locate one or more of the subjects. An exemplary implementation may include the determining subject locations module 120b of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested posture or other suggested status for the subject 10. Based upon the suggested status for the subject 10 and the postural influencer status information regarding the objects 12 as postural influencers, the control 122 can run an algorithm contained in the memory 128 of the advisory resource unit 102 to generate one or more suggested locations that the subject could be moved to in order to allow the posture or other status of the subject to be changed as advised. As a result, the advisory resource unit 102 can perform determining subject advisory information including one or more suggested subject locations to locate one or more of the subjects 10.

For instance, in some implementations, the exemplary operation O11 may include the operation of O1103 for determining subject advisory information including one or more suggested postural influencer orientations to orient one or more of the postural influencers. An exemplary implementation may include the determining influencer orientation module 120c of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested posture or other suggested status for the subject 10. Based upon the suggested status for the subject 10 and the postural influencer status information regarding the objects 12 as postural influencers, the control 122 can run an algorithm contained in the memory 128 of the advisory resource unit 102 to generate one or more suggested orientations that one or more of the objects could be oriented at in order to allow the posture or other status of the subject to be changed as advised. As a result, the advisory resource unit 102 can perform determining subject advisory information including one or more suggested postural influencer orientations to orient one or more of the objects 12 as postural influencers.

For instance, in some implementations, the exemplary operation O11 may include the operation of O1104 for determining subject advisory information including one or more suggested subject orientations to orient one or more of the subjects. An exemplary implementation may include the determining subject orientation module 120d of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested posture or other suggested status for the subject 10. Based upon the suggested status for the subject 10 and the postural influencer status information regarding the objects 12 as postural influencers, the control 122 can run an algorithm contained in the memory 128 of the advisory resource unit 102 to generate one or more suggested orientations that the subject as postural influencers could be oriented at in order to allow the posture or other status of the subject to be changed as advised. As a result, the advisory resource unit 102 can perform determining subject advisory information including one or more suggested subject orientations to orient one or more of the subjects 10.

For instance, in some implementations, the exemplary operation O11 may include the operation of O1105 for determining subject advisory information including one or more suggested postural influencer positions to position one or more of the postural influencers. An exemplary implementation may include the determining influencer position module 120e of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested posture or other suggested status for the subject 10. Based upon the suggested status for the subject 10 and the postural influencer status information regarding the objects 12 as postural influencers, the control 122 can run an algorithm contained in the memory 128 of the advisory resource unit 102 to generate one or more suggested positions that one or more of the objects could be moved to order to allow the posture or other status of the subject to be changed as advised. As a result, the advisory resource unit 102 can perform determining subject advisory information including one or more suggested postural influencer positions to position one or more of the objects 12 as postural influencers.

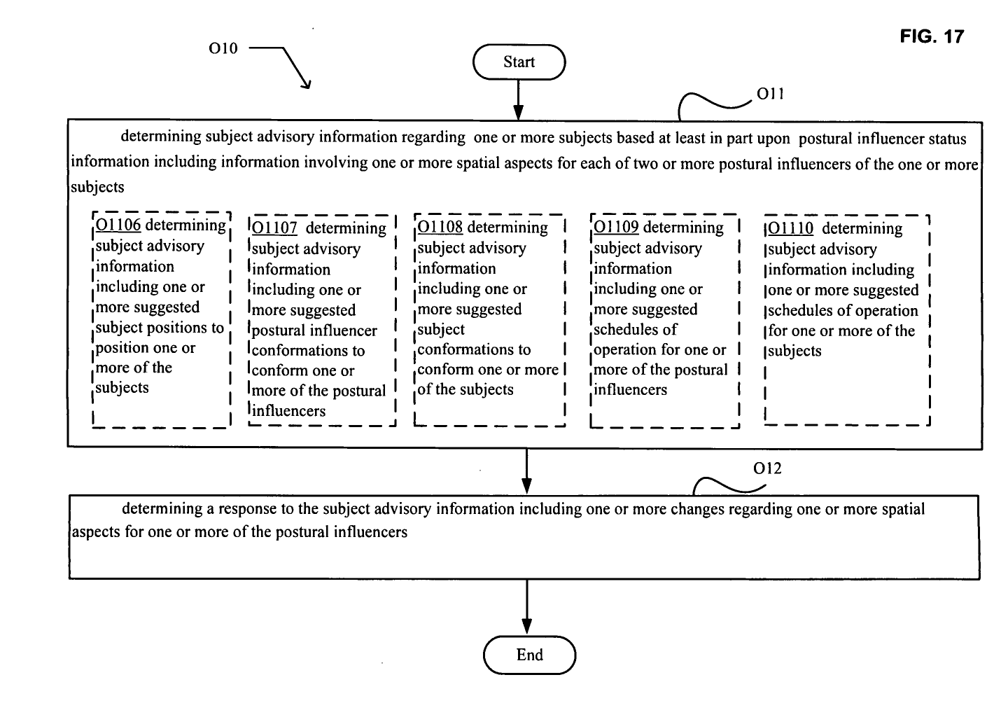

FIG. 17

FIG. 17 illustrates various implementations of the exemplary operation O11 of FIG. 15. In particular, FIG. 17 illustrates example implementations where the operation O11 includes one or more additional operations including, for example, operation O1106, O1107, O1108, O1109, and O1110, which may be executed generally by the advisory system 118 of FIG. 3.

For instance, in some implementations, the exemplary operation O11 may include the operation of O1106 for determining subject advisory information including one or more suggested subject positions to position one or more of the subjects. An exemplary implementation may include the determining subject position module 120f of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested posture or other suggested status for the subject 10. Based upon the suggested status for the subject 10 and the postural influencer status information regarding the objects 12 as postural influencers, the control 122 can run an algorithm contained in the memory 128 of the advisory resource unit 102 to generate one or more suggested positions that the subject as postural influencers could be moved to in order to allow the posture or other status of the subject to be changed as advised. As a result, the advisory resource unit 102 can perform determining subject advisory information including one or more suggested subject positions to position one or more of the subjects 10.

For instance, in some implementations, the exemplary operation O11 may include the operation of O1107 for determining subject advisory information including one or more suggested postural influencer conformations to conform one or more of the postural influencers. An exemplary implementation may include the determining influencer conformation module 120g of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested posture or other suggested status for the subject 10. Based upon the suggested status for the subject 10 and the postural influencer status information regarding the objects 12 as postural influencers, the control 122 can run an algorithm contained in the memory 128 of the advisory resource unit 102 to generate one or more suggested conformations that one or more of the objects could be conformed to in order to allow the posture or other status of the subject to be changed as advised. As a result, the advisory resource unit 102 can perform determining subject advisory information including one or more suggested postural influencer conformations to conform one or more of the objects 12 as postural influencers.

For instance, in some implementations, the exemplary operation O11 may include the operation of O1108 for determining subject advisory information including one or more suggested subject conformations to conform one or more of the subjects. An exemplary implementation may include the determining subject conformation module 120h of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested posture or other suggested status for the subject 10. Based upon the suggested status for the subject 10 and the postural influencer status information regarding the objects 12 as postural influencers, the control 122 can run an algorithm contained in the memory 128 of the advisory resource unit 102 to generate one or more suggested conformations that the subject as postural influencers could be conformed to in order to allow the posture or other status of the subject to be changed as advised. As a result, the advisory resource unit 102 can perform determining subject advisory information including one or more suggested subject conformations to conform one or more of the subjects 10.

For instance, in some implementations, the exemplary operation O11 may include the operation of O1109 for determining subject advisory information including one or more suggested schedules of operation for one or more of the postural influencers. An exemplary implementation may include the determining influencer schedule module 120i of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested schedule to assume a posture or a suggested schedule to assume other suggested status for the subject 10. Based upon the suggested schedule to assume the suggested status for the subject 10 and the postural influencer status information regarding the objects 12 as postural influencers, the control 122 can run an algorithm contained in the memory 128 of the advisory resource unit 102 to generate a suggested schedule to operate the objects to allow for the suggested schedule to assume the suggested posture or other status of the subjects. As a result, the advisory resource unit 102 can perform determining subject advisory information including one or more suggested schedules of operation for one or more of the objects 12 as postural influencers.

For instance, in some implementations, the exemplary operation O11 may include the operation of O1110 for determining subject advisory information including one or more suggested schedules of operation for one or more of the subjects. An exemplary implementation may include the determining subject schedule module 120j of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested schedule to assume a posture or a suggested schedule to assume other suggested status for the subject 10. Based upon the suggested schedule to assume the suggested status for the subject 10 and the postural influencer status information regarding the objects 12 as postural influencers, the control 122 can run an algorithm contained in the memory 128 of the advisory resource unit 102 to generate a suggested schedule of operations for the subject as a subject to allow for the suggested schedule to assume the suggested posture or other status of the subjects. As a result, the advisory resource unit 102 can perform determining subject advisory information including one or more suggested schedules of operation for one or more of the subjects 10.

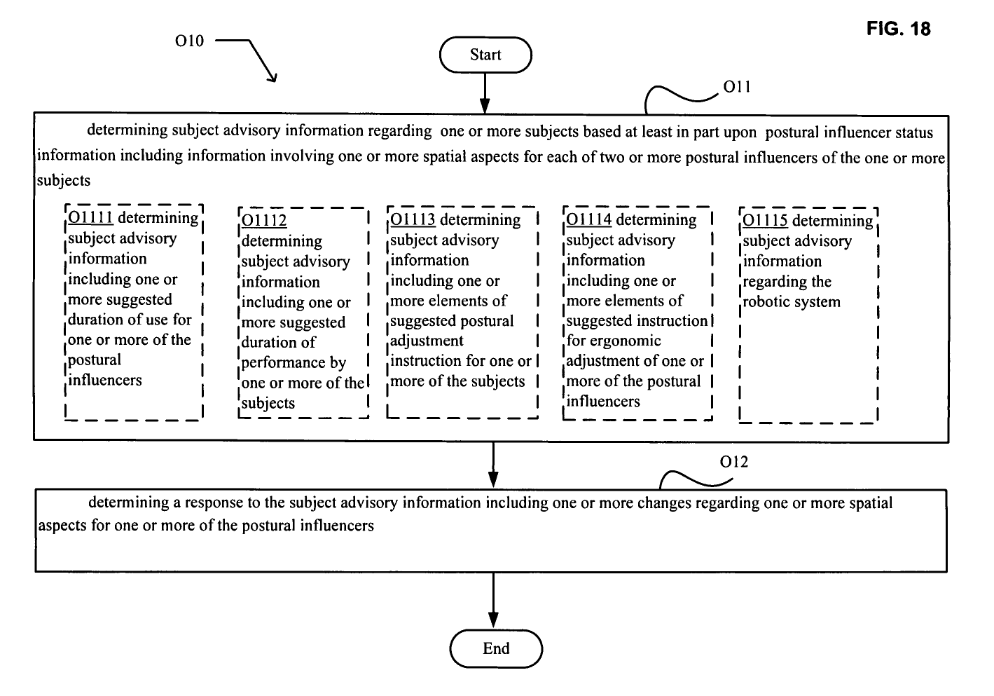

FIG. 18

FIG. 18 illustrates various implementations of the exemplary operation O11 of FIG. 15. In particular, FIG. 18 illustrates example implementations where the operation O11 includes one or more additional operations including, for example, operation O1111, O1112, O1113, O1114, and O1115, which may be executed generally by the advisory system 118 of FIG. 3.

For instance, in some implementations, the exemplary operation O11 may include the operation of O1111 for determining subject advisory information including one or more suggested duration of use for one or more of the postural influencers. An exemplary implementation may include the determining use duration module 120k of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested duration to assume a posture or a suggested schedule to assume other suggested status for the subject 10. Based upon the suggested duration to assume the suggested status for the subject 10 and the postural influencer status information regarding the objects 12 as postural influencers, the control 122 can run an algorithm contained in the memory 128 of the advisory resource unit 102 to generate one or more suggested durations to use the objects to allow for the suggested durations to assume the suggested posture or other status of the subjects. As a result, the advisory resource unit 102 can perform determining subject advisory information including one or more suggested duration of use for one or more of the objects 12 as postural influencers.

For instance, in some implementations, the exemplary operation O11 may include the operation of O1112 for determining subject advisory information including one or more suggested duration of performance by one or more of the subjects. An exemplary implementation may include the determining subject duration module 120l of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate a suggested duration to assume a posture or a suggested schedule to assume other suggested status for the subject 10. Based upon the suggested duration to assume the suggested status for the subject 10 and the postural influencer status information regarding the objects 12 as postural influencers, the control 122 can run an algorithm contained in the memory 128 of the advisory resource unit 102 to generate one or more suggested durations of performance by the subjects. As a result, the advisory resource unit 102 can perform determining subject advisory information including one or more suggested duration of performance by the subject 10 with the objects 12 as postural influencers.

For instance, in some implementations, the exemplary operation O11 may include the operation of O1113 for determining subject advisory information including one or more elements of suggested postural adjustment instruction for one or more of the subjects. An exemplary implementation may include the determining postural adjustment module 120m of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate one or more elements of suggested postural adjustment instruction for the subject 10 to allow for a posture or other status of the subject as advised. As a result, the advisory resource unit 102 can perform determining subject advisory information including one or more elements of suggested postural adjustment instruction for the subject 10 as postural influencers.

For instance, in some implementations, the exemplary operation O11 may include the operation of O1114 for determining subject advisory information including one or more elements of suggested instruction for ergonomic adjustment of one or more of the postural influencers. An exemplary implementation may include the determining ergonomic adjustment module 120n of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate one or more elements of suggested instruction for ergonomic adjustment of one or more of the objects 12 as postural influencers to allow for a posture or other status of the subject 10 as advised. As a result, the advisory resource unit 102 can perform determining subject advisory information including one or more elements of suggested postural adjustment instruction for the subject 10 as postural influencers.

For instance, in some implementations, the exemplary operation O11 may include the operation of O1115 for determining subject advisory information regarding the robotic system. An exemplary implementation may include the determining robotic module 120p of FIG. 4 directing the advisory system 118 including to receive postural influencer status information (such as D1 and D2 as depicted in FIG. 11) for the objects 12 as postural influencers and receiving the subject status information (such as SS as depicted in FIG. 11) for the subject 10 from the status determination unit 106. In implementations, the control 122 of the advisory resource unit 102 can access the memory 128 and/or the storage unit 130 of the advisory resource unit for retrieval or can otherwise use an algorithm contained in the memory to generate advisory information regarding posture or other status of a robotic system as one or more of the subjects 10. As a result, the advisory resource unit 102 can perform determining subject advisory information regarding the robotic system as one or more of the subjects 10.

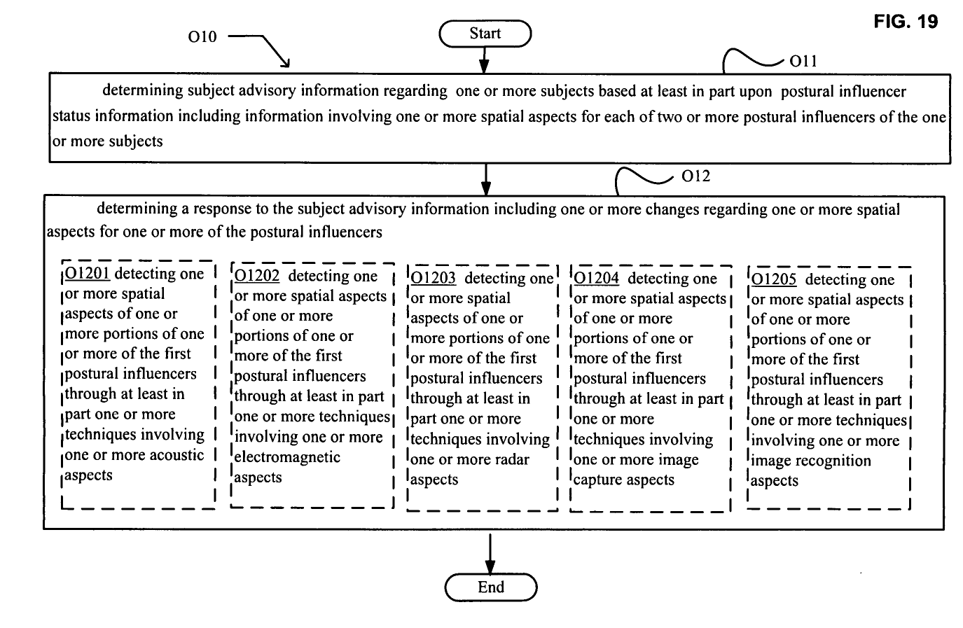

FIG. 19

FIG. 19 illustrates various implementations of the exemplary operation O12 of FIG. 15. In particular, FIG. 15 illustrates example implementations where the operation O12 includes one or more additional operations including, for example, operations O1201, O1202, O1203, O1204, and/or O1205, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O12 may include the operation of O1201 for detecting one or more spatial aspects of one or more portions of one or more of the first postural influencers through at least in part one or more techniques involving one or more acoustic aspects. An exemplary implementation may include the acoustic detecting module 170k of FIG. 7 directing one or more of the acoustic based sensing components 110i of the sensing unit 110 of the status determination system 158 of FIG. 6 to detect one or more spatial aspects of one or more portions of one or more of the objects 12 as first postural influencers of one or more of the subjects 10, which can be devices, through at least in part one or more techniques involving one or more acoustic aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the acoustic based sensing components 110i of the status determination system 158 can be used to detect spatial aspects, such as position, location, orientation, visual placement, visual appearance, and/or conformation of the objects 12.

For instance, in some implementations, the exemplary operation O12 may include the operation of O1202 for detecting one or more spatial aspects of one or more portions of one or more of the first postural influencers through at least in part one or more techniques involving one or more electromagnetic aspects. An exemplary implementation may include the EM detecting module 170l of FIG. 7 directing one or more of the electromagnetic based sensing components 110g of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more electromagnetic aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the electromagnetic based sensing components 110g of the status determination system 158 can be used to detect spatial aspects, such as position, location, orientation, visual placement, visual appearance, and/or conformation of the objects 12.

For instance, in some implementations, the exemplary operation O12 may include the operation of O1203 for detecting one or more spatial aspects of one or more portions of one or more of the first postural influencers through at least in part one or more techniques involving one or more radar aspects. An exemplary implementation may include the radar detecting module 170m of FIG. 7 directing one or more of the radar based sensing components 110k of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more radar aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the radar based sensing components 110k of the status determination system 158 can be used to detect spatial aspects, such as position, location, orientation, visual placement, visual appearance, and/or conformation of the objects 12.

For instance, in some implementations, the exemplary operation O12 may include the operation of O1204 for detecting one or more spatial aspects of one or more portions of one or more of the first postural influencers through at least in part one or more techniques involving one or more image capture aspects. An exemplary implementation may include image capture detecting module 170n of FIG. 7 directing one or more of the image capture based sensing components 110m of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more image capture aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the image capture based sensing components 110m of the status determination system 158 can be used to detect spatial aspects, such as position, location, orientation, visual placement, visual appearance, and/or conformation of the objects 12.

For instance, in some implementations, the exemplary operation O12 may include the operation of O1205 for detecting one or more spatial aspects of one or more portions of one or more of the first postural influencers through at least in part one or more techniques involving one or more image recognition aspects. An exemplary implementation may include the image recognition detecting module 170o of FIG. 7 directing one or more of the image recognition based sensing components 110l of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more image recognition aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the image recognition based sensing components 110l of the status determination system 158 can be used to detect spatial aspects, such as position, location, orientation, visual placement, visual appearance, and/or conformation of the objects 12.

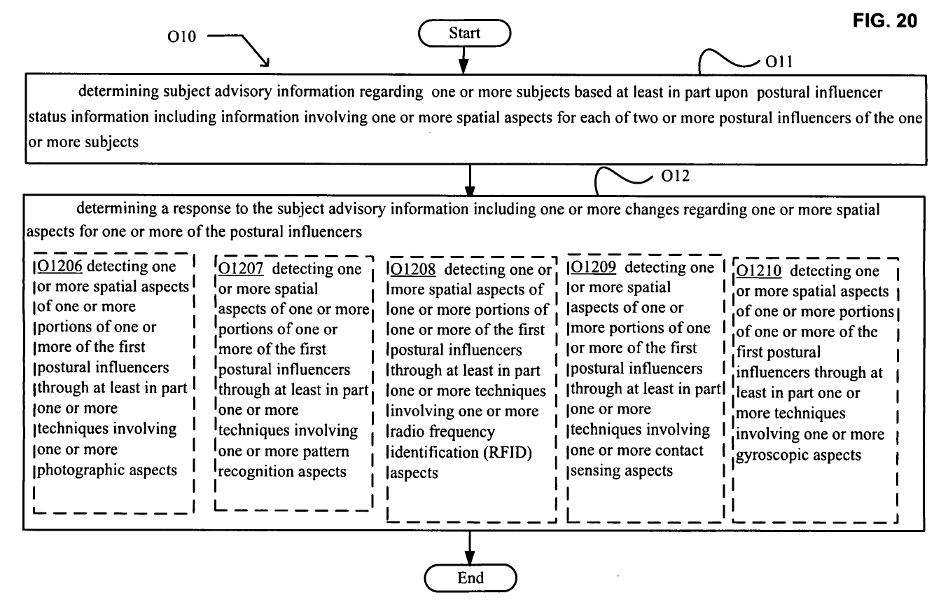

FIG. 20

FIG. 20 illustrates various implementations of the exemplary operation O12 of FIG. 15. In particular, FIG. 20 illustrates example implementations where the operation O12 includes one or more additional operations including, for example, operations O1206, O1207, O1208, O1209, and/or O1210, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O12 may include the operation of O1206 for detecting one or more spatial aspects of one or more portions of one or more of the first postural influencers through at least in part one or more techniques involving one or more photographic aspects. An exemplary implementation may include the photographic detecting module 170p of FIG. 7 directing one or more of the photographic based sensing components 110n of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more photographic aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the photographic based sensing components 110k of the status determination system 158 can be used to detect spatial aspects, such as position, location, orientation, visual placement, visual appearance, and/or conformation of the objects 12.

For instance, in some implementations, the exemplary operation O12 may include the operation of O1207 for detecting one or more spatial aspects of one or more portions of one or more of the first postural influencers through at least in part one or more techniques involving one or more pattern recognition aspects. An exemplary implementation may include the pattern recognition detecting module 170q of FIG. 7 directing one or more of the pattern recognition based sensing components 110e of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more pattern recognition aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the pattern recognition based sensing components 110k of the status determination system 158 can be used to detect spatial aspects, such as position, location, orientation, visual placement, visual appearance, and/or conformation of the objects 12.

For instance, in some implementations, the exemplary operation O12 may include the operation of O1208 for detecting one or more spatial aspects of one or more portions of one or more of the first postural influencers through at least in part one or more techniques involving one or more radio frequency identification (RFID) aspects. An exemplary implementation may include the RFID detecting module 170r of FIG. 7 directing one or more of the RFID based sensing components 110j of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more RFID aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the RFID based sensing components 110k of the status determination system 158 can be used to detect spatial aspects, such as position, location, orientation, visual placement, visual appearance, and/or conformation of the objects 12.

For instance, in some implementations, the exemplary operation O12 may include the operation of O1209 for detecting one or more spatial aspects of one or more portions of one or more of the first postural influencers through at least in part one or more techniques involving one or more contact sensing aspects. An exemplary implementation may include the contact detecting module 170s of FIG. 7 directing one or more of the contact sensors 108l of one or more of the objects 12 as first postural influencers of one or more of the subjects 10 shown in FIG. 10 including to sense contact such as contact made with the objects by the subject 10, such as the subject touching a keyboard device as shown in FIG. 2 to detect one or more spatial aspects of one or more portions of the objects as postural influencers of one or more of the subjects 10. For instance, by sensing contact of the subject 10 (subject) of the object 12 (device), aspects of the orientation of the device with respect to the subject may be detected.

For instance, in some implementations, the exemplary operation O12 may include the operation of O1210 for detecting one or more spatial aspects of one or more portions of one or more of the first postural influencers through at least in part one or more techniques involving one or more gyroscopic aspects. An exemplary implementation may include the gyroscopic detecting module 170t of FIG. 7 directing one or more of the gyroscopic sensors 108f of one or more of the objects 12 as first postural influencers of one or more of the subjects 10 shown in FIG. 10 as postural influencers of one or more of the subjects 10. including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

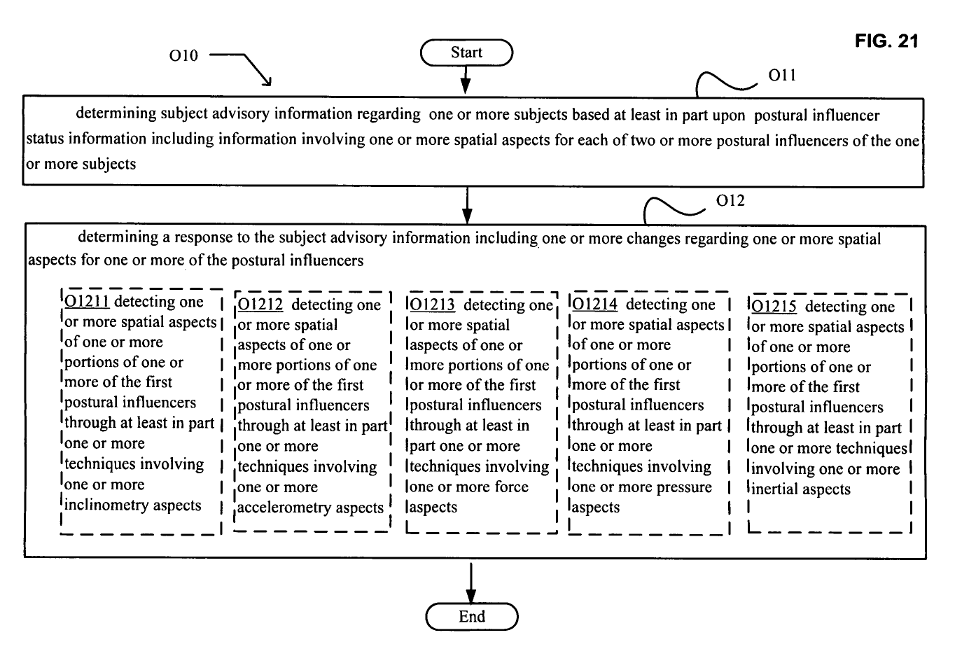

FIG. 21

FIG. 21 illustrates various implementations of the exemplary operation O12 of FIG. 15. In particular, FIG. 21 illustrates example implementations where the operation O12 includes one or more additional operations including, for example, operations O1211, O1212, O1213, O1214, and/or O1215, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O12 may include the operation of O1211 for detecting one or more spatial aspects of one or more portions of one or more of the first postural influencers through at least in part one or more techniques involving one or more inclinometry aspects. An exemplary implementation may include the inclinometry detecting module 170u of FIG. 7 directing one or more of the inclinometers 108i of one or more of the objects 12 as first postural influencers of one or more of the subjects 10 shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

For instance, in some implementations, the exemplary operation O12 may include the operation of O1212 for detecting one or more spatial aspects of one or more portions of one or more of the first postural influencers through at least in part one or more techniques involving one or more accelerometry aspects. An exemplary implementation may include the accelerometry detecting module 170v of FIG. 7 directing one or more of the accelerometers 108j of one or more of the objects 12 as first postural influencers of one or more of the subjects 10 shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

For instance, in some implementations, the exemplary operation O12 may include the operation of O1213 for detecting one or more spatial aspects of one or more portions of one or more of the first postural influencers through at least in part one or more techniques involving one or more force aspects. An exemplary implementation may include the force detecting module 170w of FIG. 7 directing one or more of the force sensors 108e of one or more of the objects 12 as first postural influencers of one or more of the subjects 10 shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

For instance, in some implementations, the exemplary operation O12 may include the operation of O1214 for detecting one or more spatial aspects of one or more portions of one or more of the first postural influencers through at least in part one or more techniques involving one or more pressure aspects. An exemplary implementation may include the pressure detecting module 170x of FIG. 7 directing one or more of the pressure sensors 108m of one or more of the objects 12 as first postural influencers of one or more of the subjects 10. shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

For instance, in some implementations, the exemplary operation O12 may include the operation of O1215 for detecting one or more spatial aspects of one or more portions of one or more of the first postural influencers through at least in part one or more techniques involving one or more inertial aspects. An exemplary implementation may include the inertial detecting module 170y of FIG. 7 directing one or more of the inertial sensors 108k of one or more of the objects 12 as first postural influencers of one or more of the subjects 10 shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

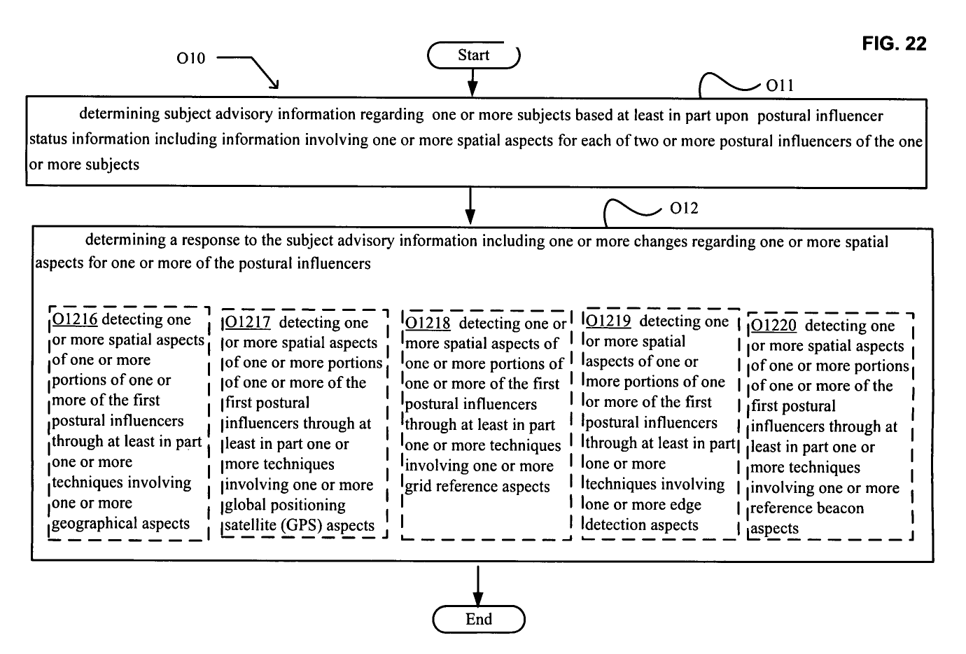

FIG. 22

FIG. 22 illustrates various implementations of the exemplary operation O12 of FIG. 15. In particular, FIG. 22 illustrates example implementations where the operation O12 includes one or more additional operations including, for example, operations O1216, O1217, O1218, O1219, and/or O1220, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O12 may include the operation of O1216 for detecting one or more spatial aspects of one or more portions of one or more of the first postural influencers through at least in part one or more techniques involving one or more geographical aspects. An exemplary implementation may include the geographical detecting module 170z of FIG. 7 directing one or more of the image recognition based sensing components 110l of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more geographical aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the image recognition based sensing components 110l of the status determination system 158 can be used to detect spatial aspects involving geographical aspects, such as position, location, orientation, visual placement, visual appearance, and/or conformation of the objects 12 in relation to a geographical landmark.

For instance, in some implementations, the exemplary operation O12 may include the operation of O1217 for detecting one or more spatial aspects of one or more portions of one or more of the first postural influencers through at least in part one or more techniques involving one or more global positioning satellite (GPS) aspects. An exemplary implementation may include the GPS detecting module 170aa of FIG. 7 directing one or more of the global positioning system (GPS) sensors 108g of one or more of the objects 12 as first postural influencers of one or more of the subjects 10 shown in FIG. 10 including to detect one or more spatial aspects of the one or more portions of the device. Spatial aspects can include location and position as provided by the global positioning system (GPS) to the global positioning system (GPS) sensors 108g of the objects 12 involved and can be sent to the status determination system 158 as transmissions D1 and D2 by the objects as shown in FIG. 11.

For instance, in some implementations, the exemplary operation O12 may include the operation of O1218 for detecting one or more spatial aspects of one or more portions of one or more of the first postural influencers through at least in part one or more techniques involving one or more grid reference aspects. An exemplary implementation may include the grid reference detecting module 170ab of FIG. 7 directing one or more of the grid reference based sensing components 110o of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more grid reference aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the grid reference based sensing components 110o of the status determination system 158 can be used to detect spatial aspects involving grid reference aspects, such as position, location, orientation, visual placement, visual appearance, and/or conformation of the objects 12.

For instance, in some implementations, the exemplary operation O12 may include the operation of O1219 for detecting one or more spatial aspects of one or more portions of one or more of the first postural influencers through at least in part one or more techniques involving one or more edge detection aspects. An exemplary implementation may include the edge detecting module 170ac of FIG. 7 directing one or more of the edge detection based sensing components 110p of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more edge detection aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the edge detection based sensing components 110p of the status determination system 158 can be used to detect spatial aspects involving edge detection aspects, such as position, location, orientation, visual placement, visual appearance, and/or conformation of the objects 12.

For instance, in some implementations, the exemplary operation O12 may include the operation of O1220 for detecting one or more spatial aspects of one or more portions of one or more of the first postural influencers through at least in part one or more techniques involving one or more reference beacon aspects. An exemplary implementation may include the beacon detecting module 170ad of FIG. 7 directing one or more of the reference beacon based sensing components 110q of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more reference beacon aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the reference beacon based sensing components 110q of the status determination system 158 can be used to detect spatial aspects involving reference beacon aspects, such as position, location, orientation, visual placement, visual appearance, and/or conformation of the objects 12.

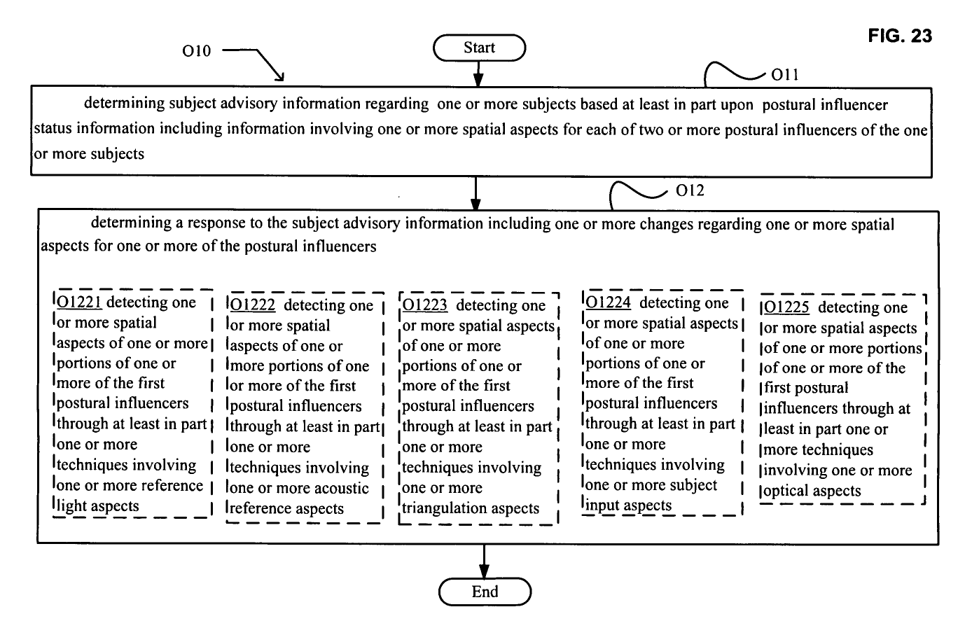

FIG. 23

FIG. 23 illustrates various implementations of the exemplary operation O12 of FIG. 15. In particular, FIG. 23 illustrates example implementations where the operation O12 includes one or more additional operations including, for example, operations O1221, O1222, O1223, O1224, and/or O1225, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O12 may include the operation of O1221 for detecting one or more spatial aspects of one or more portions of one or more of the first postural influencers through at least in part one or more techniques involving one or more reference light aspects. An exemplary implementation may include the reference light detecting module 170ae of FIG. 7 directing one or more of the reference light based sensing components 110r of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more reference light aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the reference light based sensing components 110r of the status determination system 158 can be used to detect spatial aspects involving reference light aspects, such as position, location, orientation, visual placement, visual appearance, and/or conformation of the objects 12.

For instance, in some implementations, the exemplary operation O12 may include the operation of O1222 for detecting one or more spatial aspects of one or more portions of one or more of the first postural influencers through at least in part one or more techniques involving one or more acoustic reference aspects. An exemplary implementation may include the acoustic reference detecting module 170af of FIG. 7 directing one or more of the acoustic reference based sensing components 110s of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more acoustic reference aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the acoustic reference based sensing components 110s of the status determination system 158 can be used to detect spatial aspects involving acoustic reference aspects, such as position, location, orientation, visual placement, visual appearance, and/or conformation of the objects 12.

For instance, in some implementations, the exemplary operation O12 may include the operation of O1223 for detecting one or more spatial aspects of one or more portions of one or more of the first postural influencers through at least in part one or more techniques involving one or more triangulation aspects. An exemplary implementation may include the triangulation detecting module 170ag of FIG. 7 directing one or more of the triangulation based sensing components 110t of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more triangulation aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the triangulation based sensing components 110t of the status determination system 158 can be used to detect spatial aspects involving triangulation aspects, such as position, location, orientation, visual placement, visual appearance, and/or conformation of the objects 12.

For instance, in some implementations, the exemplary operation O12 may include the operation of O1224 for detecting one or more spatial aspects of one or more portions of one or more of the first postural influencers through at least in part one or more techniques involving one or more subject input aspects. An exemplary implementation may include the subject input module 170ah of FIG. 7 directing subject input aspects as detected by one or more of the contact sensors 108l of one or more of the objects 12 as first postural influencers of one or more of the subjects 10 shown in FIG. 10 including to sense contact such as contact made with the object by the subject 10, such as the subject touching a keyboard device as shown in FIG. 2 to detect one or more spatial aspects of one or more portions of the object as a device. For instance, by sensing contact by the subject 10 (subject) as subject input of the object 12 (device), aspects of the orientation of the object with respect to the subject may be detected.

For instance, in some implementations, the exemplary operation O12 may include the operation of O1225 for detecting one or more spatial aspects of one or more portions of one or more of the first postural influencers through at least in part one or more techniques involving one or more optical aspects. An exemplary implementation may include the optical detecting module 170j of FIG. 7 directing one or more of the optical based sensing components 110b of the status determination system 158 of FIG. 6 to detect one or more spatial aspects of one or more portions of one or more of the objects 12 as first postural influencers of one or more of the subjects 10, which can be devices, through at least in part one or more techniques involving one or more optical aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the optical based sensing components 110b of the status determination system 158 can be used to detect spatial aspects, such as position, location, orientation, visual placement, visual appearance, and/or conformation of the objects 12.

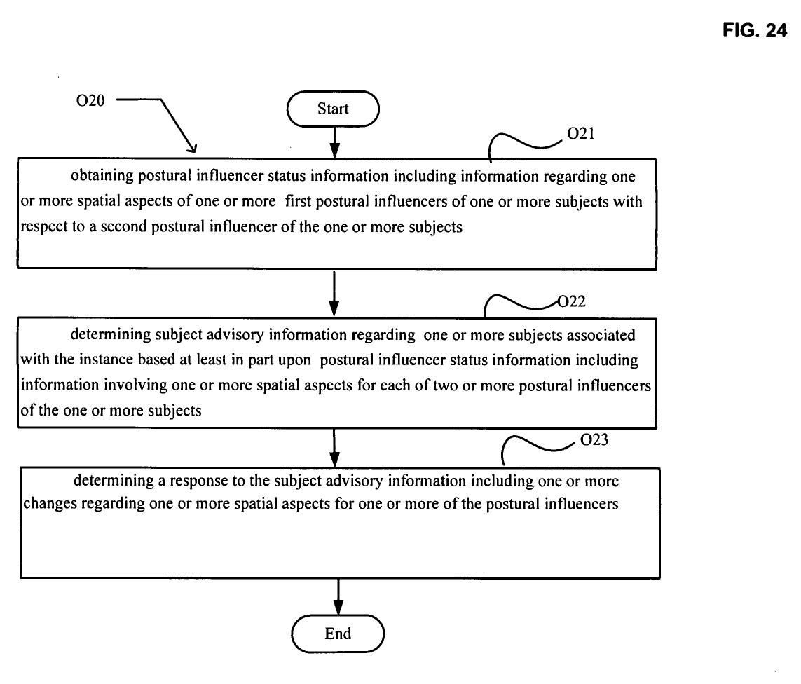

FIG. 24

An operational flow O20 as shown in FIG. 24 represents example operations related to obtaining postural influencer status information, determining subject status information, and determining subject advisory information. In cases where the operational flows involve subjects and devices, as discussed above, in some implementations, the objects 12 can be devices and the subjects 10 can be subjects of the devices. FIG. 24 and those figures that follow may have various examples of operational flows, and explanation may be provided with respect to the above-described examples of FIGS. 1-14 and/or with respect to other examples and contexts. Nonetheless, it should be understood that the operational flows may be executed in a number of other environments and contexts, and/or in modified versions of FIGS. 1-14. Furthermore, although the various operational flows are presented in the sequence(s) illustrated, it should be understood that the various operations may be performed in other orders than those which are illustrated, or may be performed concurrently.

In FIG. 24 and those figures that follow, various operations may be depicted in a box-within-a-box manner. Such depictions may indicate that an operation in an internal box may comprise an optional exemplary implementation of the operational step illustrated in one or more external boxes. However, it should be understood that internal box operations may be viewed as independent operations separate from any associated external boxes and may be performed in any sequence with respect to all other illustrated operations, or may be performed concurrently.

The operational flow O20 may move to operation O21 as shown in FIG. 24, where obtaining postural influencer status information including information regarding one or more spatial aspects of one or more first postural influencers of one or more subjects with respect to a second postural influencer of the one or more subjects may be executed by, for example, the status determining system 158 of FIG. 6. An exemplary implementation may include the obtaining conformation module 170ax of FIG. 8 directing the status determination unit 106 of the status determination system 158 including to process postural influencer status information received by the communication unit 112 of the status determination system from one or more of the objects 12 as first postural influencers with respect to another object a second postural influencer and/or obtained through one or more of the components of the sensing unit 110 to determine subject status information. Subject status information could be determined through the use of components including the control unit 160 and the determination engine 167 of the status determining unit 106 indirectly based upon the postural influencer status information regarding the objects 12 such as the control unit 160 and the determination engine 167 may imply locational, positional, orientational and/or conformational information about one or more subjects based upon related information obtained or determined about the objects 12 involved. For instance, the subject 10 (human subject) of FIG. 2, may have certain locational, positional, orientational, or conformational status characteristics depending upon how the objects 12 (devices) of FIG. 2 are positioned relative to the subject. The subject 10 is depicted in FIG. 2 as viewing the object 12 (display device), which implies certain postural restriction for the subject and holding the object (probe device) to probe the procedure recipient, which implies other postural restriction. As depicted, the subject 10 of FIG. 2 has further requirements for touch and/or verbal interaction with one or more of the objects 12, which further imposes postural restriction for the subject. Various orientations or conformations of one or more of the objects 12 can impose even further postural restriction. Positional, locational, orientational, visual placement, visual appearance, and/or conformational information and possibly other postural influencer status information obtained about the objects 12 of FIG. 2 can be used by the control unit 160 and the determination engine 167 of the status determination unit 106 can imply a certain posture for the subject of FIG. 2 as an example of obtaining postural influencer status information including information regarding one or more spatial aspects of one or more first postural influencers of one or more subjects with respect to a second postural influencer of the one or more subjects. Other implementations of the status determination unit 106 can use postural influencer status information about the subject 10 obtained by the sensing unit 110 of the status determination system 158 of FIG. 6 alone or status of the objects 12 (as described immediately above) for obtaining postural influencer status information including information regarding one or more spatial aspects of one or more first postural influencers of one or more subjects with respect to a second postural influencer of the one or more subjects. For instance, in some implementations, postural influencer status information obtained by one or more components of the sensing unit 110, such as the radar based sensing component 110k, can be used by the status determination unit 106, such as for determining subject status information associated with positional, locational, orientation, and/or conformational information regarding the subject 10 and/or regarding the subject relative to the objects 12.

The operational flow O20 may then move to operation O22, where determining subject advisory information regarding one or more subjects based at least in part upon postural influencer status information including information involving one or more spatial aspects for each of two or more postural influencers of the one or more subjects may be executed by, for example, the advisory resource unit 102 of the advisory system 118 of FIG. 3. An exemplary implementation may include, the determining advisory module 120q of FIG. 4 directing the advisory resource unit 102 including to receive the postural influencer status information from the status determination unit 106. As depicted in various Figures, the advisory resource unit 102 can be located in various entities including in a standalone version of the advisory system 118 (e.g. see FIG. 3) or in a version of the advisory system included in the object 12 (e.g. see FIG. 13) and the status determination unit can be located in various entities including the status determination system 158 (e.g. see FIG. 11) or in the objects 12 (e.g. see FIG. 14) so that some implementations include the status determination unit sending the postural influencer status information from the communication unit 112 of the status determination system 158 to the communication unit 112 of the advisory system and other implementations include the status determination unit sending the postural influencer status information to the advisory system internally within each of the objects. Once the postural influencer status information is received, the control unit 122 and the storage unit 130 (including in some implementations the guidelines 132) of the advisory resource unit 102 can determine subject advisory information. In some implementations, the subject advisory information is determined by the control unit 122 looking up various portions of the guidelines 132 contained in the storage unit 130 based upon the postural influencer status information. For example, the postural influencer status information may include locational or positional information for the objects 12 such as those objects depicted in FIG. 2. As an example, the control unit 122 may look up in the storage unit 130 portions of the guidelines associated with this information depicted in FIG. 2 to determine subject advisory information that would inform the subject 10 of FIG. 2 that the subject has been in a posture that over time could compromise integrity of a portion of the subject, such as the trapezius muscle or one or more vertebrae of the subject's spinal column. The subject advisory information could further include one or more suggestions regarding modifications to the existing posture of the subject 10 that may be implemented by repositioning one or more of the objects 12 so that the subject 10 can still use or otherwise interact with the objects in a more desired posture thereby alleviating potential ill effects by substituting the present posture of the subject with a more desired posture. In other implementations, the control unit 122 of the advisory resource unit 102 can include generation of subject advisory information through input of the subject status information into a physiological-based simulation model contained in the memory unit 128 of the control unit, which may then advise of suggested changes to the subject status, such as changes in posture. The control unit 122 of the advisory resource unit 102 may then determine suggested modifications to the physical status of the objects 12 (devices) based upon the postural influencer status information for the objects that was received. These suggested modifications can be incorporated into the determined subject advisory information.

The operational flow O20 may then move to operation O23, where determining a response to the subject advisory information including one or more changes regarding one or more spatial aspects for one or more of the postural influencers may be executed by, for example, the status determining system 158 of FIG. 6.

An exemplary implementation may include after the subject advisory information is determined, the response determining module ah directing the status determination unit 106 of the status determination system 158 to process postural influencer status information received by the communication unit 112 of the status determination system from one or more of the objects 12 as postural influencers or other of the postural influencers 13 with respect to another postural influencer and/or obtained through one or more of the components of the sensing unit 110 regarding one or more spatial aspects for one or more of the postural influencers. The postural influencer status information is processed by the status determination unit 106 to ascertain a response to the determined status advisory information such as changes in one or more spatial aspects of one or more of the postural influencers as objects 12 and/or other postural influencers 13. For instance, spatial aspects can include location, position, orientation, conformational and/or other spatial aspects relative to other of the objects 12 or other of the postural influencers 13 or relative to other references such as particular portions of a room or other environment of the objects or other postural influencers. Postural influencer status information could be determined through the use of components including the control unit 160 and the determination engine 167 of the status determining unit 106.

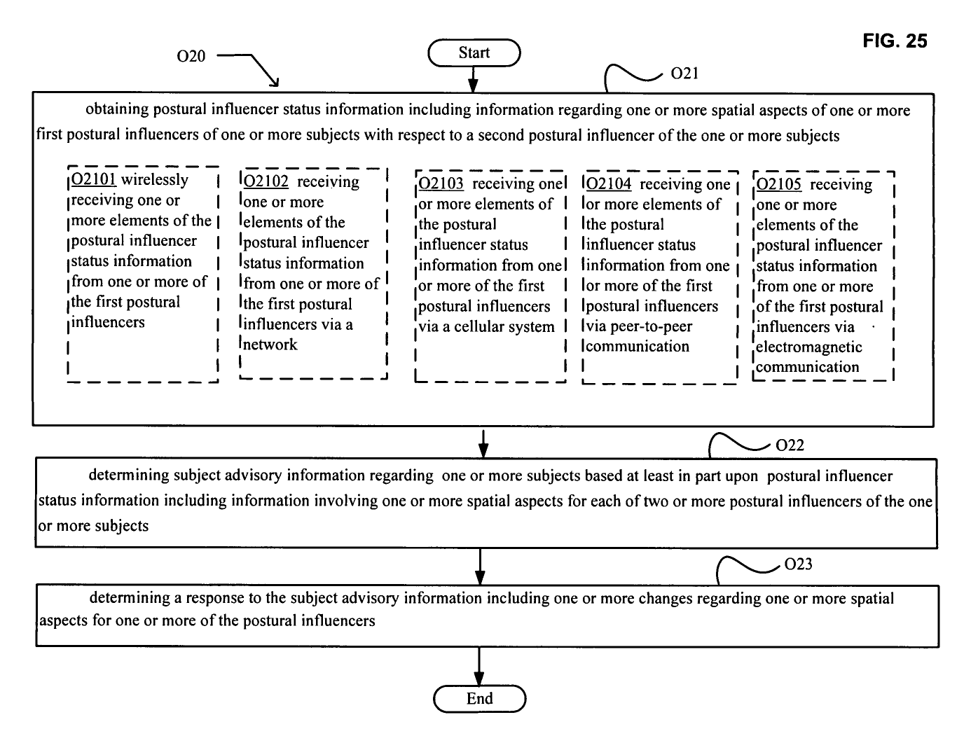

FIG. 25

FIG. 25 illustrates various implementations of the exemplary operation O21 of FIG. 24. In particular, FIG. 25 illustrates example implementations where the operation O21 includes one or more additional operations including, for example, operations O2101, O2102, O2103, O2104, and/or O2105, which may be executed generally by, in some instances, the status determination unit 106 of the status determination system 158 of FIG. 6.

For instance, in some implementations, the exemplary operation O21 may include the operation of O2101 for wirelessly receiving one or more elements of the postural influencer status information from one or more of the first postural influencers. An exemplary implementation may include the wireless receiving module 170a of FIG. 7 directing one or more of the wireless transceiver components 156b of the communication unit 112 of the status determination system 158 of FIG. 6 to receive wireless transmissions from each wireless transceiver component 156b of FIG. 10 of the communication unit 112 of ore or more of the objects 12 as first postural influencers of one or more of the subjects 10. For example, in some implementations, the transmission D1 from object 1 carrying postural influencer status information regarding object 1 and the transmission D2 from object 2 carrying postural influencer status information about object 2 to the status determination system 158, as shown in FIG. 11, can be sent and received by the wireless transceiver components 156b of the objects 12 and the status determination system 158, respectively, as wireless transmissions.

For instance, in some implementations, the exemplary operation O21 may include the operation of O2102 for receiving one or more elements of the postural influencer status information from one or more of the first postural influencers via a network. An exemplary implementation may include the network receiving module 170b of FIG. 7 directing one or more of the network transceiver components 156a of the communication unit 112 of the status determination system 158 of FIG. 6 to receive network transmissions from each network transceiver component 156a of FIG. 10 of the communication unit 112 of one or more of the objects 12 as first postural influencers of one or more of the subjects 10. For example, in some implementations, the transmission D1 from object 1 carrying postural influencer status information regarding object 1 and the transmission D2 from object 2 carrying postural influencer status information about object 2 to the status determination system 158, as shown in FIG. 11, can be sent and received by the network transceiver components 156a of the objects 12 and the status determination system 158, respectively, as network transmissions.

For instance, in some implementations, the exemplary operation O21 may include the operation of O2103 for receiving one or more elements of the postural influencer status information from one or more of the first postural influencers via a cellular system. An exemplary implementation may include the cellular receiving module 170c of FIG. 7 directing one or more of the cellular transceiver components 156c of the communication unit 112 of the status determination system 158 of FIG. 6 to receive cellular transmissions from each cellular transceiver component 156a of FIG. 10 of the communication unit 112 of one or more of the objects 12 as first postural influencers of one or more of the subjects 10. For example, in some implementations, the transmission D1 from object 1 carrying postural influencer status information regarding object 1 and the transmission D2 from object 2 carrying postural influencer status information about object 2 to the status determination system 158, as shown in FIG. 11, can be sent and received by the cellular transceiver components 156c of the objects 12 and the status determination system 158, respectively, as cellular transmissions.

For instance, in some implementations, the exemplary operation O21 may include the operation of O2104 for receiving one or more elements of the postural influencer status information from one or more of the first postural influencers via peer-to-peer communication. An exemplary implementation may include the peer-to-peer receiving module 170d of FIG. 7 directing one or more of the peer-to-peer transceiver components 156d of the communication unit 112 of the status determination system 158 of FIG. 6 to receive peer-to-peer transmissions from each peer-to-peer transceiver component 156d of FIG. 10 of the communication unit 112 of one or more the objects 12 as first postural influencers of one or more of the subjects 10. For example, in some implementations, the transmission D1 from object 1 carrying postural influencer status information regarding object 1 and the transmission D2 from object 2 carrying postural influencer status information about object 2 to the status determination system 158, as shown in FIG. 11, can be sent and received by the peer-to-peer transceiver components 156d of the objects 12 and the status determination system 158, respectively, as peer-to-peer transmissions.

For instance, in some implementations, the exemplary operation O21 may include the operation of O2105 for receiving one or more elements of the postural influencer status information from one or more of the first postural influencers via electromagnetic communication. An exemplary implementation may include the EM receiving module 170e of FIG. 7 directing one or more of the electromagnetic communication transceiver components 156e of the communication unit 112 of the status determination system 158 of FIG. 6 to receive electromagnetic communication transmissions from each electromagnetic communication transceiver component 156a of FIG. 10 of the communication unit 112 of one or more of the objects 12 as first postural influencers of one or more of the subjects 10. For example, in some implementations, the transmission D1 from object 1 carrying postural influencer status information regarding object 1 and the transmission D2 from object 2 carrying postural influencer status information about object 2 to the status determination system 158, as shown in FIG. 11, can be sent and received by the electromagnetic communication transceiver components 156c of the objects 12 and the status determination system 158, respectively, as electromagnetic communication transmissions.

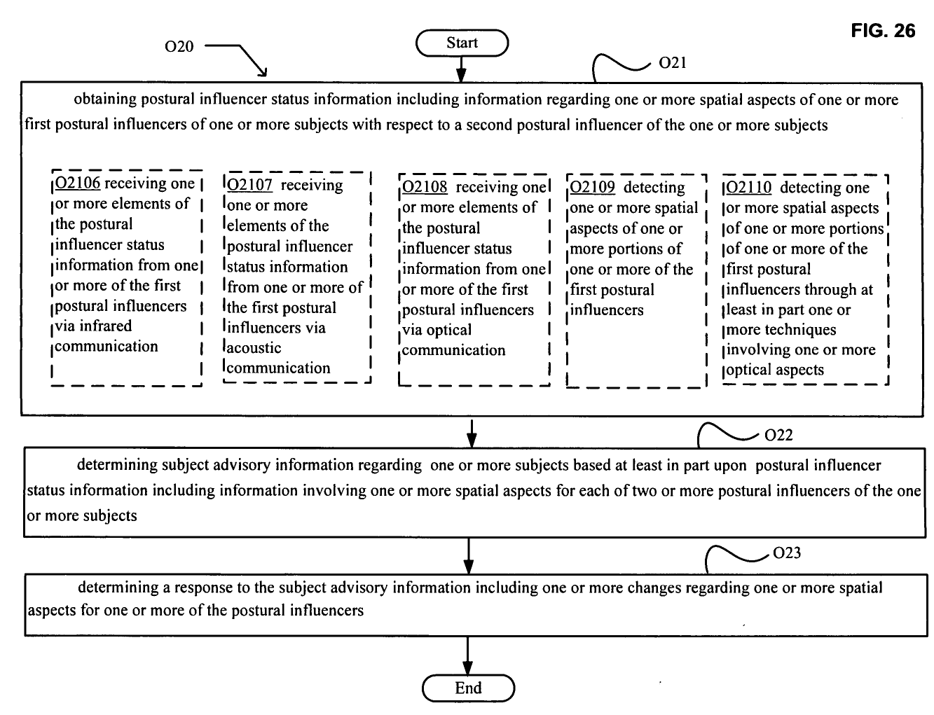

FIG. 26

FIG. 26 illustrates various implementations of the exemplary operation O21 of FIG. 2. In particular, FIG. 26 illustrates example implementations where the operation O21 includes one or more additional operations including, for example, operations O2106, O2107, O2108, O2109, and/or O2110, which may be executed generally by, in some instances, one or more of the transceiver components 156 of the communication unit 112 or one or more sensing components of the sensing unit 110 of the status determination system 158 of FIG. 6.

For instance, in some implementations, the exemplary operation O21 may include the operation of O2106 for receiving one or more elements of the postural influencer status information from one or more of the first postural influencers via infrared communication. An exemplary implementation may include the infrared receiving module 170f of FIG. 7 directing one or more of the infrared transceiver components 156f of the communication unit 112 of the status determination system 158 of FIG. 6 to receive infrared transmissions from each infrared transceiver component 156f of FIG. 10 of the communication unit 112 of one or more of the objects 12 as first postural influencers of one or more of the subjects 10. For example, in some implementations, the transmission D1 from object 1 carrying postural influencer status information regarding object 1 and the transmission D2 from object 2 carrying postural influencer status information about object 2 to the status determination system 158, as shown in FIG. 11, can be sent and received by the infrared transceiver components 156c of the objects 12 and the status determination system 158, respectively, as infrared transmissions.

For instance, in some implementations, the exemplary operation O21 may include the operation of O2107 for receiving one or more elements of the postural influencer status information from one or more of the first postural influencers via acoustic communication. An exemplary implementation may include the acoustic receiving module 170g of FIG. 7 directing one or more of the acoustic transceiver components 156g of the communication unit 112 of the status determination system 158 of FIG. 6 to receive acoustic transmissions from each acoustic transceiver component 156g of FIG. 10 of the communication unit 112 of one or more of the objects 12 as first postural influencers of one or more of the subjects 10. For example, in some implementations, the transmission D1 from object 1 carrying postural influencer status information regarding object 1 and the transmission D2 from object 2 carrying postural influencer status information about object 2 to the status determination system 158, as shown in FIG. 11, can be sent and received by the acoustic transceiver components 156g of the objects 12 and the status determination system 158, respectively, as acoustic transmissions.

For instance, in some implementations, the exemplary operation O21 may include the operation of O2108 for receiving one or more elements of the postural influencer status information from one or more of the first postural influencers via optical communication. An exemplary implementation may include the optical receiving module 170h of FIG. 7 directing one or more of the optical transceiver components 156h of the communication unit 112 of the status determination system 158 of FIG. 6 to receive optical transmissions from each optical transceiver component 156h of FIG. 10 of the communication unit 112 of one or more of the objects 12 as first postural influencers of one or more of the subjects 10. For example, in some implementations, the transmission D1 from object 1 carrying postural influencer status information regarding object 1 and the transmission D2 from object 2 carrying postural influencer status information about object 2 to the status determination system 158, as shown in FIG. 11, can be sent and received by the optical transceiver components 156h of the objects 12 and the status determination system 158, respectively, as optical transmissions.

For instance, in some implementations, the exemplary operation O21 may include the operation of O2109 for detecting one or more spatial aspects of one or more portions of one or more of the first postural influencers. the detecting module 170i of FIG. 7 directing one or more components of the sensing unit 110 of the status determination system 158 of FIG. 6 including to detect one or more spatial aspects of one or more portions of one or more of one or more of the objects 12 as first postural influencers of one or more of the subjects 10, which can be devices. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, the sensing unit 110 of the status determination system 158 can be used to detect spatial aspects, such as position, location, orientation, visual placement, visual appearance, and/or conformation of the objects 12.

For instance, in some implementations, the exemplary operation O21 may include the operation of O2110 for detecting one or more spatial aspects of one or more portions of one or more of the first postural influencers through at least in part one or more techniques involving one or more optical aspects. An exemplary implementation may include the optical detecting module 170j of FIG. 7 directing one or more of the optical based sensing components 110b of the status determination system 158 of FIG. 6 to detect one or more spatial aspects of one or more portions of one or more of the objects 12 as first postural influencers of one or more of the subjects 10, which can be devices, through at least in part one or more techniques involving one or more optical aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the optical based sensing components 110b of the status determination system 158 can be used to detect spatial aspects, such as position, location, orientation, visual placement, visual appearance, and/or conformation of the objects 12.

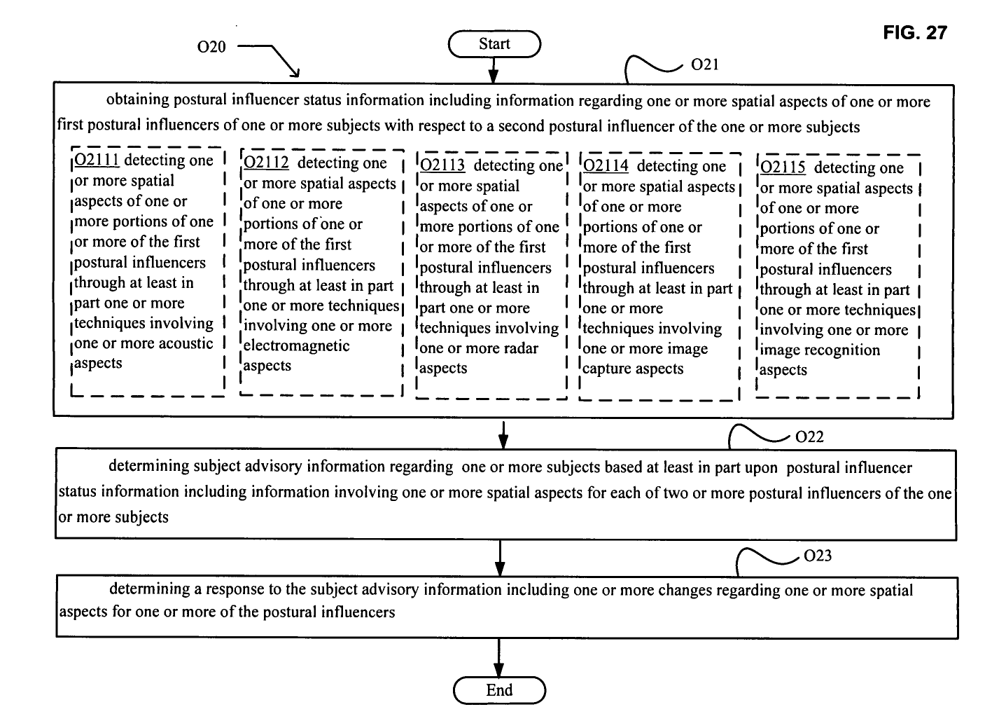

FIG. 27

FIG. 27 illustrates various implementations of the exemplary operation O21 of FIG. 24. In particular, FIG. 27 illustrates example implementations where the operation O21 includes one or more additional operations including, for example, operations O2111, O2112, O2113, O2114, and/or O2115, which may be executed generally by, in some instances, In particular, one or more sensing components of the sensing unit 110 of the status determination system 158 of FIG. 6.

For instance, in some implementations, the exemplary operation O21 may include the operation of O2111 for detecting one or more spatial aspects of one or more portions of one or more of the first postural influencers through at least in part one or more techniques involving one or more acoustic aspects. An exemplary implementation may include the acoustic detecting module 170k of FIG. 7 directing one or more of the acoustic based sensing components 110i of the sensing unit 110 of the status determination system 158 of FIG. 6 to detect one or more spatial aspects of one or more portions of one or more of the objects 12 as first postural influencers of one or more of the subjects 10, which can be devices, through at least in part one or more techniques involving one or more acoustic aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the acoustic based sensing components 110i of the status determination system 158 can be used to detect spatial aspects, such as position, location, orientation, visual placement, visual appearance, and/or conformation of the objects 12.

For instance, in some implementations, the exemplary operation O21 may include the operation of O2112 for detecting one or more spatial aspects of one or more portions of one or more of the first postural influencers through at least in part one or more techniques involving one or more electromagnetic aspects. An exemplary implementation may include the EM detecting module 170l of FIG. 7 directing one or more of the electromagnetic based sensing components 110g of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more electromagnetic aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the electromagnetic based sensing components 110g of the status determination system 158 can be used to detect spatial aspects, such as position, location, orientation, visual placement, visual appearance, and/or conformation of the objects 12.

For instance, in some implementations, the exemplary operation O21 may include the operation of O2113 for detecting one or more spatial aspects of one or more portions of one or more of the first postural influencers through at least in part one or more techniques involving one or more radar aspects. An exemplary implementation may include the radar detecting module 170m of FIG. 7 directing one or more of the radar based sensing components 110k of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more radar aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the radar based sensing components 110k of the status determination system 158 can be used to detect spatial aspects, such as position, location, orientation, visual placement, visual appearance, and/or conformation of the objects 12.

For instance, in some implementations, the exemplary operation O21 may include the operation of O2114 for detecting one or more spatial aspects of one or more portions of one or more of the first postural influencers through at least in part one or more techniques involving one or more image capture aspects. An exemplary implementation may include the image capture detecting module 170n of FIG. 7 directing one or more of the image capture based sensing components 110m of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more image capture aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the image capture based sensing components 110m of the status determination system 158 can be used to detect spatial aspects, such as position, location, orientation, visual placement, visual appearance, and/or conformation of the objects 12.

For instance, in some implementations, the exemplary operation O21 may include the operation of O2115 for detecting one or more spatial aspects of one or more portions of one or more of the first postural influencers through at least in part one or more techniques involving one or more image recognition aspects. An exemplary implementation may include the image recognition detecting module 170o of FIG. 7 directing one or more of the image recognition based sensing components 110l of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more image recognition aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the image recognition based sensing components 110l of the status determination system 158 can be used to detect spatial aspects, such as position, location, orientation, visual placement, visual appearance, and/or conformation of the objects 12.

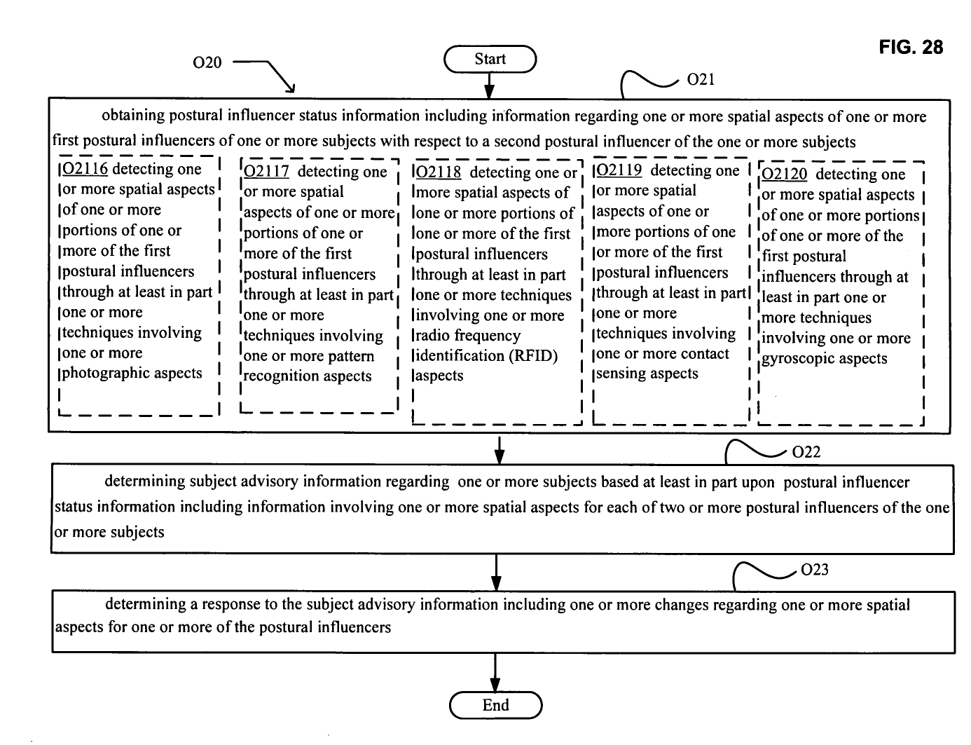

FIG. 28

FIG. 28 illustrates various implementations of the exemplary operation O21 of FIG. 24. In particular, FIG. 28 illustrates example implementations where the operation O21 includes one or more additional operations including, for example, operations O2116, O2117, O2118, O2119, and/or O2120, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O21 may include the operation of O2116 for detecting one or more spatial aspects of one or more portions of one or more of the first postural influencers through at least in part one or more techniques involving one or more photographic aspects. An exemplary implementation may include the photographic detecting module 170p of FIG. 7 directing one or more of the photographic based sensing components 110n of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more photographic aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the photographic based sensing components 110k of the status determination system 158 can be used to detect spatial aspects, such as position, location, orientation, visual placement, visual appearance, and/or conformation of the objects 12.

For instance, in some implementations, the exemplary operation O21 may include the operation of O2117 for detecting one or more spatial aspects of one or more portions of one or more of the first postural influencers through at least in part one or more techniques involving one or more pattern recognition aspects. An exemplary implementation may include the pattern recognition detecting module 170q of FIG. 7 directing one or more of the pattern recognition based sensing components 110e of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more pattern recognition aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the pattern recognition based sensing components 110k of the status determination system 158 can be used to detect spatial aspects, such as position, location, orientation, visual placement, visual appearance, and/or conformation of the objects 12.

For instance, in some implementations, the exemplary operation O21 may include the operation of O2118 for detecting one or more spatial aspects of one or more portions of one or more of the first postural influencers through at least in part one or more techniques involving one or more radio frequency identification (RFID) aspects. An exemplary implementation may include the RFID detecting module 170r of FIG. 7 directing one or more of the RFID based sensing components 110j of the sensing unit 110 of the status determination system 158 of FIG. 6 including to detect one or more spatial aspects of one or more portions of one or more of the objects 12 as first postural influencers of one or more of the subjects 10, which can be devices, through at least in part one or more techniques involving one or more RFID aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the RFID based sensing components 110k of the status determination system 158 can be used to detect spatial aspects, such as position, location, orientation, visual placement, visual appearance, and/or conformation of the objects 12.

For instance, in some implementations, the exemplary operation O21 may include the operation of O2119 for detecting one or more spatial aspects of one or more portions of one or more of the first postural influencers through at least in part one or more techniques involving one or more contact sensing aspects. An exemplary implementation may include the contact detecting module 170s of FIG. 7 directing one or more of the contact sensors 108l of one or more of the objects 12 as first postural influencers of one or more of the subjects 10 shown in FIG. 10 including to sense contact such as contact made with the objects by the subject 10, such as the subject touching a keyboard device as shown in FIG. 2 to detect one or more spatial aspects of one or more portions of the objects as postural influencers of one or more of the subjects 10. For instance, by sensing contact of the subject 10 (subject) of the object 12 (device), aspects of the orientation of the device with respect to the subject may be detected.

For instance, in some implementations, the exemplary operation O21 may include the operation of O2120 for detecting one or more spatial aspects of one or more portions of one or more of the first postural influencers through at least in part one or more techniques involving one or more gyroscopic aspects. An exemplary implementation may include the gyroscopic detecting module 170t of FIG. 7 directing one or more of the gyroscopic sensors 108f of one or more of the objects 12 as first postural influencers of one or more of the subjects 10 shown in FIG. 10 as postural influencers of one or more of the subjects 10. including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

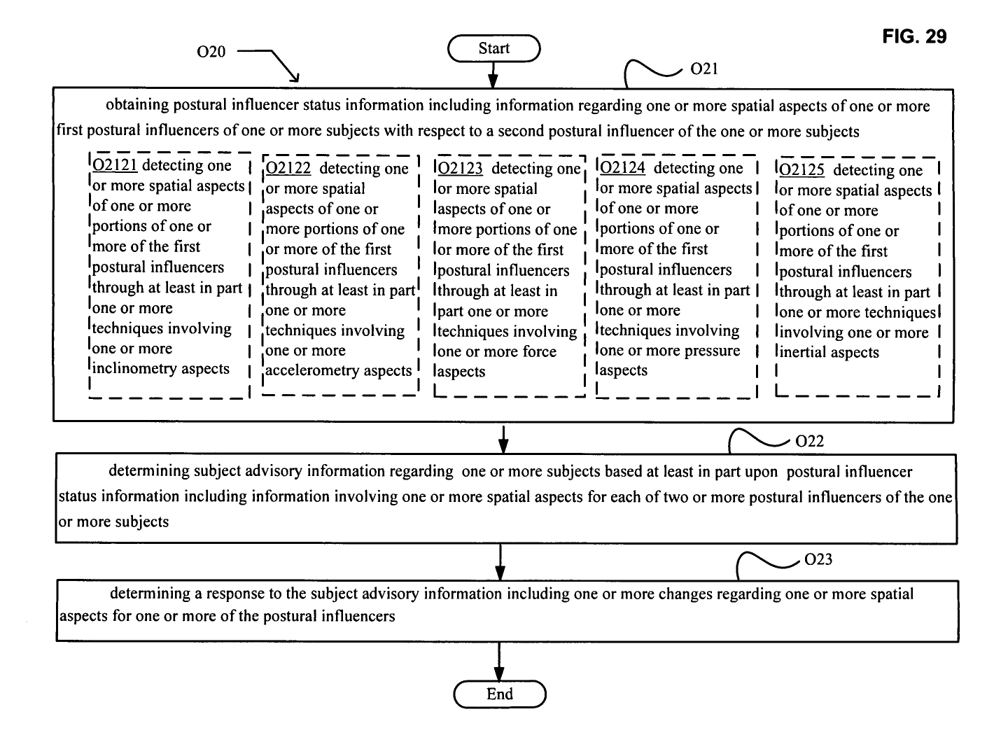

FIG. 29

FIG. 29 illustrates various implementations of the exemplary operation O21 of FIG. 24. In particular, FIG. 29 illustrates example implementations where the operation O21 includes one or more additional operations including, for example, operations O2121, O2122, O2123, O2124, and/or O2125, which may be executed generally by, in some instances, one or more of the sensors 108 of the object 12 of FIG. 10.

For instance, in some implementations, the exemplary operation O21 may include the operation of O2121 for detecting one or more spatial aspects of one or more portions of one or more of the first postural influencers through at least in part one or more techniques involving one or more inclinometry aspects. An exemplary implementation may include the inclinometry detecting module 170u of FIG. 7 directing one or more of the inclinometers 108i of one or more of the objects 12 as first postural influencers of one or more of the subjects 10 shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

For instance, in some implementations, the exemplary operation O21 may include the operation of O2122 for detecting one or more spatial aspects of one or more portions of one or more of the first postural influencers through at least in part one or more techniques involving one or more accelerometry aspects. An exemplary implementation may include the accelerometry detecting module 170v of FIG. 7 directing one or more of the accelerometers 108j of one or more of the objects 12 as first postural influencers of one or more of the subjects 10 shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

For instance, in some implementations, the exemplary operation O21 may include the operation of O2123 for detecting one or more spatial aspects of one or more portions of one or more of the first postural influencers through at least in part one or more techniques involving one or more force aspects. An exemplary implementation may include the force detecting module 170w of FIG. 7 directing one or more of the force sensors 108e of one or more of the objects 12 as first postural influencers of one or more of the subjects 10 shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

For instance, in some implementations, the exemplary operation O21 may include the operation of O2124 for detecting one or more spatial aspects of one or more portions of one or more of the first postural influencers through at least in part one or more techniques involving one or more pressure aspects. An exemplary implementation may include the pressure detecting module 170x of FIG. 7 directing one or more of the pressure sensors 108m of one or more of the objects 12 as first postural influencers of one or more of the subjects 10. shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

For instance, in some implementations, the exemplary operation O21 may include the operation of O2125 for detecting one or more spatial aspects of one or more portions of one or more of the first postural influencers through at least in part one or more techniques involving one or more inertial aspects. An exemplary implementation may include the inertial detecting module 170y of FIG. 7 directing one or more of the inertial sensors 108k of one or more of the objects 12 as first postural influencers of one or more of the subjects 10 shown in FIG. 10 including to detect one or more spatial aspects of the one or more portions of the device. Spatial aspects can include orientation visual placement, visual appearance, and/or conformation of the objects 12 involved and can be sent to the status determination system 158 as transmissions D1 and D2 by the objects as shown in FIG. 11.

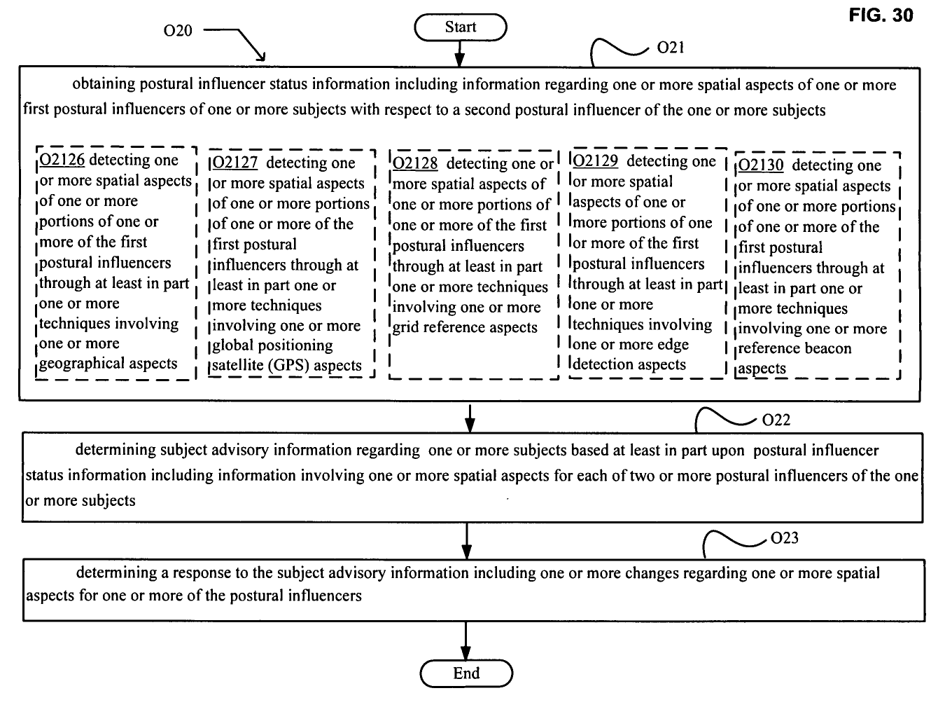

FIG. 30

FIG. 30 illustrates various implementations of the exemplary operation O21 of FIG. 24. In particular, FIG. 30 illustrates example implementations where the operation O21 includes one or more additional operations including, for example, operations O2126, O2127, O2128, O2129, and/or O2130, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O21 may include the operation of O2126 for detecting one or more spatial aspects of one or more portions of one or more of the first postural influencers through at least in part one or more techniques involving one or more geographical aspects. An exemplary implementation may include the geographical detecting module 170z of FIG. 7 directing one or more of the image recognition based sensing components 110l of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more geographical aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the image recognition based sensing components 110l of the status determination system 158 can be used to detect spatial aspects involving geographical aspects, such as position, location, orientation, visual placement, visual appearance, and/or conformation of the objects 12 in relation to a geographical landmark.

For instance, in some implementations, the exemplary operation O21 may include the operation of O2127 for detecting one or more spatial aspects of one or more portions of one or more of the first postural influencers through at least in part one or more techniques involving one or more global positioning satellite (GPS) aspects. An exemplary implementation may include the GPS detecting module 170aa of FIG. 7 directing one or more of the global positioning system (GPS) sensors 108g of one or more of the objects 12 as first postural influencers of one or more of the subjects 10 shown in FIG. 10 including to detect one or more spatial aspects of the one or more portions of the device. Spatial aspects can include location and position as provided by the global positioning system (GPS) to the global positioning system (GPS) sensors 108g of the objects 12 involved and can be sent to the status determination system 158 as transmissions D1 and D2 by the objects as shown in FIG. 11.

For instance, in some implementations, the exemplary operation O21 may include the operation of O2128 for detecting one or more spatial aspects of one or more portions of one or more of the first postural influencers through at least in part one or more techniques involving one or more grid reference aspects. An exemplary implementation may include the grid reference detecting module 170ab of FIG. 7 directing one or more of the grid reference based sensing components 110o of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more grid reference aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the grid reference based sensing components 110o of the status determination system 158 can be used to detect spatial aspects involving grid reference aspects, such as position, location, orientation, visual placement, visual appearance, and/or conformation of the objects 12.

For instance, in some implementations, the exemplary operation O21 may include the operation of O2129 for detecting one or more spatial aspects of one or more portions of one or more of the first postural influencers through at least in part one or more techniques involving one or more edge detection aspects. An exemplary implementation may include the edge detecting module 170ac of FIG. 7 directing one or more of the edge detection based sensing components 110p of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more edge detection aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the edge detection based sensing components 110p of the status determination system 158 can be used to detect spatial aspects involving edge detection aspects, such as position, location, orientation, visual placement, visual appearance, and/or conformation of the objects 12.

For instance, in some implementations, the exemplary operation O21 may include the operation of O2130 for detecting one or more spatial aspects of one or more portions of one or more of the first postural influencers through at least in part one or more techniques involving one or more reference beacon aspects. An exemplary implementation may include the beacon detecting module 170ad of FIG. 7 directing one or more of the reference beacon based sensing components 110q of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more reference beacon aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the reference beacon based sensing components 110q of the status determination system 158 can be used to detect spatial aspects involving reference beacon aspects, such as position, location, orientation, visual placement, visual appearance, and/or conformation of the objects 12.

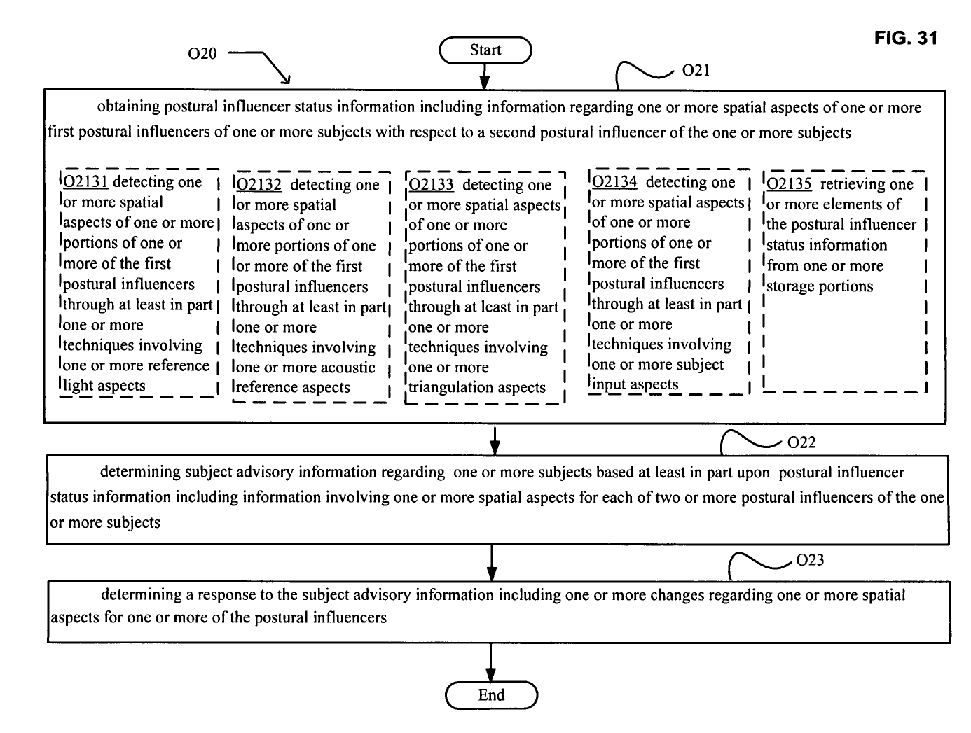

FIG. 31

FIG. 31 illustrates various implementations of the exemplary operation O21 of FIG. 24. In particular, FIG. 31 illustrates example implementations where the operation O21 includes one or more additional operations including, for example, operation O2131, O2132, O2133, O2134, and/or O2135, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O21 may include the operation of O2131 for detecting one or more spatial aspects of one or more portions of one or more of the first postural influencers through at least in part one or more techniques involving one or more reference light aspects. An exemplary implementation may include the reference light detecting module 170ae of FIG. 7 directing one or more of the reference light based sensing components 110r of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more reference light aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the reference light based sensing components 110r of the status determination system 158 can be used to detect spatial aspects involving reference light aspects, such as position, location, orientation, visual placement, visual appearance, and/or conformation of the objects 12.

For instance, in some implementations, the exemplary operation O21 may include the operation of O2132 for detecting one or more spatial aspects of one or more portions of one or more of the first postural influencers through at least in part one or more techniques involving one or more acoustic reference aspects. An exemplary implementation may include the acoustic reference detecting module 170af of FIG. 7 directing one or more of the acoustic reference based sensing components 110s of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more acoustic reference aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the acoustic reference based sensing components 110s of the status determination system 158 can be used to detect spatial aspects involving acoustic reference aspects, such as position, location, orientation, visual placement, visual appearance, and/or conformation of the objects 12.

For instance, in some implementations, the exemplary operation O21 may include the operation of O2133 for detecting one or more spatial aspects of one or more portions of one or more of the first postural influencers through at least in part one or more techniques involving one or more triangulation aspects. An exemplary implementation may include the triangulation detecting module 170ag of FIG. 7 directing one or more of the triangulation based sensing components 110t of the sensing unit 110 of the status determination system 158 of FIG. 6 including to detect one or more spatial aspects of one or more portions of one or more of the objects 12 as first postural influencers of one or more of the subjects 10 through at least in part one or more techniques involving one or more triangulation aspects. For example, in some implementations, the transmission D1 from object 1 carrying postural influencer status information regarding object 1 and the transmission D2 from object 2 carrying postural influencer status information about object 2 to the status determination system 158, as shown in FIG. 11, will not be present in situations in which the sensors 108 of the object 1 and object 2 are either not present or not being used. Consequently, in cases when the object sensors are not present or are otherwise not used, one or more of the triangulation based sensing components 110t of the status determination system 158 can be used to detect spatial aspects involving triangulation aspects, such as position, location, orientation, visual placement, visual appearance, and/or conformation of the objects 12.

For instance, in some implementations, the exemplary operation O21 may include the operation of O2134 for detecting one or more spatial aspects of one or more portions of one or more of the first postural influencers through at least in part one or more techniques involving one or more subject input aspects. An exemplary implementation may include the subject input module 170ah of FIG. 7 directing subject input aspects as detected by one or more of the contact sensors 108l of one or more of the objects 12 as first postural influencers of one or more of the subjects 10 shown in FIG. 10 including to sense contact such as contact made with the object by the subject 10, such as the subject touching a keyboard device as shown in FIG. 2 to detect one or more spatial aspects of one or more portions of the object as a device. For instance, by sensing contact by the subject 10 (subject) as subject input of the object 12 (device), aspects of the orientation of the object with respect to the subject may be detected.

For instance, in some implementations, the exemplary operation O21 may include the operation of O2135 for retrieving one or more elements of the postural influencer status information from one or more storage portions. An exemplary implementation may include the storage retrieving module 170aj of FIG. 8 directing the control unit 160 of the status determination unit 106 of the status determination system 158 of FIG. 6 including to retrieve one or more elements of postural influencer status information, such as dimensional aspects of one or more of the objects 12 as postural influencers of one or more of the subjects 10, from one or more storage portions, such as the storage unit 168, as part of obtaining postural influencer status information regarding one or more portions of the objects 12 (e.g. the object can be a device).

FIG. 31 illustrates various implementations of the exemplary operation O21 of FIG. 23. In particular, FIG. 31 illustrates example implementations where the operation O21 includes one or more additional operations including, for example, operation O2136, O2137, O2138, O2139, and/or O2140, which may be executed generally by, in some instances, one or more of the sensors 108 of the object 12 of FIG. 10 or one or more sensing components of the sensing unit 110 of the status determination system 158 of FIG. 6.

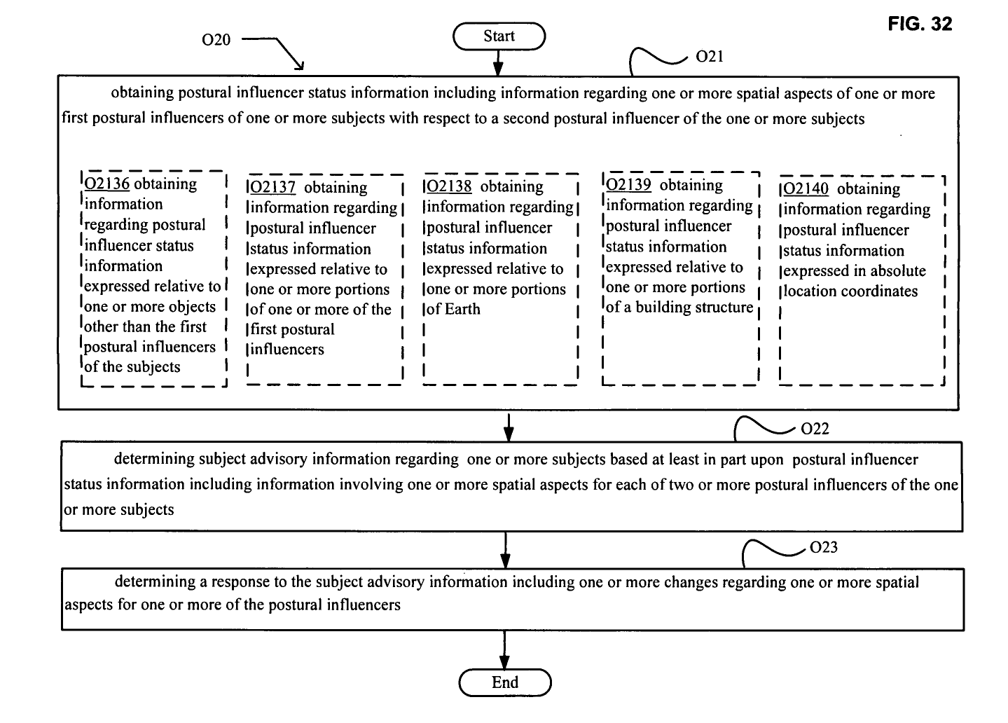

FIG. 32

FIG. 32 illustrates various implementations of the exemplary operation O21 of FIG. 24. In particular, FIG. 32 illustrates example implementations where the operation O21 includes one or more additional operations including, for example, operation O2136, O2137, O2138, O2139, and/or O2140, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O21 may include the operation of O2136 for obtaining information regarding postural influencer status information expressed relative to one or more objects other than the first postural influencers of the subjects. An exemplary implementation may include the object relative obtaining module 170ak of FIG. 8 directing one or more of the sensors 108 of the object 12 of FIG. 10 and/or one or more components of the sensing unit 110 of the status determination unit 158 including to obtain information regarding postural influencer status information expressed relative to one or more objects other than the objects 12 as first postural influencers of one or more of the subjects 10. For instance, in some implementations the obtained information can be related to positional or other spatial aspects of the objects 12 as related to one or more of the other objects 14 (such as structural members of a building, artwork, furniture, or other objects) that are not being used by the subject 10 or are otherwise not involved with influencing the subject regarding postural influencer status of the subject, such as posture. For instance, the spatial information obtained can be expressed in terms of distances between the objects 12 and the other objects 14.

For instance, in some implementations, the exemplary operation O21 may include the operation of O2137 for obtaining information regarding postural influencer status information expressed relative to one or more portions of one or more of the first postural influencers. An exemplary implementation may include the influencer relative module 170ay of FIG. 8 directing one or more of the sensors 108 of one or more of the objects 12 as postural influencers of one or more of the subjects 10 of FIG. 10 and/or one or more components of the sensing unit 110 of the status determination unit 158 to obtain information regarding postural influencer status information expressed relative to one or more of the objects 12 as first postural influencers. For instance, in some implementations the obtained information can be related to positional or other spatial aspects of the objects 12 as devices and the spatial information obtained about the objects can be expressed in terms of distances between the objects rather than expressed in terms of an absolute location for each of the objects.

For instance, in some implementations, the exemplary operation O21 may include the operation of O2138 for obtaining information regarding postural influencer status information expressed relative to one or more portions of Earth. An exemplary implementation may include the earth relative obtaining module 170am of FIG. 8 directing one or more of the sensors 108 of one or more of the objects 12 of FIG. 10 as first postural influencers of one or more of the subjects 10 and/or one or more components of the sensing unit 110 of the status determination unit 158 including to obtain information regarding postural influencer status information expressed relative to one or more of the objects 12 as postural influencers of one or more of the subjects 10. For instance, in some implementations the obtained information can be expressed relative to global positioning system (GPS) coordinates, geographical features or other aspects, or otherwise expressed relative to one or more portions of Earth.

For instance, in some implementations, the exemplary operation O21 may include the operation of O2139 for obtaining information regarding postural influencer status information expressed relative to one or more portions of a building structure. An exemplary implementation may include the building relative obtaining structure 170an of FIG. 8 directing one or more of the sensors 108 of one or more of the objects 12 of FIG. 10 as first postural influencers of one or more of the subjects 10 and/or one or more components of the sensing unit 110 of the status determination unit 158 including to obtain information regarding postural influencer status information expressed relative to one or more portions of a building structure. For instance, in some implementations the obtained information can be expressed relative to one or more portions of a building structure that houses the subject 10 and the objects 12 or is nearby to the subject and the objects.

For instance, in some implementations, the exemplary operation O21 may include the operation of O2140 for obtaining information regarding postural influencer status information expressed in absolute location coordinates. An exemplary implementation may include the locational obtaining module 170an of FIG. 8 directing one or more of the sensors 108 of one or more of the objects 12 of FIG. 10 as first postural influencers of one or more of the subjects 10 and/or one or more components of the sensing unit 110 of the status determination unit 158 including to obtain information regarding postural influencer status information expressed in absolute location coordinates. For instance, in some implementations the obtained information can be expressed in terms of global positioning system (GPS) coordinates.

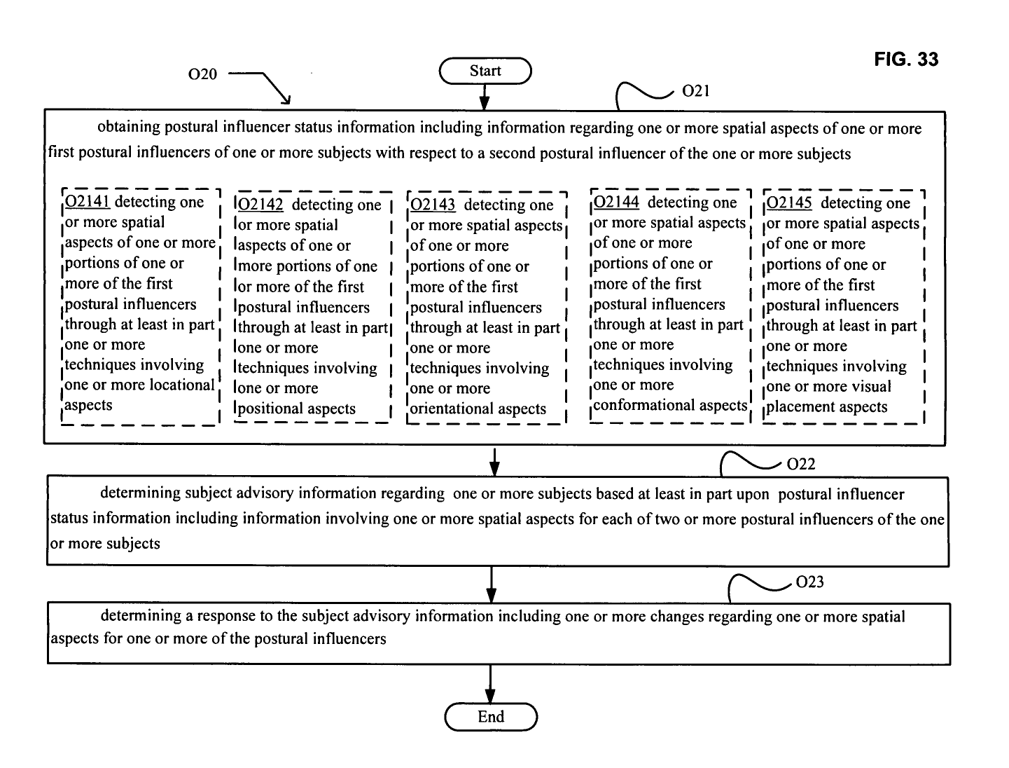

FIG. 33 illustrates various implementations of the exemplary operation O21 of FIG. 24. In particular, FIG. 33 illustrates example implementations where the operation O21 includes one or more additional operations including, for example, operation O2141, O2142, O2143, O2144, and/or O2145, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O21 may include the operation of O2141 for detecting one or more spatial aspects of one or more portions of one or more of the first postural influencers through at least in part one or more techniques involving one or more locational aspects. An exemplary implementation may include the locational detecting module 170ap of FIG. 8 directing one or more of the sensors 108 of one or more of the objects 12 of FIG. 10 as first postural influencers of one or more of the subjects 10 and/or one or more components of the sensing unit 110 of the status determination unit 158 including to detect one or more spatial aspects of one or more portions of one or more of the objects 12 as first postural influencers of one or more of the subjects 10 through at least in part one or more techniques involving one or more locational aspects. For instance, in some implementations the obtained information can be expressed in terms of global positioning system (GPS) coordinates or geographical coordinates.

For instance, in some implementations, the exemplary operation O21 may include the operation of O2142 for detecting one or more spatial aspects of one or more portions of one or more of the first postural influencers through at least in part one or more techniques involving one or more positional aspects. An exemplary implementation may include the positional detecting module 170aq of FIG. 8 directing one or more of the sensors 108 of one or more of the objects 12 of FIG. 10 as first postural influencers of one or more of the subjects 10 and/or one or more components of the sensing unit 110 of the status determination unit 158 including to detect one or more spatial aspects of one or more portions of one or more of the objects 12 as first postural influencers of one or more of the subjects 10 through at least in part one or more techniques involving one or more positional aspects. For instance, in some implementations the obtained information can be expressed in terms of global positioning system (GPS) coordinates or geographical coordinates.

For instance, in some implementations, the exemplary operation O21 may include the operation of O2143 for detecting one or more spatial aspects of one or more portions of one or more of the first postural influencers through at least in part one or more techniques involving one or more orientational aspects. An exemplary implementation may include the orientational detecting module 170ar of FIG. 8 directing one or more of the gyroscopic sensors 108f of one or more of the objects 12 as first postural influencers of one or more of the subjects 10 shown in FIG. 10 to detect one or more spatial aspects of the one or more portions of one or more of the objects as first postural influencers of one or more of the subjects 10. Spatial aspects can include orientation of the objects 12 involved and can be sent to the status determination system 158 as transmissions D1 and D2 by the objects as shown in FIG. 11.

For instance, in some implementations, the exemplary operation O21 may include the operation of O2144 for detecting one or more spatial aspects of one or more portions of one or more of the first postural influencers through at least in part one or more techniques involving one or more conformational aspects. An exemplary implementation may include the conformational detecting module 170 as of FIG. 8 directing one or more of the gyroscopic sensors 108f of one or more of the objects 12 as first postural influencers of one or more of the subjects 10 as a device shown in FIG. 10 including to detect one or more spatial aspects of the one or more portions of one or more of the objects as first postural influencers of one or more of the subjects 10. Spatial aspects can include conformation of the objects 12 involved and can be sent to the status determination system 158 as transmissions D1 and D2 by the objects as shown in FIG. 11.

For instance, in some implementations, the exemplary operation O21 may include the operation of O2145 for detecting one or more spatial aspects of one or more portions of one or more of the first postural influencers through at least in part one or more techniques involving one or more visual placement aspects. An exemplary implementation may include the visual placement module 170ay of FIG. 8 directing one or more of the display sensors 108n of one or more of the objects 12 as a device shown in FIG. 10, such as the object as a display device shown in FIG. 2, including to detect one or more spatial aspects of the one or more portions of one or more of the objects as first postural influencers of one or more of the subjects 10, such as placement of display features, such as icons, scene windows, scene widgets, window position, size of font, contrast, layering, etc, graphic or video content, or other visual features on the object 12 as a display device of FIG. 2.

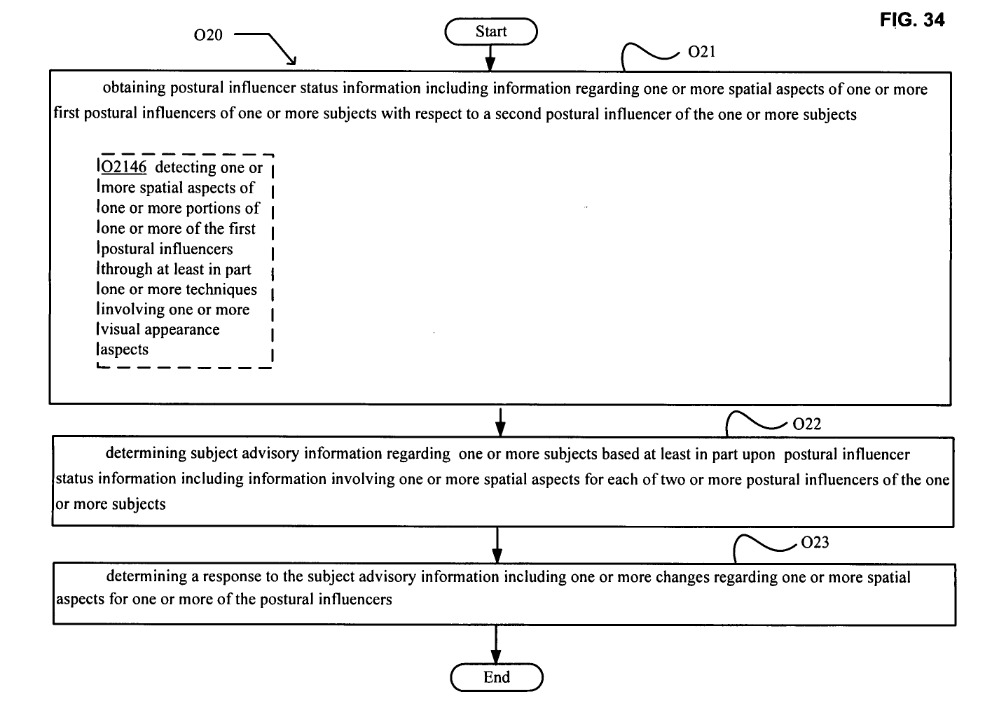

FIG. 34 illustrates various implementations of the exemplary operation O21 of FIG. 24. In particular, FIG. 34 illustrates example implementations where the operation O21 includes one or more additional operations including, for example, operation O2146, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O21 may include the operation of O2146 for detecting one or more spatial aspects of one or more portions of one or more of the first postural influencers through at least in part one or more techniques involving one or more visual appearance aspects. An exemplary implementation may include the visual appearance module 170aw of FIG. 8 directing one or more of the display sensors 108n of one or more of the objects 12 shown in FIG. 10 as first postural influencers of one or more of the subjects 10, such as the object as a display device shown in FIG. 2, including to detect one or more spatial aspects of the one or more portions of one or more of the objects as first postural influencers of one or more of the subjects 10, such as appearance, such as sizing, of display features, such as icons, scene windows, scene widgets, window position, size of font, contrast, layering, etc, graphic or video content, or other visual features on the object 12 as a display device of FIG. 2.

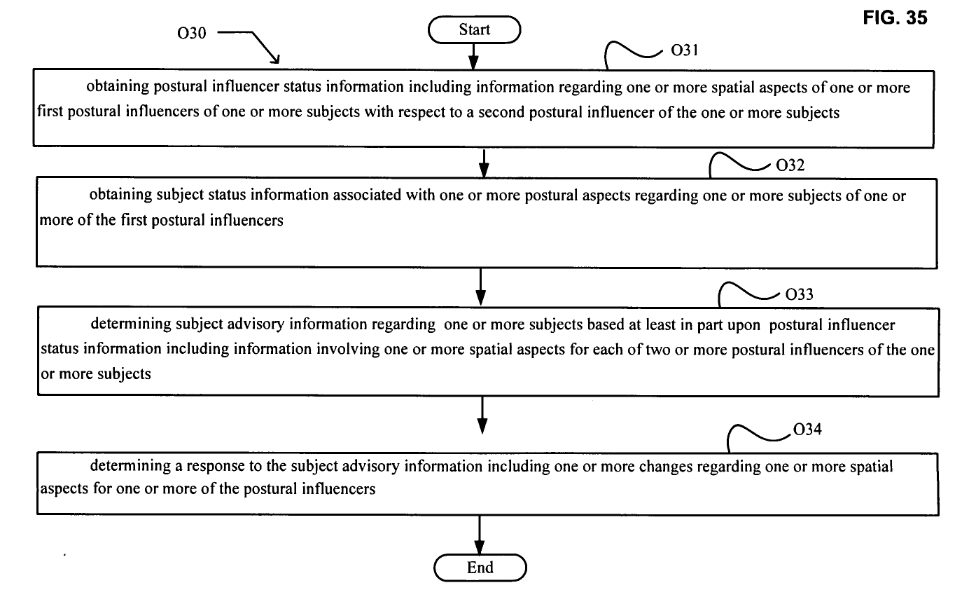

An operational flow O30 as shown in FIG. 35 represents example operations related to obtaining postural influencer status information, determining subject status information, and determining subject advisory information. In cases where the operational flows involve subjects and devices, as discussed above, in some implementations, the objects 12 can be devices and the subjects 10 can be subjects of the devices. FIG. 34 and those figures that follow may have various examples of operational flows, and explanation may be provided with respect to the above-described examples of FIGS. 1-14 and/or with respect to other examples and contexts. Nonetheless, it should be understood that the operational flows may be executed in a number of other environments and contexts, and/or in modified versions of FIGS. 1-14. Furthermore, although the various operational flows are presented in the sequence(s) illustrated, it should be understood that the various operations may be performed in other orders than those which are illustrated, or may be performed concurrently.

In FIG. 35 and those figures that follow, various operations may be depicted in a box-within-a-box manner. Such depictions may indicate that an operation in an internal box may comprise an optional exemplary implementation of the operational step illustrated in one or more external boxes. However, it should be understood that internal box operations may be viewed as independent operations separate from any associated external boxes and may be performed in any sequence with respect to all other illustrated operations, or may be performed concurrently.

After a start operation, the operational flow O30 may then move to operation O31, shown in FIG. 35, where obtaining postural influencer status information including information regarding one or more spatial aspects of one or more first postural influencers of one or more subjects with respect to a second postural influencer of the one or more subjects may be executed by, for example, the status determining system 158 of FIG. 6. An exemplary implementation may include the obtaining conformation module 170ax of FIG. 8 directing the status determination unit 106 of the status determination system 158 including to process postural influencer status information received by the communication unit 112 of the status determination system from one or more of the objects 12 as first postural influencers with respect to another object a second postural influencer and/or obtained through one or more of the components of the sensing unit 110 to determine subject status information. Subject status information could be determined through the use of components including the control unit 160 and the determination engine 167 of the status determining unit 106 indirectly based upon the postural influencer status information regarding the objects 12 such as the control unit 160 and the determination engine 167 may imply locational, positional, orientational and/or conformational information about one or more subjects based upon related information obtained or determined about the objects 12 involved. For instance, the subject 10 (human subject) of FIG. 2, may have certain locational, positional, orientational, or conformational status characteristics depending upon how the objects 12 (devices) of FIG. 2 are positioned relative to the subject. The subject 10 is depicted in FIG. 2 as viewing the object 12 (display device), which implies certain postural restriction for the subject and holding the object (probe device) to probe the procedure recipient, which implies other postural restriction. As depicted, the subject 10 of FIG. 2 has further requirements for touch and/or verbal interaction with one or more of the objects 12, which further imposes postural restriction for the subject. Various orientations or conformations of one or more of the objects 12 can impose even further postural restriction. Positional, locational, orientational, visual placement, visual appearance, and/or conformational information and possibly other postural influencer status information obtained about the objects 12 of FIG. 2 can be used by the control unit 160 and the determination engine 167 of the status determination unit 106 can imply a certain posture for the subject of FIG. 2 as an example of obtaining postural influencer status information including information regarding one or more spatial aspects of one or more first postural influencers of one or more subjects with respect to a second postural influencer of the one or more subjects. Other implementations of the status determination unit 106 can use postural influencer status information about the subject 10 obtained by the sensing unit 110 of the status determination system 158 of FIG. 6 alone or status of the objects 12 (as described immediately above) for obtaining postural influencer status information including information regarding one or more spatial aspects of one or more first postural influencers of one or more subjects with respect to a second postural influencer of the one or more subjects. For instance, in some implementations, postural influencer status information obtained by one or more components of the sensing unit 110, such as the radar based sensing component 110k, can be used by the status determination unit 106, such as for determining subject status information associated with positional, locational, orientation, and/or conformational information regarding the subject 10 and/or regarding the subject relative to the objects 12.

The operational flow O30 may move to an operation O32, shown in FIG. 35, where obtaining subject status information associated with one or more postural aspects regarding one or more subjects of one or more of the first postural influencers may be, executed by, for example, the obtaining information module 170ax of FIG. 8 directing the one of the sensing components of the sensing unit 110 of the status determination unit 158 of FIG. 6, such as the radar based sensing component 110k, in which, for example, in some implementations, the locations of the subjects 10 of FIG. 1 can be obtained by the radar based sensing component. In other implementations, other sensing components of the sensing unit 110 of FIG. 6 can be used to obtain subject status information associated with one or more postural aspects regarding the one or more subjects of two or more postural influencers, such as information regarding location, position, orientation, and/or conformation of the subjects. In other implementations, one or more of the sensors 108 of FIG. 10 found on one or more objects 12 assigned to monitor one or more of the subjects can be used in obtaining subject status information of the subjects, including information associated with one or more postural aspects regarding the one or more subjects. For example, in some implementations, the gyroscopic sensor 108f located on one or more of the objects 12 that are assigned to monitor one or more of the subjects 10 can be used for obtaining subject status information including information regarding orientational information of the subjects of other implementations, for example, the accelerometer 108j located on one or more of the objects 12 that are assigned to monitor one or more of the subjects 10 can be used in obtaining conformational information of the subjects such as how certain portions of each of the ore or more subjects are positioned relative to one another. For instance, the subject 10 of FIG. 2 entitled “human subject” is shown to have two out-stretched arms, a head in a cocked position, and legs spread apart to accommodate being subject of associated postural influencers such as the objects 12 shown.

To assist in obtaining the subject status information, for each of the subjects 10, the communication unit 112 of the one or more objects of FIG. 10 assigned to monitor the one or more subjects 10 can transmit the subject status information acquired by one or more of the sensors 108 to be received by the communication unit 112 of the status determination system 158 of FIG. 6.

The operational flow O30 may then move to operation O33, where determining subject advisory information regarding one or more subjects based at least in part upon postural influencer status information including information involving one or more spatial aspects for each of two or more postural influencers of the one or more subjects may be executed by, for example, the advisory resource unit 102 of the advisory system 118 of FIG. 3. An exemplary implementation may include, the determining advisory module 120q of FIG. 4 directing the advisory resource unit 102 including to receive the postural influencer status information from the status determination unit 106. As depicted in various Figures, the advisory resource unit 102 can be located in various entities including in a standalone version of the advisory system 118 (e.g. see FIG. 3) or in a version of the advisory system included in the object 12 (e.g. see FIG. 13) and the status determination unit can be located in various entities including the status determination system 158 (e.g. see FIG. 11) or in the objects 12 (e.g. see FIG. 14) so that some implementations include the status determination unit sending the postural influencer status information from the communication unit 112 of the status determination system 158 to the communication unit 112 of the advisory system and other implementations include the status determination unit sending the postural influencer status information to the advisory system internally within each of the objects. Once the postural influencer status information is received, the control unit 122 and the storage unit 130 (including in some implementations the guidelines 132) of the advisory resource unit 102 can determine subject advisory information. In some implementations, the subject advisory information is determined by the control unit 122 looking up various portions of the guidelines 132 contained in the storage unit 130 based upon the postural influencer status information. For example, the postural influencer status information may include locational or positional information for the objects 12 such as those objects depicted in FIG. 2. As an example, the control unit 122 may look up in the storage unit 130 portions of the guidelines associated with this information depicted in FIG. 2 to determine subject advisory information that would inform the subject 10 of FIG. 2 that the subject has been in a posture that over time could compromise integrity of a portion of the subject, such as the trapezius muscle or one or more vertebrae of the subject's spinal column. The subject advisory information could further include one or more suggestions regarding modifications to the existing posture of the subject 10 that may be implemented by repositioning one or more of the objects 12 so that the subject 10 can still use or otherwise interact with the objects in a more desired posture thereby alleviating potential ill effects by substituting the present posture of the subject with a more desired posture. In other implementations, the control unit 122 of the advisory resource unit 102 can include generation of subject advisory information through input of the subject status information into a physiological-based simulation model contained in the memory unit 128 of the control unit, which may then advise of suggested changes to the subject status, such as changes in posture. The control unit 122 of the advisory resource unit 102 may then determine suggested modifications to the physical status of the objects 12 (devices) based upon the postural influencer status information for the objects that was received. These suggested modifications can be incorporated into the determined subject advisory information.

The operational flow O30 may then move to operation O34, where determining a response to the subject advisory information including one or more changes regarding one or more spatial aspects for one or more of the postural influencers may be executed by, for example, the status determining system 158 of FIG. 6.

An exemplary implementation may include after the subject advisory information is determined, the response determining module ah directing the status determination unit 106 of the status determination system 158 to process postural influencer status information received by the communication unit 112 of the status determination system from one or more of the objects 12 as postural influencers or other of the postural influencers 13 with respect to another postural influencer and/or obtained through one or more of the components of the sensing unit 110 regarding one or more spatial aspects for one or more of the postural influencers. The postural influencer status information is processed by the status determination unit 106 to ascertain a response to the determined status advisory information such as changes in one or more spatial aspects of one or more of the postural influencers as objects 12 and/or other postural influencers 13. For instance, spatial aspects can include location, position, orientation, conformational and/or other spatial aspects relative to other of the objects 12 or other of the postural influencers 13 or relative to other references such as particular portions of a room or other environment of the objects or other postural influencers. Postural influencer status information could be determined through the use of components including the control unit 160 and the determination engine 167 of the status determining unit 106.

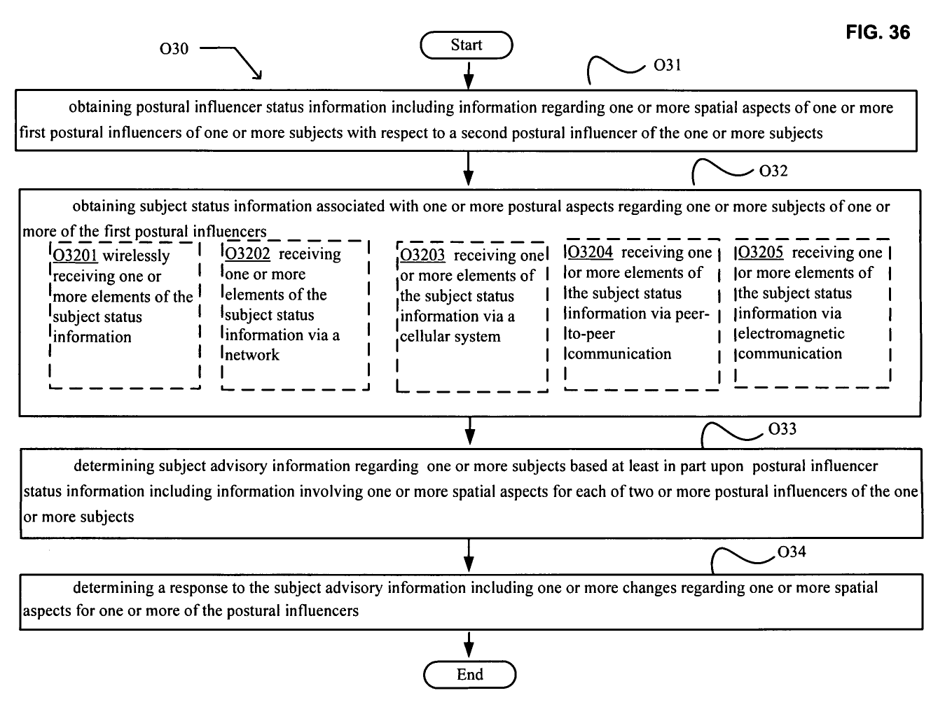

FIG. 36

FIG. 36 illustrates various implementations of the exemplary operation O32 of FIG. 35. In particular, FIG. 36 illustrates example implementations where the operation O32 includes one or more additional operations including, for example, operations O3201, O3202, O3203, O3204, and/or O3205, which may be executed generally by, in some instances, one or more of the transceiver components 156 of the communication unit 112 of the status determining system 158 of FIG. 6.

For instance, in some implementations, the exemplary operation O32 may include the operation of O3201 for wirelessly receiving one or more elements of the subject status information. An exemplary implementation may include the wireless receiving module 170a of FIG. 7 directing one or more of the wireless transceiver components 156b of the communication unit 112 of the status determination system 158 of FIG. 6 including to receive wireless transmissions from each wireless transceiver component 156b of FIG. 10 of the communication unit 112 of one or more of the objects 12 assigned to monitor one or more of the subjects 10. For example, in some implementations, the transmission D1 from object 1 carrying subject status information regarding the subject 10 and the transmission D2 from object 2 carrying subject status information about the subject to the status determination system 158, as shown in FIG. 11, can be sent and received by the wireless transceiver components 156b of the objects 12 and the status determination system 158, respectively, as wireless transmissions.

For instance, in some implementations, the exemplary operation O32 may include the operation of O3202 for receiving one or more elements of the subject status information via a network. An exemplary implementation may include the network receiving module 170b of FIG. 7 directing one or more of the network transceiver components 156a of the communication unit 112 of the status determination system 158 of FIG. 6 including to receive network transmissions from each network transceiver component 156a of FIG. 10 of the communication unit 112 of the objects 12 assigned to monitor one or more of the subjects 10. For example, in some implementations, the transmission D1 from object 1 carrying subject status information regarding the subject 10 and the transmission D2 from object 2 carrying subject status information about the subject to the status determination system 158, as shown in FIG. 11, can be sent and received by the network transceiver components 156a of the objects 12 and the status determination system 158, respectively, as network transmissions.

For instance, in some implementations, the exemplary operation O32 may include the operation of O3203 for receiving one or more elements of the subject status information via a cellular system. An exemplary implementation may include the cellular receiving module 170c of FIG. 7 directing one or more of the cellular transceiver components 156c of the communication unit 112 of the status determination system 158 of FIG. 6 including to receive cellular transmissions from each cellular transceiver component 156a of FIG. 10 of the communication unit 112 of one or more of the objects 12 assigned to monitor one or more of the subjects 10. For example, in some implementations, the transmission D1 from object 1 carrying subject status information regarding object 1 and the transmission D2 from object 2 carrying subject status information about object 2 to the status determination system 158, as shown in FIG. 11, can be sent and received by the cellular transceiver components 156c of the objects 12 and the status determination system 158, respectively, as cellular transmissions.

For instance, in some implementations, the exemplary operation O32 may include the operation of O3204 for receiving one or more elements of the subject status information via peer-to-peer communication. An exemplary implementation may include the peer-to-peer receiving module 170d of FIG. 7 directing one or more of the peer-to-peer transceiver components 156d of the communication unit 112 of the status determination system 158 of FIG. 6 including to receive peer-to-peer transmissions from each peer-to-peer transceiver component 156d of FIG. 10 of the communication unit 112 of one or more of the objects 12 assigned to monitor one or more of the subjects 10. For example, in some implementations, the transmission D1 from object 1 carrying subject status information regarding the subject 10 and the transmission D2 from object 2 carrying subject status information about the subject 10 to the status determination system 158, as shown in FIG. 11, can be sent and received by the peer-to-peer transceiver components 156d of the objects 12 and the status determination system 158, respectively, as peer-to-peer transmissions.

For instance, in some implementations, the exemplary operation O32 may include the operation of O3205 for receiving one or more elements of the subject status information via electromagnetic communication. An exemplary implementation may include the EM receiving module 170e of FIG. 7 directing one or more of the electromagnetic communication transceiver components 156e of the communication unit 112 of the status determination system 158 of FIG. 6 including to receive electromagnetic communication transmissions from each electromagnetic communication transceiver component 156a of FIG. 10 of the communication unit 112 of one or more of the objects 12 assigned to monitor one or more of the subjects 10. For example, in some implementations, the transmission D1 from object 1 carrying subject status information regarding the subject 10 and the transmission D2 from object 2 carrying subject status information about the subject to the status determination system 158, as shown in FIG. 11, can be sent and received by the electromagnetic communication transceiver components 156c of the objects 12 and the status determination system 158, respectively, as electromagnetic communication transmissions.

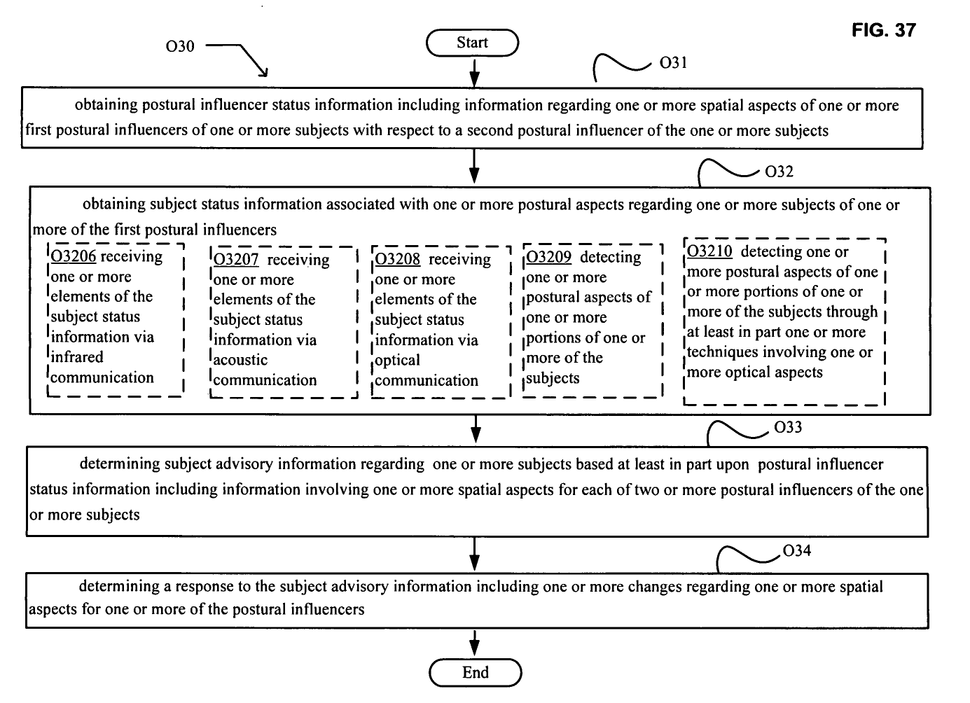

FIG. 37

FIG. 37 illustrates various implementations of the exemplary operation O32 of FIG. 35. In particular, FIG. 37 illustrates example implementations where the operation O32 includes one or more additional operations including, for example, operations O3206, O3207, O3208, O3209, and/or O3210, which may be executed generally by, in some instances, one or more of the transceiver components 156 of the communication unit 112 or one or more sensing components of the sensing unit 110 of the status determination system 158 of FIG. 6.

For instance, in some implementations, the exemplary operation O32 may include the operation of O3206 for receiving one or more elements of the subject status information via infrared communication. An exemplary implementation may include the infrared receiving module 170f of FIG. 7 directing one or more of the infrared transceiver components 156f of the communication unit 112 of the status determination system 158 of FIG. 6 including to receive infrared transmissions from each infrared transceiver component 156f of FIG. 10 of the communication unit 112 one or more of the objects 12 assigned to monitor one or more of the subjects 10. For example, in some implementations, the transmission D1 from object 1 carrying subject status information regarding the subject 10 and the transmission D2 from object 2 carrying subject status information about the subject to the status determination system 158, as shown in FIG. 11, can be sent and received by the infrared transceiver components 156c of the objects 12 and the status determination system 158, respectively, as infrared transmissions.

For instance, in some implementations, the exemplary operation O32 may include the operation of O3207 for receiving one or more elements of the subject status information via acoustic communication. the acoustic receiving module 170g of FIG. 7 directing one or more of the acoustic transceiver components 156g of the communication unit 112 of the status determination system 158 of FIG. 6 including to receive acoustic transmissions from each acoustic transceiver component 156g of FIG. 10 of the communication unit 112 one or more of the objects 12 assigned to monitor one or more of the subjects 10. For example, in some implementations, the transmission D1 from object 1 carrying subject status information regarding object 1 and the transmission D2 from object 2 carrying subject status information about the subject 10 to the status determination system 158, as shown in FIG. 11, can be sent and received by the acoustic transceiver components 156g of the objects 12 and the status determination system 158, respectively, as acoustic transmissions.

For instance, in some implementations, the exemplary operation O32 may include the operation of O3208 for receiving one or more elements of the subject status information via optical communication. An exemplary implementation may include the optical receiving module 170h of FIG. 7 directing one or more of the optical transceiver components 156h of the communication unit 112 of the status determination system 158 of FIG. 6 including to receive optical transmissions from each optical transceiver component 156h of FIG. 10 of the communication unit 112 of one or more of the objects 12 assigned to monitor one or more of the subjects 10. For example, in some implementations, the transmission D1 from object 1 carrying subject status information regarding the subject 10 and the transmission D2 from object 2 carrying subject status information about the subject 10 to the status determination system 158, as shown in FIG. 11, can be sent and received by the optical transceiver components 156h of the objects 12 and the status determination system 158, respectively, as optical transmissions.

For instance, in some implementations, the exemplary operation O32 may include the operation of O3209 for detecting one or more postural aspects of one or more portions of one or more of the subjects. An exemplary implementation can include the detecting module 170i of FIG. 7 directing one or more components of the sensing unit 110 of the status determination system 158 of FIG. 6 including to detect one or more postural aspects of one or more portions of one or more of the subjects 10. For example, in some implementations, the transmission D1 from object 1 carrying subject status information regarding the subject 10 and the transmission D2 from object 2 carrying subject status information about the subject 10 to the status determination system 158, as shown in FIG. 11, will not be present in situations in which the sensors 108 of the object 1 and object 2 are either not present or not being used. Consequently, in cases when the object sensors are not present or are otherwise not used, the sensing unit 110 of the status determination system 158 can be used to detect postural aspects, such as position, location, orientation, and/or conformation of one or more of the subjects 10.

For instance, in some implementations, the exemplary operation O32 may include the operation of O3210 for detecting one or more postural aspects of one or more portions of one or more of the subjects through at least in part one or more techniques involving one or more optical aspects. An exemplary implementation may include the optical detecting module 170j of FIG. 7 directing one or more of the optical based sensing components 110b of the sensing unit 110 of the status determination system 158 of FIG. 6 including to detect one or more postural aspects of one or more portions of one or more of the subjects 10 through at least in part one or more techniques involving one or more optical aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the optical based sensing components 110b of the status determination system 158 can be used to detect postural aspects, such as position, location, orientation, and/or conformation of the objects 12.

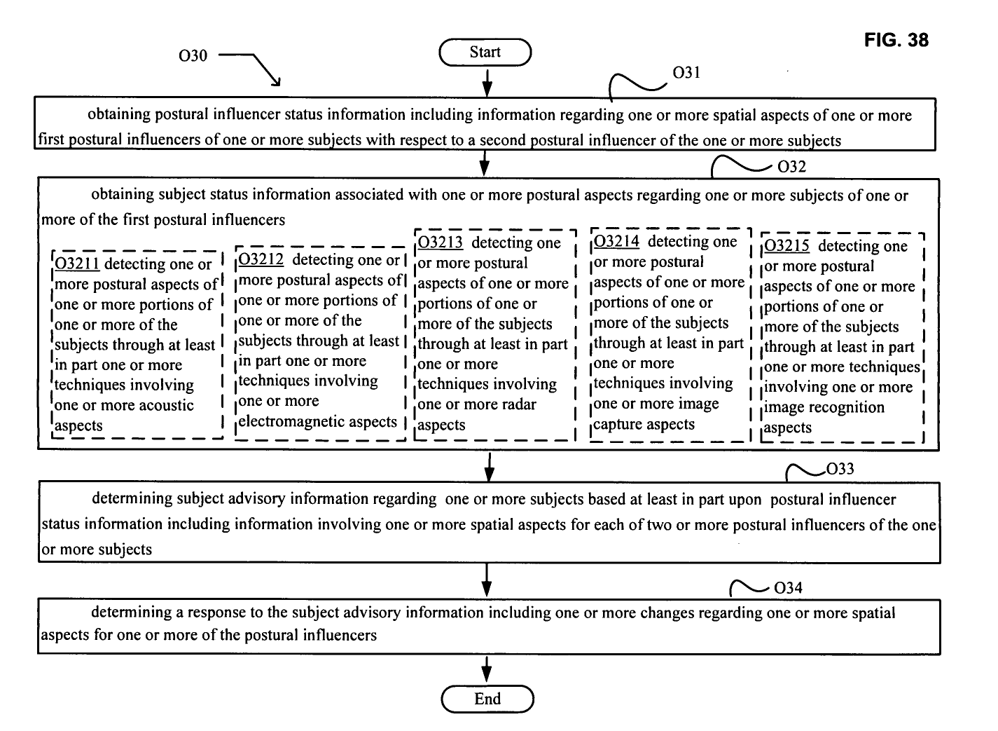

FIG. 38

FIG. 38 illustrates various implementations of the exemplary operation O32 of FIG. 35. In particular, FIG. 38 illustrates example implementations where the operation O32 includes one or more additional operations including, for example, operations O3211, O3212, O3213, O3214, and/or O3215, which may be executed generally by, in some instances, In particular, one or more sensing components of the sensing unit 110 of the status determination system 158 of FIG. 6.

For instance, in some implementations, the exemplary operation O32 may include the operation of O3211 for detecting one or more postural aspects of one or more portions of one or more of the subjects through at least in part one or more techniques involving one or more acoustic aspects. An exemplary implementation may include the acoustic detecting module 170k of FIG. 7 directing one or more of the acoustic based sensing components 110i of the sensing unit 110 of the status determination system 158 of FIG. 6 including to detect one or more postural aspects of one or more portions of one or more of the subjects 10 through at least in part one or more techniques involving one or more acoustic aspects. For example, in some implementations, the transmission D1 from object 1 carrying subject status information regarding the subject 10 and the transmission D2 from object 2 carrying subject status information about the subject 10 to the status determination system 158, as shown in FIG. 11, will not be present in situations in which the sensors 108 of the object 1 and object 2 are either not present or not being used. Consequently, in cases when the object sensors are not present or are otherwise not used, one or more of the acoustic based sensing components 110i of the status determination system 158 can be used to detect spatial aspects, such as position, location, orientation, and/or conformation of one or more of the subjects 10.

For instance, in some implementations, the exemplary operation O32 may include the operation of O3212 for detecting one or more postural aspects of one or more portions of one or more of the subjects through at least in part one or more techniques involving one or more electromagnetic aspects. An exemplary implementation may include the EM detecting module 170l of FIG. 7 directing one or more of the electromagnetic based sensing components 110g of the sensing unit 110 of the status determination system 158 of FIG. 6 including to detect one or more postural aspects of one or more portions of one or more of the subjects 10 through at least in part one or more techniques involving one or more electromagnetic aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the electromagnetic based sensing components 110g of the status determination system 158 can be used to detect postural aspects, such as position, location, orientation, visual placement, visual appearance, and/or conformation of the one or more of the subjects 10.

For instance, in some implementations, the exemplary operation O32 may include the operation of O3213 for detecting one or more postural aspects of one or more portions of one or more of the subjects through at least in part one or more techniques involving one or more radar aspects. An exemplary implementation may include the radar detecting module 170m of FIG. 7 directing one or more of the radar based sensing components 110k of the sensing unit 110 of the status determination system 158 of FIG. 6 including to detect one or more postural aspects of one or more portions of one or more of the subjects 10 through at least in part one or more techniques involving one or more radar aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the radar based sensing components 110k of the status determination system 158 can be used to detect postural aspects, such as position, location, orientation, and/or conformation of the one or more of the subjects 10.

For instance, in some implementations, the exemplary operation O32 may include the operation of O3214 for detecting one or more postural aspects of one or more portions of one or more of the subjects through at least in part one or more techniques involving one or more image capture aspects. An exemplary implementation may include the image capture detecting module 170n of FIG. 7 directing one or more of the image capture based sensing components 110m of the sensing unit 110 of the status determination system 158 of FIG. 6 including to detect one or more postural aspects of one or more portions of one or more of the subjects 10 through at least in part one or more techniques involving one or more image capture aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the image capture based sensing components 110m of the status determination system 158 can be used to detect postural aspects, such as position, location, orientation, and/or conformation of one or more of the subjects 10.

For instance, in some implementations, the exemplary operation O32 may include the operation of O3215 for detecting one or more postural aspects of one or more portions of one or more of the subjects through at least in part one or more techniques involving one or more image recognition aspects. An exemplary implementation may include the image recognition detecting module 170o of FIG. 7 directing one or more of the image recognition based sensing components 110l of the sensing unit 110 of the status determination system 158 of FIG. 6 including to detect one or more postural aspects of one or more portions of one or more of the subjects 10 through at least in part one or more techniques involving one or more image recognition aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the image recognition based sensing components 110l of the status determination system 158 can be used to detect postural aspects, such as position, location, orientation, and/or conformation of one or more of the subjects 10.

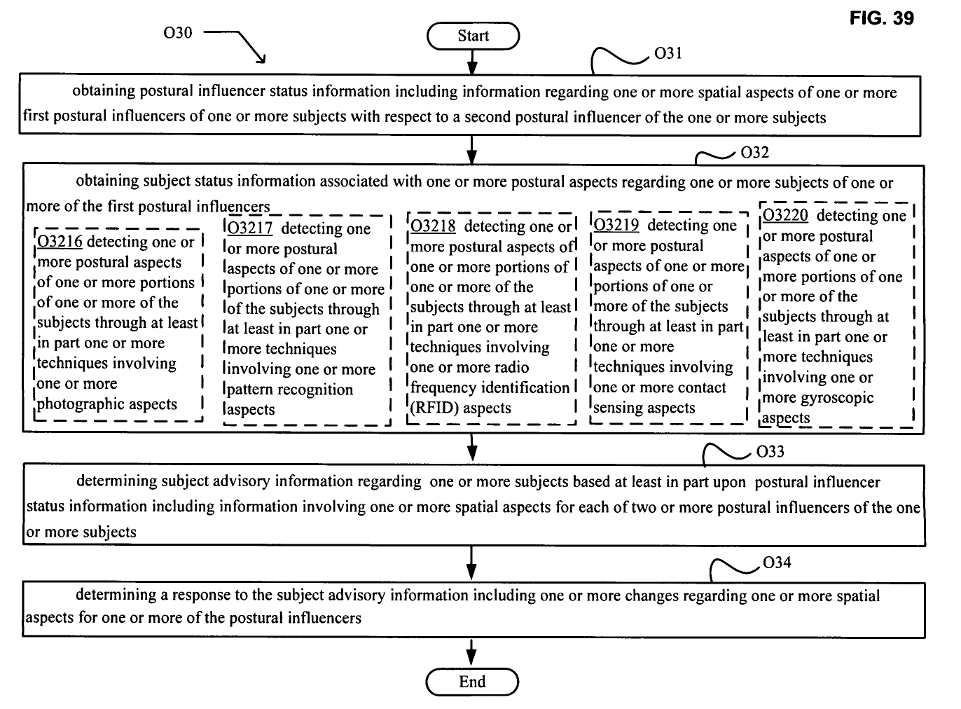

FIG. 39

FIG. 39 illustrates various implementations of the exemplary operation O32 of FIG. 35. In particular, FIG. 39 illustrates example implementations where the operation O32 includes one or more additional operations including, for example, operations O3216, O3217, O3218, O3219, and/or O3220, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O32 may include the operation of O3216 for detecting one or more postural aspects of one or more portions of one or more of the subjects through at least in part one or more techniques involving one or more photographic aspects. An exemplary implementation may include the photographic detecting module 170p of FIG. 7 directing one or more of the photographic based sensing components 110n of the sensing unit 110 of the status determination system 158 of FIG. 6 including to detect one or more postural aspects of one or more portions of one or more of the subjects 10 through at least in part one or more techniques involving one or more photographic aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the photographic based sensing components 110k of the status determination system 158 can be used to detect postural aspects, such as position, location, orientation, and/or conformation of one or more of the subjects 10.

For instance, in some implementations, the exemplary operation O32 may include the operation of O3217 for detecting one or more postural aspects of one or more portions of one or more of the subjects through at least in part one or more techniques involving one or more pattern recognition aspects. An exemplary implementation may include the pattern recognition detecting module 170q of FIG. 7 directing one or more of the pattern recognition based sensing components 110e of the sensing unit 110 of the status determination system 158 of FIG. 6 including to detect one or more postural aspects of one or more portions of one or more of the subjects 10 through at least in part one or more techniques involving one or more pattern recognition aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the pattern recognition based sensing components 110k of the status determination system 158 can be used to detect postural aspects, such as position, location, orientation, and/or conformation of one or more of the subjects 10.

For instance, in some implementations, the exemplary operation O32 may include the operation of O3218 for detecting one or more postural aspects of one or more portions of one or more of the subjects through at least in part one or more techniques involving one or more radio frequency identification (RFID) aspects. An exemplary implementation may include the RFID detecting module 170r of FIG. 7 directing one or more of the RFID based sensing components 110j of the sensing unit 110 of the status determination system 158 of FIG. 6 including to detect one or more postural aspects of one or more portions of one or more of the subjects 10 through at least in part one or more techniques involving one or more RFID aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the RFID based sensing components 110k of the status determination system 158 can be used to detect postural aspects, such as position, location, orientation, and/or conformation of one or more of the subjects 10.

For instance, in some implementations, the exemplary operation O32 may include the operation of O3219 for detecting one or more postural aspects of one or more portions of one or more of the subjects through at least in part one or more techniques involving one or more contact sensing aspects. An exemplary implementation may include the contact detecting module 170s of FIG. 7 directing one or more of the contact sensors 108l of one or more of the objects 12 shown in FIG. 10 assigned to monitor one or more of the subjects including to sense contact such as contact made by the subject 10, such as the subject touching another one of the objects such as a keyboard device as shown in FIG. 2 to detect one or more postural aspects of one or more portions of the subject. For instance, by sensing contact by the subject 10 (subject) with another one of the object 12 (device), postural aspects, such as orientation, of the subject with respect to the object may be detected.

For instance, in some implementations, the exemplary operation O32 may include the operation of O3220 for detecting one or more postural aspects of one or more portions of one or more of the subjects through at least in part one or more techniques involving one or more gyroscopic aspects. An exemplary implementation may include the gyroscopic detecting module 170t of FIG. 7 directing one or more of the gyroscopic sensors 108f of one or more of the objects 12 shown in FIG. 10 assigned to monitor one or more of the subjects including to detect one or more postural aspects of the one or more portions of the subject. Postural aspects can include orientation, and/or conformation of the one or more subjects 12 involved and can be sent to the status determination system 158 as transmissions D1 and D2 by the objects as shown in FIG. 11.

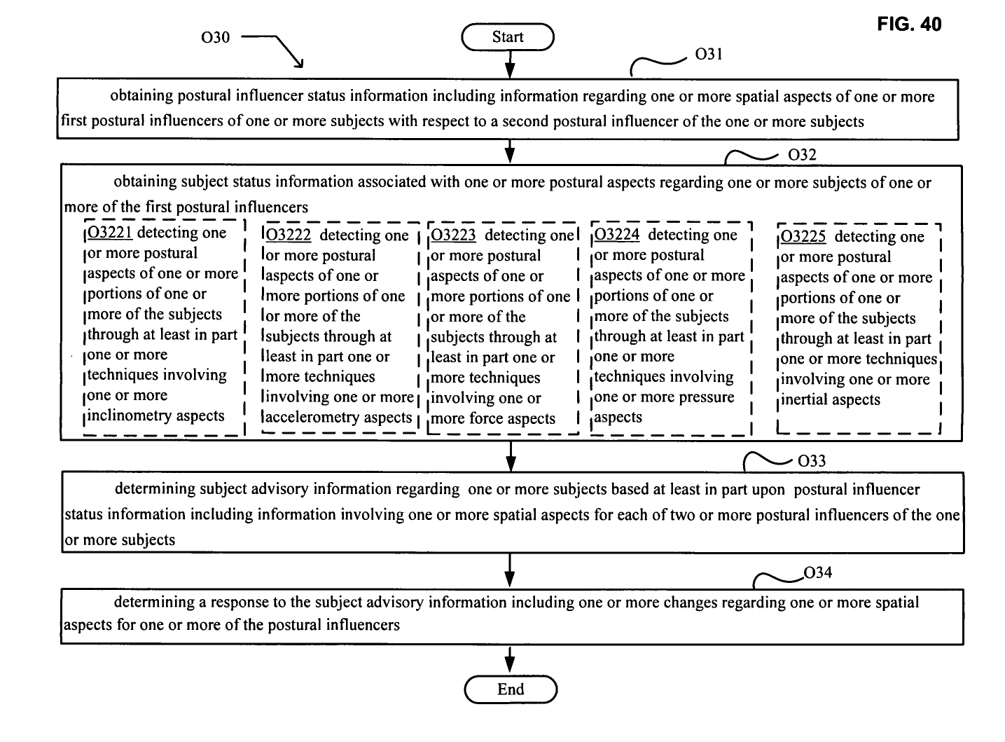

FIG. 40

FIG. 40 illustrates various implementations of the exemplary operation O32 of FIG. 35. In particular, FIG. 40 illustrates example implementations where the operation O32 includes one or more additional operations including, for example, operations O3221, O3222, O3223, O3224, and/or O3225, which may be executed generally by, in some instances, one or more of the sensors 108 of the object 12 of FIG. 10.

For instance, in some implementations, the exemplary operation O32 may include the operation of O3221 for detecting one or more postural aspects of one or more portions of one or more of the subjects through at least in part one or more techniques involving one or more inclinometry aspects. An exemplary implementation may include the inclinometry detecting module 170u of FIG. 7 directing one or more of the inclinometers 108i of one or more of the objects 12 shown in FIG. 10 assigned to monitor one or more of the subjects including to detect one or more postural aspects of the one or more portions of the subject. Postural aspects can include orientation, and/or conformation of the one or more subjects 12 involved and can be sent to the status determination system 158 as transmissions D1 and D2 by the objects as shown in FIG. 11.

For instance, in some implementations, the exemplary operation O32 may include the operation of O3222 for detecting one or more postural aspects of one or more portions of one or more of the subjects through at least in part one or more techniques involving one or more accelerometry aspects. An exemplary implementation may include the accelerometry detecting module 170v of FIG. 7 directing one or more of the accelerometers 108j of one or more of the objects 12 shown in FIG. 10 assigned to monitor one or more of the subjects including to detect one or more postural aspects of the one or more portions of the subject. Postural aspects can include orientation, and/or conformation of the one or more subjects 12 involved and can be sent to the status determination system 158 as transmissions D1 and D2 by the objects as shown in FIG. 11.

For instance, in some implementations, the exemplary operation O32 may include the operation of O3223 for detecting one or more postural aspects of one or more portions of one or more of the subjects through at least in part one or more techniques involving one or more force aspects. An exemplary implementation may include the force detecting module 170w of FIG. 7 directing one or more of the force sensors 108e of one or more of the objects 12 shown in FIG. 10 assigned to monitor one or more of the subjects including to detect one or more postural aspects of the one or more portions of the subject. Postural aspects can include orientation, and/or conformation of the one or more subjects 12 involved and can be sent to the status determination system 158 as transmissions D1 and D2 by the objects as shown in FIG. 11.

For instance, in some implementations, the exemplary operation O32 may include the operation of O3224 for detecting one or more postural aspects of one or more portions of one or more of the subjects through at least in part one or more techniques involving one or more pressure aspects. An exemplary implementation may include the pressure detecting module 170x of FIG. 7 directing one or more of the pressure sensors 108m of one or more of the objects 12 shown in FIG. 10 assigned to monitor one or more of the subjects including to detect one or more postural aspects of the one or more portions of the subject. Postural aspects can include orientation, and/or conformation of the one or more subjects 12 involved and can be sent to the status determination system 158 as transmissions D1 and D2 by the objects as shown in FIG. 11.

For instance, in some implementations, the exemplary operation O32 may include the operation of O3225 for detecting one or more postural aspects of one or more portions of one or more of the subjects through at least in part one or more techniques involving one or more inertial aspects. An exemplary implementation may include the inertial detecting module 170y of FIG. 7 directing one or more of the inertial sensors 108k of one or more of the objects 12 shown in FIG. 10 assigned to monitor one or more of the subjects including to detect one or more postural aspects of the one or more portions of the subject. Postural aspects can include orientation, and/or conformation of the one or more subjects 12 involved and can be sent to the status determination system 158 as transmissions D1 and D2 by the objects as shown in FIG. 11.

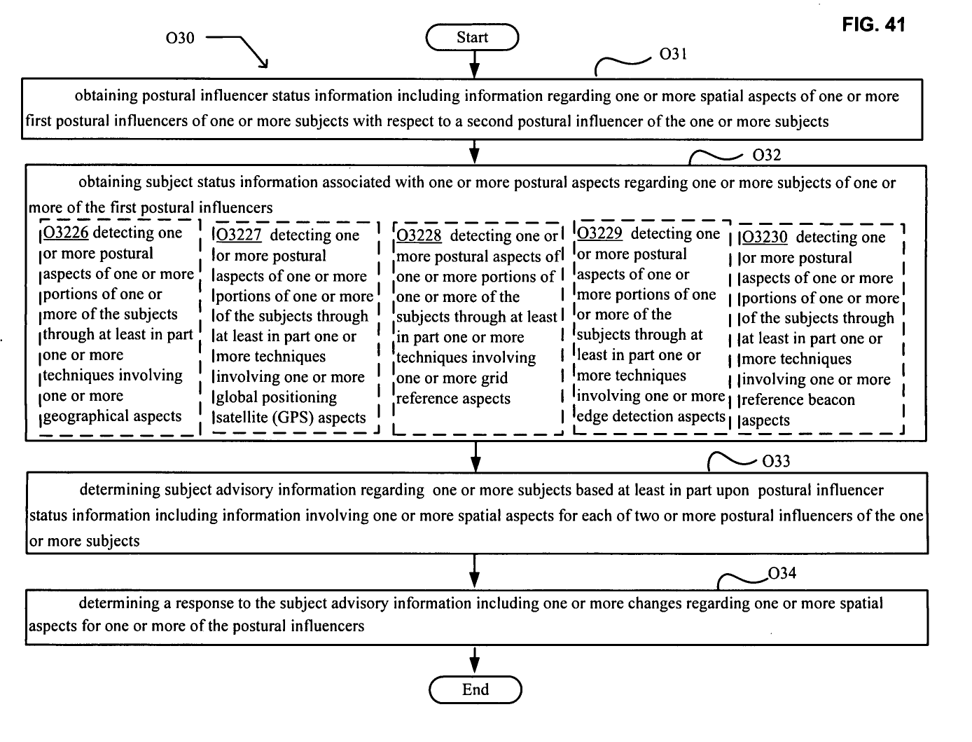

FIG. 41

FIG. 41 illustrates various implementations of the exemplary operation O32 of FIG. 35. In particular, FIG. 41 illustrates example implementations where the operation O32 includes one or more additional operations including, for example, operations O3226, O3227, O3228, O3229, and/or O3230, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O32 may include the operation of O3226 for detecting one or more postural aspects of one or more portions of one or more of the subjects through at least in part one or more techniques involving one or more geographical aspects. An exemplary implementation may include the geographical detecting module 170z of FIG. 7 directing one or more of the image recognition based sensing components 110l of the sensing unit 110 of the status determination system 158 of FIG. 6 including to detect one or more postural aspects of one or more portions of one or more of the subjects 10 through at least in part one or more techniques involving one or more geographical aspects. For example, in some implementations, the transmission D1 from object 1 carrying subject status information regarding the subject 10 and the transmission D2 from the subject carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the image recognition based sensing components 110l of the status determination system 158 can be used to detect postural aspects involving geographical aspects, such as position, location, orientation, and/or conformation of one or more of the subjects 10 in relation to a geographical landmark.

For instance, in some implementations, the exemplary operation O32 may include the operation of O3227 for detecting one or more postural aspects of one or more portions of one or more of the subjects through at least in part one or more techniques involving one or more global positioning satellite (GPS) aspects. An exemplary implementation may include the GPS detecting module 170aa of FIG. 7 directing one or more of the global positioning system (GPS) sensors 108g of one or more of the objects 12 shown in FIG. 10 assigned to monitor one or more of the subjects including to detect one or more postural aspects of the one or more portions of the subject. Postural aspects can include orientation, and/or conformation of the one or more subjects 12 involved and can be sent to the status determination system 158 as transmissions D1 and D2 by the objects as shown in FIG. 11.

For instance, in some implementations, the exemplary operation O32 may include the operation of O3228 for detecting one or more postural aspects of one or more portions of one or more of the subjects through at least in part one or more techniques involving one or more grid reference aspects. An exemplary implementation may include the grid reference detecting module 170ab of FIG. 7 directing one or more of the grid reference based sensing components 110o of the sensing unit 110 of the status determination system 158 of FIG. 6 including to detect one or more postural aspects of one or more portions of one or more of the subject 10 through at least in part one or more techniques involving one or more grid reference aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the grid reference based sensing components 110o of the status determination system 158 can be used to detect postural aspects involving grid reference aspects, such as position, location, orientation, and/or conformation of the subjects 10.

For instance, in some implementations, the exemplary operation O32 may include the operation of O3229 for detecting one or more postural aspects of one or more portions of one or more of the subjects through at least in part one or more techniques involving one or more edge detection aspects. An exemplary implementation may include the edge detecting module 170ac of FIG. 7 directing one or more of the edge detection based sensing components 110p of the sensing unit 110 of the status determination system 158 of FIG. 6 including to detect one or more postural aspects of one or more portions of one or more of the subjects 10 through at least in part one or more techniques involving one or more edge detection aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the edge detection based sensing components 110p of the status determination system 158 can be used to detect postural aspects involving edge detection aspects, such as position, location, orientation, and/or conformation of the subjects 10.

For instance, in some implementations, the exemplary operation O32 may include the operation of O3230 for detecting one or more postural aspects of one or more portions of one or more of the subjects through at least in part one or more techniques involving one or more reference beacon aspects. An exemplary implementation may include the beacon detecting module 170ad of FIG. 7 directing one or more of the reference beacon based sensing components 110q of the sensing unit 110 of the status determination system 158 of FIG. 6 including to detect one or more postural aspects of one or more portions of one or more of the subjects 10 through at least in part one or more techniques involving one or more reference beacon aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the reference beacon based sensing components 110q of the status determination system 158 can be used to detect postural aspects involving reference beacon aspects, such as position, location, orientation, and/or conformation of the subjects 10.

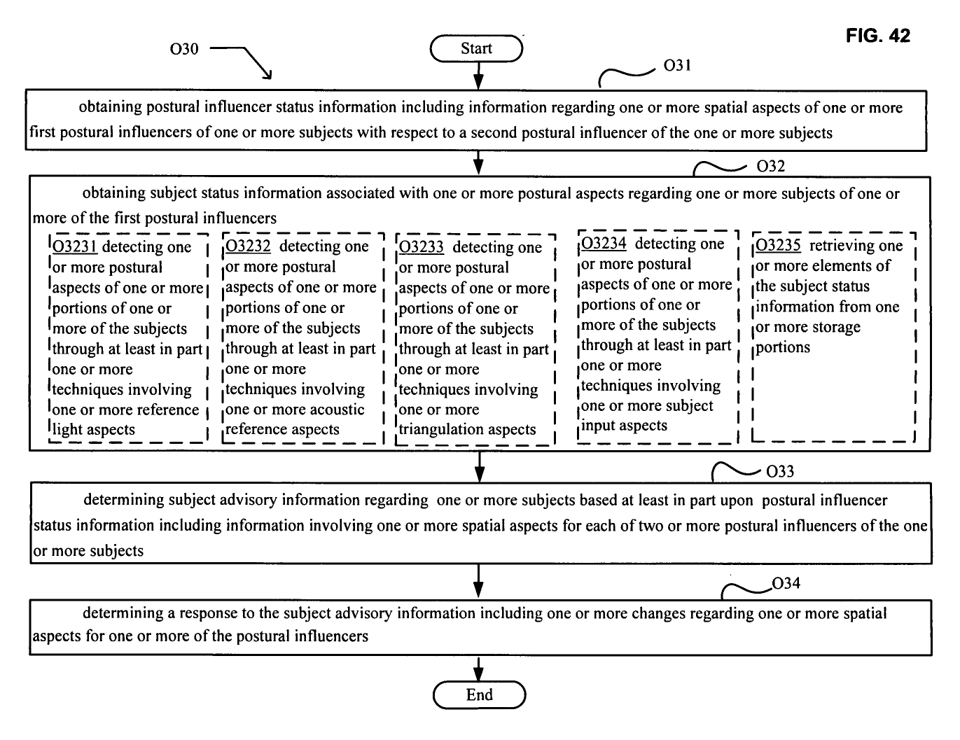

FIG. 42

FIG. 42 illustrates various implementations of the exemplary operation O32 of FIG. 35. In particular, FIG. 42 illustrates example implementations where the operation O32 includes one or more additional operations including, for example, operation O3231, O3232, O3233, O3234, and/or O3235, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O32 may include the operation of O3231 for detecting one or more postural aspects of one or more portions of one or more of the subjects through at least in part one or more techniques involving one or more reference light aspects. An exemplary implementation may include the reference light detecting module 170ae of FIG. 7 directing one or more of the reference light based sensing components 110r of the sensing unit 110 of the status determination system 158 of FIG. 6 including to detect one or more postural aspects of one or more portions of one or more of the subjects 10 through at least in part one or more techniques involving one or more reference light aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the reference light based sensing components 110r of the status determination system 158 can be used to detect postural aspects involving reference light aspects, such as position, location, orientation, and/or conformation of the subjects 10.

For instance, in some implementations, the exemplary operation O32 may include the operation of O3232 for detecting one or more postural aspects of one or more portions of one or more of the subjects through at least in part one or more techniques involving one or more acoustic reference aspects. An exemplary implementation may include the acoustic reference detecting module 170af of FIG. 7 directing one or more of the acoustic reference based sensing components 110s of the sensing unit 110 of the status determination system 158 of FIG. 6 including to detect one or more postural aspects of one or more portions of one or more of the subjects 10 through at least in part one or more techniques involving one or more acoustic reference aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the acoustic reference based sensing components 110s of the status determination system 158 can be used to detect postural aspects involving acoustic reference aspects, such as position, location, orientation, and/or conformation of the subjects 10.

For instance, in some implementations, the exemplary operation O32 may include the operation of O3233 for detecting one or more postural aspects of one or more portions of one or more of the subjects through at least in part one or more techniques involving one or more triangulation aspects. An exemplary implementation may include the triangulation detecting module 170ag of FIG. 7 directing one or more of the triangulation based sensing components 110t of the sensing unit 110 of the status determination system 158 of FIG. 6 including to detect one or more postural aspects of one or more portions of one or more of the subjects 10 through at least in part one or more techniques involving one or more triangulation aspects. For example, in some implementations, the transmission D1 from object 1 carrying subject status information regarding the subject 10 and the transmission D2 from object 2 carrying subject status information about the subject to the status determination system 158, as shown in FIG. 11, will not be present in situations in which the sensors 108 of the object 1 and object 2 are either not present or not being used. Consequently, in cases when the object sensors are not present or are otherwise not used, one or more of the triangulation based sensing components 110t of the status determination system 158 can be used to detect postural aspects involving triangulation aspects, such as position, location, orientation, and/or conformation of the subjects 12.

For instance, in some implementations, the exemplary operation O32 may include the operation of O3234 for detecting one or more postural aspects of one or more portions of one or more of the subjects through at least in part one or more techniques involving one or more subject input aspects. An exemplary implementation may include the subject input module 170ah of FIG. 7 directing subject input aspects as detected by one or more of the contact sensors 108l of the object 12 shown in FIG. 10 assigned to monitor one or more of the subjects 10 including to sense contact such as contact made with the object or another object by the subject 10, such as the subject touching a keyboard device as shown in FIG. 2 to detect one or more postural aspects of one or more portions of the subject. For instance, by sensing contact by the subject 10 (subject) as subject input of one or the objects 12 (device), aspects of the orientation of the subject with respect to the object may be detected.

For instance, in some implementations, the exemplary operation O32 may include the operation of O3235 for retrieving one or more elements of the subject status information from one or more storage portions. An exemplary implementation may include the storage retrieving module 170aj of FIG. 8 directing the control unit 160 of the status determination unit 106 of the status determination system 158 of FIG. 6 including to retrieve one or more elements of subject status information, such as dimensional aspects of one or more of the subjects 10, from one or more storage portions, such as the storage unit 168, as part of obtaining subject status information regarding one or more of the subjects 10.

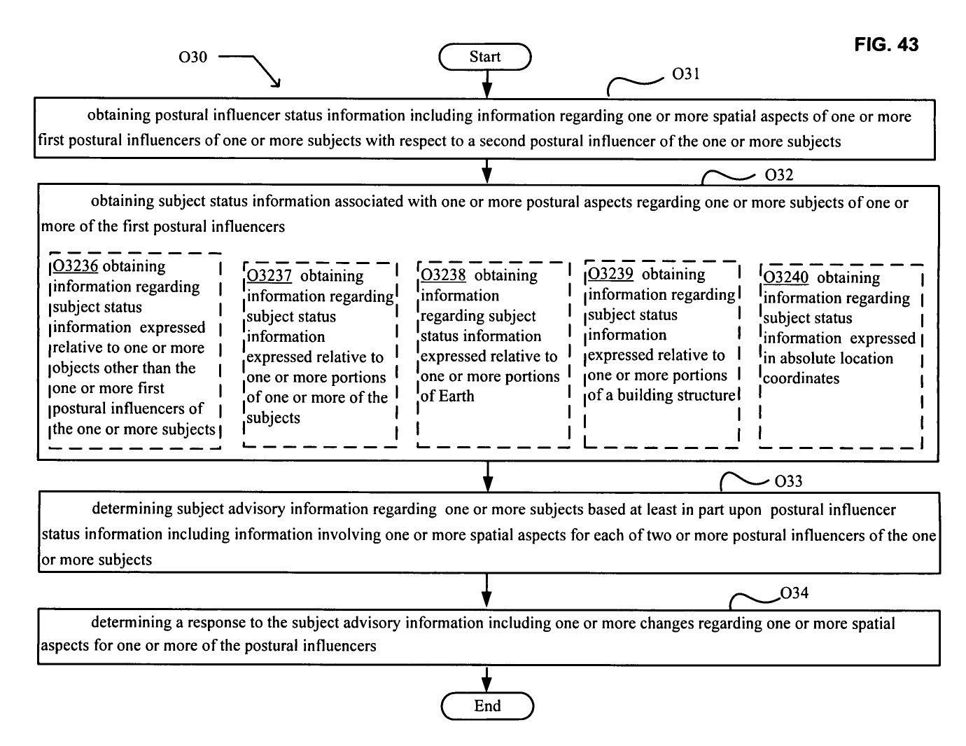

FIG. 43

FIG. 43 illustrates various implementations of the exemplary operation O32 of FIG. 35. In particular, FIG. 43 illustrates example implementations where the operation O32 includes one or more additional operations including, for example, operation O3236, O3237, O3238, O3239, and/or O3240, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O32 may include the operation of O3236 for obtaining information regarding subject status information expressed relative to one or more objects other than the one or more first postural influencers of the one or more subjects. An exemplary implementation may include the object relative obtaining module 170ak of FIG. 8 directing one or more of the sensors 108 of one or more of the objects 12 of FIG. 10 assigned to monitor one or more of the subjects 10 and/or one or more components of the sensing unit 110 of the status determination unit 158 including to obtain information regarding subject status information expressed relative to one or more objects other than the one or more first postural influencers of one or more of the subjects 10. For instance, in some implementations the obtained information can be related to positional or other postural aspects of the subjects 10 as related to one or more of the other objects 14 (such as structural members of a building, artwork, furniture, or other objects) that are not being a first postural influencer of the subject 10 or are otherwise not involved with influencing the subject regarding postural status of the subject. For instance, the postural information obtained can be expressed in terms of distances between one or more of the subjects 10 and the other objects 14.

For instance, in some implementations, the exemplary operation O32 may include the operation of O3237 for obtaining information regarding subject status information expressed relative to one or more portions of one or more of the subjects. An exemplary implementation may include the subject relative obtaining module 170al of FIG. 8 directing one or more of the sensors 108 of the one or more of the objects 12 of FIG. 10 assigned to monitor one or more of the subjects 10 and/or one or more components of the sensing unit 110 of the status determination unit 158 including to obtain information regarding subject status information expressed relative to one or more of the subjects 10. For instance, in some implementations the obtained information can be related to positional or other postural aspects of the subjects 10 and can be expressed such as in terms of distances between the subjects.

For instance, in some implementations, the exemplary operation O32 may include the operation of O3238 for obtaining information regarding subject status information expressed relative to one or more portions of Earth. An exemplary implementation may include the earth relative obtaining module 170am of FIG. 8 directing one or more of the sensors 108 of one or more of the objects 12 of FIG. 10 assigned to monitor one or more of the subjects 10 and/or one or more components of the sensing unit 110 of the status determination unit 158 including to obtain information regarding subject status information expressed relative to one or more portions of the Earth. For instance, in some implementations the obtained information can be expressed relative to global positioning system (GPS) coordinates, geographical features or other aspects, or otherwise expressed relative to one or more portions of Earth.

For instance, in some implementations, the exemplary operation O32 may include the operation of O3239 for obtaining information regarding subject status information expressed relative to one or more portions of a building structure. An exemplary implementation may include the building relative obtaining module 170an of FIG. 8 directing one or more of the sensors 108 of one or more of the objects 12 of FIG. 10 assigned to monitor one or more of the subjects 10 and/or one or more components of the sensing unit 110 of the status determination unit 158 including to obtain information regarding subject status information expressed relative to one or more portions of a building structure. For instance, in some implementations the obtained information can be expressed relative to one or more portions of a building structure that houses the subject 10 and the objects 12 or is nearby to the subject and the objects.

For instance, in some implementations, the exemplary operation O32 may include the operation of O3240 for obtaining information regarding subject status information expressed in absolute location coordinates. An exemplary implementation may include the locational obtaining module 170ao of FIG. 8 directing one or more of the sensors 108 of one or more of the objects 12 of FIG. 10 assigned to monitor one or more of the subjects 10 and/or one or more components of the sensing unit 110 of the status determination unit 158 including to obtain information regarding subject status information expressed in absolute location coordinates. For instance, in some implementations the obtained information can be expressed in terms of global positioning system (GPS) coordinates.

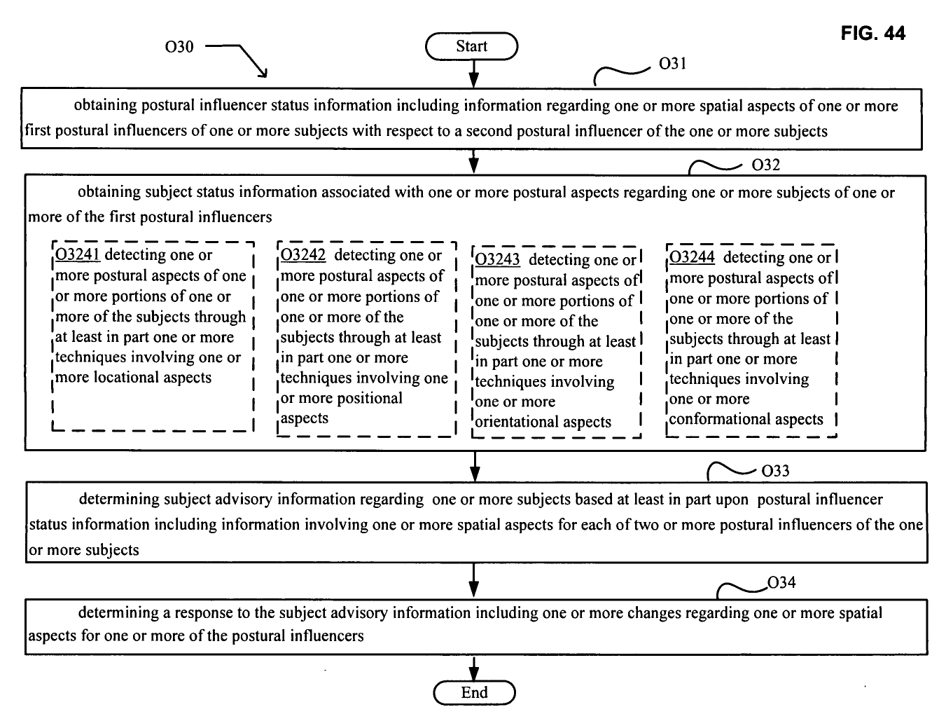

FIG. 44

FIG. 44 illustrates various implementations of the exemplary operation O32 of FIG. 35. In particular, FIG. 44 illustrates example implementations where the operation O32 includes one or more additional operations including, for example, operation O3241, O3242, O3243, and/or O3244, which may be executed generally by, in some instances, one or more of the sensors 108 of the object 12 of FIG. 10 or one or more sensing components of the sensing unit 110 of the status determination system 158 of FIG. 6.

For instance, in some implementations, the exemplary operation O32 may include the operation of O3241 for detecting one or more postural aspects of one or more portions of one or more of the subjects through at least in part one or more techniques involving one or more locational aspects. An exemplary implementation may include the locational detecting module 170ap of FIG. 8 directing one or more of the sensors 108 of one or more of the objects 12 of FIG. 10 assigned to monitor one or more of the subjects 10 and/or one or more components of the sensing unit 110 of the status determination unit 158 including to detect one or more postural aspects of one or more portions of one or more of the subjects 10 through at least in part one or more techniques involving one or more locational aspects. For instance, in some implementations the obtained information can be expressed in terms of global positioning system (GPS) coordinates or geographical coordinates.

For instance, in some implementations, the exemplary operation O32 may include the operation of O3242 for detecting one or more postural aspects of one or more portions of one or more of the subjects through at least in part one or more techniques involving one or more positional aspects. An exemplary implementation may include the positional detecting module 170aq of FIG. 8 directing one or more of the sensors 108 of one or more of the objects 12 of FIG. 10 assigned to monitor one or more of the subjects 10 and/or one or more components of the sensing unit 110 of the status determination unit 158 including to detect one or more postural aspects of one or more portions of one or more of the subjects 10 through at least in part one or more techniques involving one or more positional aspects. For instance, in some implementations the obtained information can be expressed in terms of global positioning system (GPS) coordinates or geographical coordinates.

For instance, in some implementations, the exemplary operation O32 may include the operation of O3243 for detecting one or more postural aspects of one or more portions of one or more of the subjects through at least in part one or more techniques involving one or more orientational aspects. An exemplary implementation may include the orientational detecting module 170ar of FIG. 8 directing one or more of the gyroscopic sensors 108f of one or more of the objects 12 of FIG. 10 assigned to monitor one or more of the subjects 10 as a device shown in FIG. 10 including to detect one or more postural aspects of the one or more portions of the one or more subjects. Postural aspects can include orientation of the subjects 10 involved and can be sent to the status determination system 158 as transmissions D1 and D2 by the objects as shown in FIG. 11.

For instance, in some implementations, the exemplary operation O32 may include the operation of O3244 for detecting one or more postural aspects of one or more portions of one or more of the subjects through at least in part one or more techniques involving one or more conformational aspects. An exemplary implementation may include the conformational detecting module 170 as of FIG. 8 directing the one or more of the gyroscopic sensors 108f of one or more of the objects 12 of FIG. 10 assigned to monitor one or more of the subjects 10 shown in FIG. 10 including to detect one or more postural aspects of the one or more portions of one or more of the subjects. Postural aspects can include conformation of the subjects 10 involved and can be sent to the status determination system 158 as transmissions D1 and D2 by the objects as shown in FIG. 11.

A partial view of a system S100 is shown in FIG. 44 that includes a computer program S104 for executing a computer process on a computing postural influencer. An implementation of the system S100 is provided using a signal-bearing medium S102 bearing one or more instructions determining subject advisory information regarding one or more subjects based at least in part upon postural influencer status information including information involving one or more spatial aspects for each of two or more postural influencers of the one or more subjects may be executed by, for example, the advisory resource unit 102 of the advisory system 118 of FIG. 3. An exemplary implementation may have multiple instances including a first instance in which a first number of objects including at least a portion of the objects 12 depicted in FIG. 2 are arranged in a first configuration and a second instance in which a second number of objects including at least a portion of the objects 1′2 depicted in FIG. 2 are arranged in a second configuration. In the first configuration, not all of the objects depicted in FIG. 2 may be present, for instance the cell device and the RF device may be absent whereas other objects may be present in additional to those depicted in FIG. 2. In the first configuration, the subject 10 can be present or another one or more subjects can be present with first spatial orientations.

The number and configuration of the objects 12 and of the subjects 10 in the second instance can be different than depicted in FIG. 2 and different than the first configuration of the first instance so that the spatial orientations of the objects 12 and the one or more subjects 10 in the second instance can be different than that depicted in FIG. 2 and different than the first instance. The first instance, the second instance, and possible other instances of the multiple instances generally occur at different times to allow for the first configuration, second configuration, and other possible configurations of the objects 12 and/or the one or more subjects 10.

An exemplary implementation may include for a first second instance and a second instance, the advisory resource unit 102 receiving the postural influencer status information associated with the first instance and postural influencer status information associated with the second instance from the status determination unit 106. As depicted in various Figures, the advisory resource unit 102 can be located in various entities including in a standalone version of the advisory system 118 (e.g. see FIG. 3) or in a version of the advisory system included in the object 12 (e.g. see FIG. 13) and the status determination unit can be located in various entities including the status determination system 158 (e.g. see FIG. 11) or in the objects 12 (e.g. see FIG. 14) so that some implementations include the status determination unit sending the postural influencer status information from the communication unit 112 of the status determination system 158 to the communication unit 112 of the advisory system and other implementations include the status determination unit sending the postural influencer status information to the advisory system internally within each of the objects. Once the postural influencer status information is received, the control unit 122 and the storage unit 130 (including in some implementations the guidelines 132) of the advisory resource unit 102 can determine subject advisory information for the first instance and for the second instance, respectively. In some implementations, the subject advisory information is determined by the control unit 122 looking up various portions of the guidelines 132 contained in the storage unit 130 based upon the postural influencer status information. For instance, the postural influencer status information may include locational or positional information for the objects 12 such as those objects depicted in FIG. 2. As an example, the control unit 122 may look up in the storage unit 130 portions of the guidelines associated with this information depicted in FIG. 2 to determine subject advisory information that would inform the subject 10 of FIG. 2 that the subject has been in a posture that over time could compromise integrity of a portion of the subject, such as the trapezius muscle or one or more vertebrae of the subject's spinal column. The subject advisory information could further include one or more suggestions regarding modifications to the existing posture of the subject 10 that may be implemented by repositioning one or more of the objects 12 so that the subject 10 can still use or otherwise interact with the objects in a more desired posture thereby alleviating potential ill effects by substituting the present posture of the subject with a more desired posture. In other implementations, the control unit 122 of the advisory resource unit 102 can include generation of subject advisory information through input of the subject status information into a physiological-based simulation model contained in the memory unit 128 of the control unit, which may then advise of suggested changes to the subject status, such as changes in posture. For each of the first instance and the second instance, the control unit 122 of the advisory resource unit 102 may then determine suggested modifications to the physical status of the objects 12 (devices) based upon the postural influencer status information for the objects that was received. These suggested modifications can be incorporated into the determined subject advisory information for the first instance and determined subject advisory information for the second instance, respectively.

The implementation of the system S100 is also provided using a signal-bearing medium S102 bearing one or more instructions for determining a response to the subject advisory information including one or more changes regarding one or more spatial aspects for one or more of the postural influencers. An exemplary implementation may be executed by, for example, the advisory resource unit 102 of the advisory system 118 of FIG. 3. As an example, for the first instance, once postural influencer status information for the first instance is received, the control unit 122 and the storage unit 130 (including in some implementations the guidelines 132) of the advisory resource unit 102 can determine subject advisory information for the first instance. Based upon the subject advisory information for the first instance, the control 122 of the advisory resource unit 102 of FIG. 3 can generate one or more directions to be stored in the storage 130. For instance, the subject advisory information for the first instance may include an advisory that one or more of the objects 12 be repositioned relative to one or more subjects 10 of the first instance. Directions resulting from generation of the subject advisory information related to the first instance can then include placement and orientation of the objects 12 and one or more of the subjects 10 should all or a portion f them be involved with a future instance. Directions based upon the first instance can be combined and/or modified by the control 122 with directions already and/or to be stored in the storage 130. For instance, directions previously stored in the storage 130 may indicate that a certain health hazard exists such as one or more of the subjects 10 developing a shoulder injury if a portion of a configuration of the objects 12 has a certain characteristic such as requiring one or more of the subjects to assume negative ergonometric postures when interacting with a portion of the objects.

As an example, for the second instance, once postural influencer status information for the second instance is received, the control unit 122 and the storage unit 130 (including in some implementations the guidelines 132) of the advisory resource unit 102 can determine subject advisory information for the second instance. Based upon the subject advisory information for the second instance, the control 122 of the advisory resource unit 102 of FIG. 3 can generate one or more directions to be stored in the storage 130. For instance, the subject advisory information for the second instance may be for one or more of the objects 12 to be repositioned relative to one or more subjects 10 of the second instance. Directions resulting from generation of the subject advisory information related to the second instance can then include placement and orientation of the objects 12 and one or more subjects 10 should all or a portion f them be involved with a future instance. Directions related to the second instance can be modified and/or combined with prior stored directions, such as all or a portion of the directions related to the first instance.

The one or more instructions may be, for example, computer executable and/or logic-implemented instructions. In some implementations, the signal-bearing medium S102 may include a computer-readable medium S106. In some implementations, the signal-bearing medium S102 may include a recordable medium S108. In some implementations, the signal-bearing medium S102 may include a communication medium S110.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those of ordinary skill in the art will recognize that it is common within the art to describe devices and/or processes in the fashion set forth herein, and thereafter use engineering practices to integrate such described devices and/or processes into information processing systems. That is, at least a portion of the devices and/or processes described herein can be integrated into an information processing system via a reasonable amount of experimentation. Those having skill in the art will recognize that a typical information processing system generally includes one or more of a system unit housing, a video display device, a memory such as volatile and non-volatile memory, processors such as microprocessors and digital signal processors, computational entities such as operating systems, drivers, graphical subject interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical information processing system may be implemented utilizing any suitable commercially available components, such as those typically found in information computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably coupleable”, to each other to achieve the desired functionality. Specific examples of operably coupleable include but are not limited to physically mateable and/or physically interacting components and/or wirelessly interactable and/or wirelessly interacting components and/or logically interacting and/or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

All of the above U.S. patents, U.S. patent application publications, U.S. patent applications, foreign patents, foreign patent applications and non-patent publications referred to in this specification and/or listed in any Application Information Sheet are incorporated herein by reference, to the extent not inconsistent herewith.

What is claimed is:

1.-133. (canceled)

134. A system comprising:
circuitry for determining subject advisory information regarding a subject based at least in part upon postural influencer status information including spatial aspect information for two or more postural influencers of the subject; and
circuitry for determining a response to the subject advisory information including directions to change a spatial aspect of at least one of the two or more postural influencers of the subject.

135. The system of claim 134, wherein the circuitry for determining subject advisory information regarding a subject based at least in part upon postural influencer status information including spatial aspect information for two or more postural influencers of the subject comprises:
circuitry for determining subject advisory information including one or more suggested postural influencer locations to locate at least one of the two or more postural influencers.

136. The system of claim 134, wherein the circuitry for determining subject advisory information regarding a subject based at least in part upon postural influencer status information including spatial aspect information for two or more postural influencers of the subject comprises:
circuitry for determining subject advisory information including one or more suggested postural influencer orientations to orient one or more of the postural influencers.

137. The system of claim 134, wherein the circuitry for determining subject advisory information regarding a subject based at least in part upon postural influencer status information including spatial aspect information for two or more postural influencers of the subject comprises:
circuitry for determining subject advisory information including one or more suggested postural influencer positions to position one or more of the postural influencers.

138. The system of claim 134, wherein the circuitry for determining subject advisory information regarding a subject based at least in part upon postural influencer status information including spatial aspect information for two or more postural influencers of the subject comprises:
circuitry for determining subject advisory information including one or more suggested subject conformations to conform one or more of the subjects.

139. The system of claim 134, wherein the circuitry for determining subject advisory information regarding a subject based at least in part upon postural influencer status information including spatial aspect information for two or more postural influencers of the subject comprises:
circuitry for determining subject advisory information including one or more suggested schedules of operation for the subject.

140. The system of claim 134, wherein the circuitry for determining subject advisory information regarding a subject based at least in part upon postural influencer status information including spatial aspect information for two or more postural influencers of the subject comprises:
circuitry for determining subject advisory information including one or more elements of suggested postural adjustment instruction for the subject.

141. The system of claim 134, wherein the two or more postural influencers includes a robotic system and wherein the circuitry for determining subject advisory information regarding a subject based at least in part upon postural influencer status information including spatial aspect information for two or more postural influencers of the subject comprises:
circuitry for determining subject advisory information including spatial aspect information regarding the robotic system.

142. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more radar aspects.

143. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more image capture aspects.

144. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more photographic aspects.

145. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more contact sensing aspects.

146. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more inclinometry aspects.

147. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more pressure aspects.

148. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more geographical aspects.

149. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more global positioning satellite (GPS) aspects.

150. The system of claim 134, wherein the circuitry for determining a response to the subject advisory information including directions to change a spatial aspect of at least one of the two or more postural influencers of the subject comprises:
circuitry for detecting a spatial aspect of at least one of the two or more postural influencers through at least in part one or more techniques involving one or more subject input aspects.

151. The system of claim 183, wherein the circuitry for obtaining the postural influencer status information including the spatial aspect information for the two or more postural influencers of the subject comprises:
circuitry for wirelessly receiving one or more elements of the postural influencer status information from at least one of the two or more postural influencers.

152. The system of claim 183, wherein the circuitry for obtaining the postural influencer status information including the spatial aspect information for the two or more postural influencers of the subject comprises:
circuitry for receiving one or more elements of the postural influencer status information from at least one of the two or more postural influencers via optical communication.

153. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers.

154. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more photographic aspects.

155. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more contact sensing aspects.

156. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more inclinometry aspects.

157. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more geographical aspects.

158. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more edge detection aspects.

159. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more subject input aspects.

160. The system of claim 183, wherein the circuitry for obtaining the postural influencer status information including the spatial aspect information for the two or more postural influencers of the subject comprises:
circuitry for obtaining information regarding postural influencer status information expressed relative to one or more objects other than the first postural influencers of the subject.

161. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more locational aspects.

162. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more conformational aspects.

163. The system of claim 183, wherein the circuitry for obtaining the postural influencer status information including the spatial aspect information for the two or more postural influencers of the subject comprises:
circuitry for detecting one or more spatial aspects of one or more portions of at least one of the two or more postural influencers through at least in part one or more techniques involving one or more visual appearance aspects.

164. The system of claim 134, further comprising:
circuitry for obtaining subject status information associated with one or more postural aspects regarding the subject.

165. The system of claim 164, wherein the circuitry for obtaining subject status information associated with one or more postural aspects regarding the subject comprises:
circuitry for receiving one or more elements of the subject status information via a cellular system.

166. The system of claim 164, wherein the circuitry for obtaining subject status information associated with one or more postural aspects regarding the subject comprises:
circuitry for receiving one or more elements of the subject status information via optical communication.

167. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more radar aspects.

168. The system of claim 164, wherein the circuitry for obtaining subject status information associated with one or more postural aspects regarding the subject comprises: circuitry for detecting one or more postural aspects of one or more portions of the subject through at least in part one or more techniques involving one or more pattern recognition aspects.

169. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more contact sensing aspects.

170. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more inclinometry aspects.

171. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more force aspects.

172. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more geographical aspects.

173. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more grid reference aspects.

174. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more edge detection aspects.

175. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more acoustic reference aspects.

176. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more triangulation aspects.

177. The system of claim 164, wherein the circuitry for obtaining subject status information associated with one or more postural aspects regarding the subject comprises:
circuitry for retrieving one or more elements of the subject status information from one or more storage portions.

178. The system of claim 164, wherein the circuitry for obtaining subject status information associated with one or more postural aspects regarding the subject comprises:
circuitry for obtaining information regarding subject status information expressed relative to one or more portions of the subject.

179. The system of claim 164, wherein the circuitry for obtaining subject status information associated with one or more postural aspects regarding the subject comprises:
circuitry for obtaining information regarding subject status information expressed in absolute location coordinates.

180. The system of claim 164, wherein the circuitry for obtaining subject status information associated with one or more postural aspects regarding the subject comprises:
circuitry for detecting one or more postural aspects of one or more portions of the subject through at least in part one or more techniques involving one or more positional aspects.

181. A system comprising:
means for determining subject advisory information regarding a subject based at least in part upon postural influencer status information including spatial aspect information for two or more postural influencers of the subject; and
means for determining a response to the subject advisory information including directions to change a spatial aspect for at least one of the two or more postural influencers of the subject.

182. A system comprising:
an article of manufacture bearing:
one or more instructions for determining subject advisory information regarding a subject based at least in part upon postural influencer status information including spatial aspect information for two or more postural influencers of the subject; and
one or more instructions for determining a response to the subject advisory information including directions to change a spatial aspect for at least one of the two or more postural influencers of the subject.

183. The system of claim 134, further comprising:
circuitry for obtaining the postural influencer status information including the spatial aspect information for the two or more postural influencers of the subject.
